SEQUENCE LISTING (100 sequences)

SEQ ID NO: 1 - protein, 485 aa, synthetic construct
MPAMVEKGPEVSGKRRGRNNAAASASAAAASAAASAACASPAATAASGAAASSASAAAASAAAAPNNGQNKSLAAAAPNGNSSSNSWEEGSSGSSSDEEHGGGGMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARKQKREREESEDELEEANGNNPIDIEVDQNKESKKEVPPTETVPQVKKEKHSTQAKNRAKRKPPKGMFLSQEDVEAVSANATAATTVLRQLDMELVSVKRQIQNIKQTNSALKEKLDGGIEPYRLPEVIQKCNARWTTEEQLLAVQAIRKYGRDFQAISDVIGNKSVVQVKNFFVNYRRRFNIDEVLQEWEAEHGKEETNGPSNQKPVKSPDNSIKMPEEEDEAPVLDVRYASAS

SEQ ID NO: 2 - DNA, 1458 bp, synthetic construct
  source 1..1458: mol_type = other DNA
atgccggccatggtggagaagggccccgaggtctcagggaagcggagagggaggaacaacgcggccgcctccgcctccgccgccgccgcctccgccgccgcctcggccgcctgcgcctcgccagccgccactgccgcctcgggcgccgccgcctcctcagcctcggccgccgccgcctcagccgccgccgcccccaataatggccagaataaaagtttggcggcggcggcgcccaatggcaacagcagcagcaactcctgggaggaaggcagctcgggctcgtccagcgacgaggagcacggtggcggtggcatgagggtcggaccccagtaccaggcggtggtgcccgacttcgaccccgccaaactggcaagacgcagtcaagaacgggacaatcttggcatgttggtctggtcacccaatcaaaatctgtcagaagcaaagttggatgaatacattgccattgccaaagaaaagcatgggtacaacatggaacaggctcttgggatgctcttctggcataaacataatatcgaaaagtcattggctgatttgcccaactttacccctttcccagatgagtggactgtggaagataaagtcttatttgagcaagcctttagttttcatgggaaaacttttcatagaatccaacaaatgcttccagataaatctatagcaagtctggtgaaattttactattcttggaagaagacgaggactaaaactagtgtgatggatcgccatgcccggaaacaaaaacgggagcgggaggagagcgaggatgaactggaagaggcaaatggaaacaatcccattgacattgaggttgatcaaaacaaggaaagcaaaaaggaggttccccctactgagacagttcctcaggtcaaaaaagaaaaacatagcacacaagctaaaaatagagcaaaaaggaaacctccaaaaggaatgtttctttctcaagaagatgtggaggctgtttctgccaatgccactgctgctaccacggtgctgagacaactagacatggaattggtttcagtcaaacgacagatccagaatattaaacagacaaacagtgctctcaaagaaaaacttgatggtggaatagaaccatatcgacttccagaggtcattcagaaatgtaatgcacgttggactacagaagagcagcttctcgccgtacaagccatcaggaaatatggccgagattttcaggcaatctcagacgtgattgggaacaaatcagtggtacaagtgaaaaacttttttgtaaattatcgacgccgcttcaacatagatgaagttttacaagaatgggaggcagaacatggtaaagaagagaccaatgggcccagtaaccagaagcctgtgaagtccccagataattccattaagatgcccgaagaggaagacgaggctcctgttctggatgtcagatatgcatctgcctcctga

SEQ ID NO: 3 - protein, 523 aa, synthetic construct
MPSVMEKPSAGSGILSRSRAKTVPNGGQPHSEDDSSEEEHSHDSMIRVGTNYQAVIPECKPESPARYSNKELKGMLVWSPNHCVSDAKLDKYIAMAKEKHGYNIEQALGMLLWHKHDVEKSLADLANFTPFPDEWTVEDKVLFEQAFGFHGKCFQRIQQMLPDKLIPSLVKYYYSWKKTRSRTSVMDRQARRLGGRKDKEDSDELEEGRGGVSEGEPDPADPKREPLPSRPLNARPGPGKKEVQVSQYRHHPLRTRRRPPKGMYLSPEGLTAVSGSPDLANLTLRGLDSQLISLKRQVQSMKQTNSSLRQALEGGIDPLRPPEANTKFNSRWTTDEQLLAVQAIRRYGKDFGAIAEVIGNKTLTQVKTFFVSYRRRFNLEEVLQEWEAEQDGAPGAPVPMEEARRGAPLPAPALEEDDEVQITSVSTSVPRSVPPAPPPPPPPTSLSQPPPLLRPPLPTAPTLLRQPPPLQQGRFLQPRLAPNQPPPPLIRPALAAPRHSARPGPQPPPTLIGTPLEPPAPSL

SEQ ID NO: 4 - DNA, 1572 bp, synthetic construct
  source 1..1572: mol_type = other DNA
atgccctcagtgatggagaagccgagcgcgggctctgggatcctgtcccgtagccgggccaagacggtgcccaacggcggacagccccactcggaggatgacagcagcgaggaggagcactcgcacgacagcatgatccgcgttggaaccaattaccaggccgtaattccggagtgcaagcctgagagccccgcacgctacagcaacaaggagctgaaggggatgctggtgtggtcacccaaccactgtgtgtcagatgccaagcttgacaagtacattgcgatggccaaggagaagcatggctacaacattgagcaggcgctgggcatgcttctgtggcataagcacgatgtggagaagtcgctggccgacctggccaacttcaccccattccctgacgagtggacagtagaggacaaggtgctgtttgaacaggcctttggcttccatggcaaatgcttccagcggatccagcagatgctgcctgacaagttgattcccagcctggtgaaatactactactcttggaagaagacccgcagccgaactagtgtgatggacagacaggcccggcggctggggggccgcaaggacaaagaagacagtgatgagctcgaagagggtcgaggaggcgtgagtgagggagagcccgatcctgcagatcccaagagagagcctctaccctctcggcccctgaatgcacgcccaggccctgggaaaaaggaggtccaggtgtctcagtaccgccaccatcccttgcgaacccggcgtcgcccacccaagggcatgtacctgagccctgaaggcctcacggcagtgtcaggaagcccggaccttgccaacctcacgctccgaggtcttgactctcagctcatctccctcaagcgccaggtacagagcatgaagcagacgaacagcagcctgcgccaagccctggagggcggtattgatccactacgccccccggaggccaacaccaagttcaactcccgctggaccacagatgagcagcttttggctgttcaagccatccgtaggtatggcaaagactttggggctattgcagaggtgattgggaacaagactctgacccaggtgaagactttctttgtgagctaccggcgccgcttcaatctggaggaggtgctgcaggaatgggaggctgagcaggatggggcccctggagccccagtccccatggaggaggctaggagaggggctccattgccagccccagccctagaggaagatgatgaggtccagattacatcggtctccacgtccgtgccccgatcagtgccccctgcgccaccaccccctccacctcccacctcgctgtcccagccacccccgctgctgaggccacctttgcccacggctcccactctgctccgacagccacccccactgcagcagggccgcttcctccagccccggctggcccccaaccagcccccaccgcctctcatccgccccgctctggctgccccccgccacagcgcccgccctggccctcagcccccacccaccctgattggaacccctctggagcccccagcaccctcactctga

SEQ ID NO: 5 - protein, 553 aa, synthetic construct
MPGMMEKGPELLGKNRSANGSAKSPAGGGGSGASSTNGGLHYSEPESGCSSDDEHDVGMRVGAEYQARIPEFDPGATKYTDKDNGGMLVWSPYHSIPDAKLDEYIAIAKEKHGYNVEQALGMLFWHKHNIEKSLADLPNFTPFPDEWTVEDKVLFEQAFSFHGKSFHRIQQMLPDKTIASLVKYYYSWKKTRSRTSLMDRQARKLANRHNQGDSDDDVEETHPMDGNDSDYDPKKEAKKEGNTEQPVQTSKIGLGRREYQSLQHRHHSQRSKCRPPKGMYLTQEDVVAVSCSPNAANTILRQLDMELISLKRQVQNAKQVNSALKQKMEGGIEEFKPPESNQKINARWTTEEQLLAVQGVRKYGKDFQAIADVIGNKTVGQVKNFFVNYRRRFNLEEVLQEWEAEQGTQASNGDASTLGEETKSASNVPSGKSTDEEEEAQTPQAPRTLGPSPPAPSSTPTPTAPIATLNQPPPLLRPTLPAAPALHRQPPPLQQQARFIQPRPTLNQPPPPLIRPANSMPPRLNPRPVLSTVGGQQPPSLIGIQTDSQSSLH

SEQ ID NO: 6 - DNA, 1662 bp, synthetic construct
  source 1..1662: mol_type = other DNA
atgcccggcatgatggagaaagggcccgagttactggggaagaaccgatcggccaacggcagcgccaagagcccggcaggcggcggcggcagcggcgcctcgtccaccaacggcgggctgcactactcagagcccgagagcggctgcagcagcgacgacgagcacgatgttgggatgagagtcggagccgaataccaagctcggatccctgaatttgatccaggtgctacaaagtacacagataaagacaatggagggatgcttgtatggtctccatatcacagtatcccagatgccaaattggatgaatacattgcaattgcaaaggaaaagcatggctacaatgtggaacaggcacttggcatgttgttctggcataaacataacattgagaagtcccttgctgatctccctaatttcactccctttccggatgagtggacagtggaagataaagtcctatttgaacaagcctttagttttcatggaaagagctttcacaggattcagcaaatgcttccagataagacaattgcaagccttgtaaaatattactattcttggaaaaaaactcgctctaggacaagtttgatggatcgccaggctcgtaaactagctaatagacataatcagggtgacagtgatgatgatgtagaagaaacacatccaatggatgggaatgatagtgattatgatcccaaaaaagaagccaaaaaagagggtaatactgaacaacctgtccaaactagcaagattggacttggaagaagagagtatcagagtttacaacatcgccatcattctcagcgttctaagtgccgtccacctaagggcatgtatttaacccaggaagatgtggtagcagtttcctgtagtcccaatgcagccaacaccatcctgaggcaactggacatggagttgatctctctaaaacgtcaggttcagaatgctaagcaagtaaacagtgcacttaaacagaaaatggaaggtggaattgaagaattcaaacctcctgagtcaaatcagaaaattaatgcccgttggaccacagaggagcagcttctagcagtgcaaggtgtccgcaaatatggtaaagattttcaagctattgcagatgtaattggcaacaagactgttggccaagtgaagaacttctttgtaaactacaggcgtcggtttaacttagaggaggtattgcaggagtgggaagcagaacaaggaacccaggcttctaatggtgatgcttctactttaggggaggagacaaaaagtgcttctaatgtgccatcagggaagagcactgatgaagaagaggaggcacagaccccacaggctcctcggacactgggtccatcacctcctgccccatcatccactccaacaccaacagcccctattgccactctgaaccagcctccaccacttcttcgtccaacactgcctgctgccccggctcttcaccggcagcctcctccactccagcagcaggctcggttcatccagccccggccaactttaaatcagcctccaccacctcttattcgccctgctaattccatgccaccccgtctaaacccaagaccggtgttgtccacggttggtggtcaacagccaccatcacttattggaattcagacagattcacagtcctcactgcactaa

SEQ ID NO: 7 - protein, 1273 aa, synthetic construct
MKRRLDDQESPVYAAQQRRIPGSTEAFPHQHRVLAPAPPVYEAVSETMQSATGIQYSVTPSYQVSAMPQSSGSHGPAIAAVHSSHHHPTAVQPHGGQVVQSHAHPAPPVAPVQGQQQFQRLKVEDALSYLDQVKLQFGSQPQVYNDFLDIMKEFKSQSIDTPGVISRVSQLFKGHPDLIMGFNTFLPPGYKIEVQTNDMVNVTTPGQVHQIPTHGIQPQPQPPPQHPSQPSAQSAPAPAQPAPQPPPAKVSKPSQLQAHTPASQQTPPLPPYASPRSPPVQPHTPVTISLGTAPSLQNNQPVEFNHAINYVNKIKNRFQGQPDIYKAFLEILHTYQKEQRNAKEAGGNYTPALTEQEVYAQVARLFKNQEDLLSEFGQFLPDANSSVLLSKTTAEKVDSVRNDHGGTVKKPQLNNKPQRPSQNGCQIRRHPTGTTPPVKKKPKLLNLKDSSMADASKHGGGTESLFFDKVRKALRSAEAYENFLRCLVIFNQEVISRAELVQLVSPFLGKFPELFNWFKNFLGYKESVHLETYPKERATEGIAMEIDYASCKRLGSSYRALPKSYQQPKCTGRTPLCKEVLNDTWVSFPSWSEDSTFVSSKKTQYEEHIYRCEDERFELDVVLETNLATIRVLEAIQKKLSRLSAEEQAKFRLDNTLGGTSEVIHRKALQRIYADKAADIIDGLRKNPSIAVPIVLKRLKMKEEEWREAQRGFNKVWREQNEKYYLKSLDHQGINFKQNDTKVLRSKSLLNEIESIYDERQEQATEENAGVPVGPHLSLAYEDKQILEDAAALIIHHVKRQTGIQKEDKYKIKQIMHHFIPDLLFAQRGDLSDVEEEEEEEMDVDEATGAVKKHNGVGGSPPKSKLLFSNTAAQKLRGMDEVYNLFYVNNNWYIFMRLHQILCLRLLRICSQAERQIEEENREREWEREVLGIKRDKSDSPAIQLRLKEPMDVDVEDYYPAFLDMVRSLLDGNIDSSQYEDSLREMFTIHAYIAFTMDKLIQSIVRQLQHIVSDEICVQVTDLYLAENNNGATGGQLNTQNSRSLLESTYQRKAEQLMSDENCFKLMFIQSQGQVQLTIELLDTEEENSDDPVEAERWSDYVERYMNSDTTSPELREHLAQKPVFLPRNLRRIRKCQRGREQQEKEGKEGNSKKTMENVDSLDKLECRFKLNSYKMVYVIKSEDYMYRRTALLRAHQSHERVSKRLHQRFQAWVDKWTKEHVPREMAAETSKWLMGEGLEGLVPCTTTCDTETLHFVSINKYRVKYGTVFKAP

SEQ ID NO: 8 - DNA, 3822 bp, synthetic construct
  source 1..3822: mol_type = other DNA
atgaagcggcgtttggatgaccaggagtcaccggtgtatgcagcccagcagcgtcggatccctggcagcacagaggcttttcctcaccagcaccgggtgcttgcccctgcccctcctgtgtatgaagcagtgtctgagaccatgcagtcagctacgggaattcagtactctgtaacacccagctaccaggtttcagccatgccacagagctccggcagtcatgggcccgctatagcagcagttcatagcagccatcatcacccaacagcggtgcagccccacggaggccaggtggtccagagtcatgctcatccagccccaccagttgcaccagtgcagggacagcagcaatttcagaggctgaaggtggaggatgcgctatcttatcttgaccaggtgaagctgcagtttggtagtcagcctcaggtctacaatgatttccttgacatcatgaaggaatttaaatctcagagcatcgacaccccaggagtgattagtcgtgtgtcccagctattcaaaggccaccccgatctgataatgggattcaacaccttcttgccccctggctacaaaattgaggtgcaaaccaatgacatggtgaatgtgacaactcctggccaggttcatcagattcccacccatggcatccagccacagcctcaaccaccaccccaacatccttcccagccttcagcccagtcagccccagctcctgcccagccagctcctcagcccccacctgccaaagtcagcaagccctcccaactgcaagcacatactccggccagtcagcagactcccccacttccaccgtatgcatccccacgttctccgccggtccagcctcacacaccagtgacaatctcgttgggaacggccccatccttgcagaacaatcaacctgtggagtttaatcatgccatcaactatgttaataagatcaagaacagatttcagggccaaccagacatctacaaagcattcctggagattttgcacacatatcagaaagagcagagaaatgccaaggaagctggaggaaactacactccagcattgacagagcaggaggtgtatgcccaggttgctcgtctctttaaaaaccaggaagatttgttgtcagagtttggacaattcctaccagatgccaacagctccgtgcttttaagcaaaacaactgctgagaaggttgattctgtgagaaatgatcatggaggcactgtcaagaagccccaactgaacaacaagccgcagaggcccagccagaatggctgccagatccgcagacatcctacaggaaccacccctccagttaagaagaaacccaaactgctcaatctgaaggattcttctatggcagatgccagcaaacatggtggtggaacagaatcgttattttttgataaggtccgaaaggctcttcggagtgcagaagcctacgaaaatttcctacgctgtcttgttatttttaaccaggaggtgatctctcgtgctgagcttgtgcaactagtctctcctttcctggggaaattccctgagttgtttaattggtttaaaaactttctgggctataaggagtctgtacatctggaaacttatccaaaggagcgagccacagagggcattgctatggagatagattatgcttcttgtaaacgattgggctccagctatcgagccttaccaaagagttaccagcagcccaagtgtacaggacggactcctctctgtaaagaggttttaaatgatacctgggtttccttcccttcgtggtctgaggactctacctttgtgagttccaagaagactcaatatgaagaacatatttatcgttgtgaagatgaacgctttgagcttgatgtagttttagagaccaatctggcaacaatccgggttctggaagcaatacagaagaagctttcccgcttgtctgctgaagaacaagccaaatttcgcttggacaacacccttgggggcacatcagaagtcatccatagaaaagcactccagaggatatatgctgataaagcagctgacatcattgatggtctgagaaagaatccctccattgctgttccaattgtccttaaaaggttgaagatgaaagaggaagaatggcgagaagctcagagaggctttaacaaagtatggcgagaacaaaatgagaaatactacttgaagtctctggaccaccaggggatcaactttaaacagaatgacaccaaggtcctgaggtctaagagcttactcaatgagattgagagtatctatgatgagaggcaagagcaggctacggaggagaatgctggtgtacctgttggcccacacctctcacttgcgtatgaagacaaacaaatactggaagatgctgctgctctgattatccaccatgtgaagaggcagacaggcattcagaaggaggacaaatataagataaaacaaatcatgcatcattttattccagatttgctctttgcccaaagaggtgatctctcagatgtggaggaagaggaagaagaagagatggatgtagatgaagccacaggggcagttaagaagcacaatggtgttgggggcagtccccctaagtccaagttactgtttagtaacacagcagctcaaaaattaagaggaatggatgaagtatacaacctcttctatgtcaacaacaactggtatatttttatgcgactgcaccagattctctgcctgaggctgctacggatttgttcccaagccgaacggcaaattgaagaagaaaaccgagagagagaatgggaacgggaagtgctgggcataaagcgagacaagagtgacagccctgccattcagctacgtctcaaagaacctatggatgttgatgtagaagattattacccagctttcctggacatggtgcggagcctgctggatggcaacatagactcatcacagtatgaagattcactgagagagatgttcaccattcatgcctacattgcctttaccatggacaaactgatccagagcattgtcagacagctgcagcatatcgtgagtgatgagatctgtgtgcaggtgactgacctttacctggcagaaaataataatggggccaccggaggccagctgaacacacagaactcaaggagcctcctggagtcaacgtatcagcggaaagctgagcagctaatgtcagatgagaattgctttaagcttatgtttattcagagccaaggccaggtccagctgactattgagcttctggacacagaagaggagaattcggatgaccctgtggaagcagagcgctggtcagactacgtggagcgatacatgaattcagatactacctcgcctgagcttcgtgaacatctagcacagaaaccagtatttctccccaggaatctacggcggatccggaagtgtcaacgtggtcgagagcagcaggaaaaggaagggaaggaaggaaacagcaagaagaccatggagaatgtggatagtctggataagctggagtgtagattcaagctgaattcctacaagatggtgtatgtgatcaaatcagaggactatatgtatcggaggaccgccctgctccgggctcatcagtcccatgagcgtgtaagcaagcgtctacatcagagattccaggcctgggtagataaatggaccaaggagcatgtgccccgtgaaatggcagcagagaccagcaagtggctcatgggtgaggggctggagggcctggtgccctgtaccaccacctgtgatacagagaccctgcattttgtgagcattaacaagtatcgtgtcaaatacggcacagtattcaaagccccttaa

SEQ ID NO: 9 - protein, 1162 aa, synthetic construct
MAHAGGGSGGSGAGGPAGRGLSGARWGRSGSAGHEKLPVHVEDALTYLDQVKIRFGSDPATYNGFLEIMKEFKSQSIDTPGVIRRVSQLFHEHPDLIVGFNAFLPLGYRIDIPKNGKLNIQSPLTSQENSHNHGDGAEDFKQQVPYKEDKPQVPLESDSVEFNNAISYVNKIKTRFLDHPEIYRSFLEILHTYQKEQLNTRGRPFRGMSEEEVFTEVANLFRGQEDLLSEFGQFLPEAKRSLFTGNGPCEMHSVQKNEHDKTPEHSRKRSRPSLLRPVSAPAKKKMKLRGTKDLSIAAVGKYGTLQEFSFFDKVRRVLKSQEVYENFLRCIALFNQELVSGSELLQLVSPFLGKFPELFAQFKSFLGVKELSFAPPMSDRSGDGISREIDYASCKRIGSSYRALPKTYQQPKCSGRTAICKELDHWTLLQGSWTDDYCMSKFKNTCWIPGYSAGVLNDTWVSFPSWSEDSTFVSSKKTPYEEQLHRCEDERFELDVVLETNLATIRVLESVQKKLSRMAPEDQEKFRLDDSLGGTSEVIQRRAIYRIYGDKAPEIIESLKKNPVTAVPVVLKRLKAKEEEWREAQQGFNKIWREQYEKAYLKSLDHQAVNFKQNDTKALRSKSLLNEIESVYDEHQEQHSEGRSAPSSEPHLIFVYEDRQILEDAAALISYYVKRQPAIQKEDQGTIHQLLHQFVPSLFFSQQLDLGASEESADEDRDSPQGQTTDPSERKKPAPGPHSSPPEEKGAFGDAPATEQPPLPPPAPHKPLDDVYSLFFANNNWYFFLRLHQTLCSRLLKIYRQAQKQLLEYRTEKEREKLLCEGRREKGSDPAMELRLKQPSEVELEEYYPAFLDMVRSLLEGSIDPTQYEDTLREMFTIHAYVGFTMDKLVQNIARQLHHLVSDDVCLKVVELYLNEKKRGAAGGNLSSRCVRAARETSYQWKAERCMADENCFKVMFLQRKGQVIMTIELLDTEEAQTEDPVEVQHLARYVEQYVGTEGASSSPTEGFLLKPVFLQRNLKKFRRRWQSEQARALRGEARSSWKRLVGVESACDVDCRFKLSTHKMVFIVNSEDYMYRRGTLCRAKQVQPLVLLRHHQHFEEWHSRWLEDNVTVEAASLVQDWLMGEEDEDMVPCKTLCETVHVHGLPVTRYRVQYSRRPASP

SEQ ID NO: 10 - DNA, 3489 bp, synthetic construct
  source 1..3489: mol_type = other DNA
atggcgcacgctggcggtggcagcggtggcagcggtgccggcggccccgcgggccgggggctgagcggcgcccgctggggtcgctcgggctccgcaggccacgagaagctgccggtgcacgtagaagacgccctcacctatctggaccaggtgaagatccgctttggcagcgaccctgccacctacaacggcttcctggagatcatgaaggagttcaaaagccagagcatcgatactcctggagtcatcagacgtgtctcgcagctcttccacgagcaccctgacctcattgttggattcaacgcttttcttcccctcggatatagaatagacattcccaagaatggcaagttaaacatacagtcgcctctgacaagccaggagaattcgcacaaccacggggacggtgcagaggacttcaagcagcaggtgccgtataaagaggacaaaccccaggtgcccctggagtccgattccgtggaattcaacaacgccatcagctatgtgaataagattaaaacccgcttcctagaccacccagaaatctacaggtcattcctggagatcctgcacacgtaccagaaggagcagctgaacacgaggggccggccattccgaggcatgtctgaagaggaggtgttcaccgaggtggccaacctcttccggggccaggaggacctgctctcagagtttggacagttcctgcccgaagccaagcggtctctgttcacaggaaacgggccgtgcgagatgcacagcgtgcagaagaacgagcacgacaagaccccggagcacagcaggaagcgctcccggccctcgctcctccgccccgtgtctgcacccgccaagaaaaaaatgaaacttcgtggtaccaaagacctgtccatcgctgcagtggggaagtacgggactctgcaggaattttctttctttgacaaggtccgccgggtgctgaagagccaggaggtgtatgaaaacttcctccgctgcatcgcactcttcaaccaggagctggtgtctggctctgagctcctgcagctcgtcagcccatttctggggaaatttccagaactctttgcacagttcaagtccttcctgggggtaaaagagctgtccttcgcgccacccatgagcgacagatccggggacgggataagccgggaaattgattatgcatcctgcaagcgcataggatccagctaccgggcactccccaaaacctaccagcagcccaagtgcagtgggaggacagccatctgcaaggagcttgaccattggacacttctccagggttcgtggacagacgattactgcatgtccaagttcaagaatacctgctggattccaggatatagtgcaggggtactgaacgacacctgggtctccttcccttcctggtctgaggactccacgttcgtcagctccaagaagacaccgtacgaggagcagcttcaccgctgtgaggacgagcgcttcgagttagacgttgtcctggagacgaacctggccacaatccgtgtgttggaaagtgtgcagaagaagctgtctcggatggcgccggaagaccaggagaagttccggctggacgactccctgggaggcacgtcggaggtgatccagcgccgtgccatttatcgcatctatggcgacaaggccccggagatcatcgagagcctcaagaagaaccctgtcaccgctgtccccgttgtcctgaaaagactgaaggccaaggaagaggagtggcgggaggcccagcagggcttcaacaagatctggcgggagcagtatgagaaggcgtacctcaagtcccttgaccaccaggctgtgaacttcaagcagaacgacaccaaggccctgcgctccaagagcttgctcaacgagatcgagagcgtctacgacgagcaccaggagcagcactcggagggccgcagtgccccctctagcgagccgcacctcatctttgtgtacgaggaccggcagatcctggaggacgcagcagcgctcatcagctactacgtgaagcggcagccggccatccagaaggaggaccagggcaccatccaccagctgctgcaccagttcgtgcccagcctcttcttctctcagcagctggacctgggcgcctccgaggagtcagctgatgaggaccgggacagcccccaggggcagaccacagaccccagtgagcggaagaagccggcgccaggaccccacagtagccccccagaggagaagggggccttcggggatgccccggccactgagcagccacccctgccgcccccagccccgcacaagcccctggacgatgtctacagcctattttttgccaacaacaactggtacttcttcctgcgcctgcaccagaccctgtgctccaggctgctgaagatctaccgccaggcgcagaagcagcttctggagtatcggaccgagaaggagcgggagaagctgctgtgtgagggccgcagggagaagggcagcgaccccgccatggagctgcggctgaagcagcccagtgaagtggagctggaggagtactacccggccttcctggacatggtgcggagcctgctggagggcagcatcgaccccacgcagtacgaggacaccctacgcgagatgttcaccatccatgcctacgtgggcttcaccatggacaagctggtgcagaacattgcgcggcagctgcaccacctcgtgagcgatgacgtctgcctgaaggtggtggagctctacctgaacgagaagaagcggggtgccgctggtgggaacctgtcctcccgctgcgtccgcgctgctagggagaccagctaccagtggaaggctgagcgctgcatggccgacgagaactgcttcaaggtgatgttcctgcagcgcaaagggcaggtgatcatgaccatcgagctcctggacaccgaggaggcccagacggaggaccctgtggaggtccagcacctggctcggtacgtggagcagtatgtggggaccgagggcgcgtccagctcgcccactgagggcttcctcctgaaacctgtgttcctgcagaggaacctcaagaagttccgccgccggtggcagagcgagcaggcgcgggccctgcgcggtgaggccaggagctcctggaagcggctggtgggcgtggagagcgcctgcgacgtggactgccgcttcaagctcagcactcacaagatggtgttcatcgtgaactccgaggactacatgtaccgtcgcgggaccctctgccgggccaagcaggtgcagcccctggtcctgctccgccaccaccagcactttgaggagtggcacagccgctggctggaggacaatgtgacggtggaggcggctagcctggtgcaggactggctgatgggtgaggaggacgaggacatggtaccctgcaagacgctgtgtgagacagtgcacgtgcacggcctgcccgtgacccgctaccgcgtgcagtacagccgccgcccggcctcgccctga

SEQ ID NO: 11 - protein, 482 aa, synthetic construct
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 12 - DNA, 1449 bp, synthetic construct
  source 1..1449: mol_type = other DNA
atggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctga

SEQ ID NO: 13 - protein, 488 aa, synthetic construct
MAYSQGGGKKKVCYYYDGDIGNYYYGQGHPMKPHRIRMTHNLLLNYGLYRKMEIYRPHKATAEEMTKYHSDEYIKFLRSIRPDNMSEYSKQMQRFNVGEDCPVFDGLFEFCQLSTGGSVAGAVKLNRQQTDMAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNFPMRDGIDDESYGQIFKPIISKVMEMYQPSAVVLQCGADSLSGDRLGCFNLTVKGHAKCVEVVKTFNLPLLMLGGGGYTIRNVARCWTYETAVALDCEIPNELPYNDYFEYFGPDFKLHISPSNMTNQNTPEYMEKIKQRLFENLRMLPHAPGVQMQAIPEDAVHEDSGDEDGEDPDKRISIRASDKRIACDEEFSDSEDEGEGGRRNVADHKKGAKKARIEEDKKETEDKKTDVKEEDKSKDNSGEKTDTKGTKSEQLSNP

SEQ ID NO: 14 - DNA, 1467 bp, synthetic construct
  source 1..1467: mol_type = other DNA
atggcgtacagtcaaggaggcggcaaaaaaaaagtctgctactactacgacggtgatattggaaattattattatggacagggtcatcccatgaagcctcatagaatccgcatgacccataacttgctgttaaattatggcttatacagaaaaatggaaatatataggccccataaagccactgccgaagaaatgacaaaatatcacagtgatgagtatatcaaatttctacggtcaataagaccagataacatgtctgagtatagtaagcagatgcagagatttaatgttggagaagattgtccagtgtttgatggactctttgagttttgtcagctctcaactggcggttcagttgctggagctgtgaagttaaaccgacaacagactgatatggctgttaattgggctggaggattacatcatgctaagaaatcagaagcatcaggattctgttacgttaatgatattgtgcttgccatccttgaattactaaagtatcatcagagagtcttatatattgatatagatattcatcatggtgatggtgttgaagaagctttttatacaacagatcgtgtaatgacggtatcattccataaatatggggaatactttcctggcacaggagacttgagggatattggtgctggaaaaggcaaatactatgctgtcaattttccaatgagagatggtatagatgatgagtcatatgggcagatatttaagcctattatctcaaaggtgatggagatgtatcaacctagtgctgtggtattacagtgtggtgcagactcattatctggtgatagactgggttgtttcaatctaacagtcaaaggtcatgctaaatgtgtagaagttgtaaaaacttttaacttaccattactgatgcttggaggaggtggctacacaatccgtaatgttgctcgatgttggacatatgagactgcagttgcccttgattgtgagattcccaatgagttgccatataatgattactttgagtattttggaccagacttcaaactgcatattagtccttcaaacatgacaaaccagaacactccagaatatatggaaaagataaaacagcgtttgtttgaaaatttgcgcatgttacctcatgcacctggtgtccagatgcaagctattccagaagatgctgttcatgaagacagtggagatgaagatggagaagatccagacaagagaatttctattcgagcatcagacaagcggatagcttgtgatgaagaattctcagattctgaggatgaaggagaaggaggtcgaagaaatgtggctgatcataagaaaggagcaaagaaagctagaattgaagaagataagaaagaaacagaggacaaaaaaacagacgttaaggaagaagataaatccaaggacaacagtggtgaaaaaacagataccaaaggaaccaaatcagaacagctcagcaacccctga

SEQ ID NO: 15 - protein, 876 aa, synthetic construct
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGQAGGLQDDSSGGYGDGQASGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQADTVKVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 16 - DNA, 2631 bp, synthetic construct
  source 1..2631: mol_type = other DNA
atgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtga

SEQ ID NO: 17 - protein, 680 aa, synthetic construct
MELQTLQEALKVEIQVHQKLVAQMKQDPQNADLKKQLHELQAKITALSEKQKRVVEQLRKNLIVKQEQPDKFQIQPLPQSENKLQTAQQQPLQQLQQQQQYHHHHAQQSAAASPNLTASQKTVTTASMITTKTLPLVLKAATATMPASVVGQRPTIAMVTAINSQKAVLSTDVQNTPVNLQTSSKVTGPGAEAVQIVAKNTVTLVQATPPQPIKVPQFIPPPRLTPRPNFLPQVRPKPVAQNNIPIAPAPPPMLAAPQLIQRPVMLTKFTPTTLPTSQNSIHPVRVVNGQTATIAKTFPMAQLTSIVIATPGTRLAGPQTVQLSKPSLEKQTVKSHTETDEKQTESRTITPPAAPKPKREENPQKLAFMVSLGLVTHDHLEEIQSKRQERKRRTTANPVYSGAVFEPERKKSAVTYLNSTMHPGTRKRGRPPKYNAVLGFGALTPTSPQSSHPDSPENEKTETTFTFPAPVQPVSLPSPTSTDGDIHEDFCSVCRKSGQLLMCDTCSRVYHLDCLDPPLKTIPKGMWICPRCQDQMLKKEEAIPWPGTLAIVHSYIAYKAAKEEEKQKLLKWSSDLKQEREQLEQKVKQLSNSISKCMEMKNTILARQKEMHSSLEKVKQLIRLIHGIDLSKPVDSEATVGAISNGPDCTPPANAATSTPAPSPSSQSCTANCNQGEETK

SEQ ID NO: 18 - DNA, 2043 bp, synthetic construct
  source 1..2043: mol_type = other DNA
atggagttgcagactctacaggaggctcttaaagtggaaattcaggttcaccagaaactggttgctcaaatgaagcaggatccacagaatgctgacttaaagaaacagcttcatgaactccaagccaaaatcacagctttgagtgagaaacagaaaagagtagttgaacagctacggaagaacctgatagtaaagcaagaacaaccggacaagttccaaatacagccattgccacaatctgaaaacaaactacaaacagcacagcagcaaccactacagcaactacaacaacagcagcagtaccaccaccaccacgcccagcagtcagctgcagcctctcccaacctgactgcttcacagaagactgtaactacagcttctatgattaccacaaagacactacctctcgtcttgaaagcagcaactgcgaccatgcctgcctctgtggtgggccagagacctaccattgctatggtgaccgccatcaacagtcagaaggctgtgctcagcactgatgtgcagaacacaccagtcaacctccagacgtctagtaaggtcactgggcctggggcagaggctgtccaaattgtggcaaaaaacacagtcactctggttcaggcaacacctcctcagcccatcaaagtaccacagtttatcccccctcctagactcactccacgtccaaactttcttccacaggttcgacccaagcctgtggcccagaataacattcctattgccccagcaccacctcccatgctcgcagctcctcagcttatccagaggcccgtcatgctgaccaagttcacccccacaacccttcccacatcccagaattccatccaccccgtccgtgtcgtcaatgggcagactgcaaccatagccaaaacgttccccatggcccagctcaccagcattgtgatagctactccagggaccagactcgctggacctcaaactgtacagcttagcaagccaagtcttgaaaaacagacagttaaatctcacacagaaacagatgagaaacaaacagagagccgcaccatcaccccacctgctgcacccaaaccaaaacgggaggagaaccctcagaaacttgccttcatggtgtctctagggttggtaacacatgaccatctagaagaaatccaaagcaagaggcaagagcgaaaaagaagaacaacagcaaatccggtctacagtggagcagtctttgagccagagcgtaagaagagtgcagtgacatacctaaacagcacaatgcaccctgggacccggaagagaggtcgtcctccaaaatacaatgcagtgctggggtttggagcccttaccccaacatccccccaatccagtcatcctgactcccctgaaaatgaaaagacagagaccacattcactttccctgcacctgttcagcctgtgtccctgcccagccccacctccacagacggtgatattcatgaggatttttgcagcgtttgcagaaaaagtggccagttactgatgtgcgacacatgttcccgtgtatatcatttggactgcttagacccccctctgaaaacaattcccaagggcatgtggatctgtcccagatgtcaggaccagatgctgaagaaggaagaagcaattccatggcctggaactttagcaattgttcattcctatattgcctacaaagcagcaaaagaagaagagaaacagaagttacttaaatggagttcagatttaaaacaagaacgagaacaactagagcaaaaggtgaaacagctcagcaattccataagtaaatgcatggaaatgaagaacaccatcctggcccggcagaaggagatgcacagctccctggagaaggtaaaacagctgattcgcctcatccacggcatcgacctctccaaacctgtagactctgaggccactgtgggggccatctccaatggcccggactgcaccccccctgccaatgccgccacctccacgccggccccttccccctcctcccagagctgcacagcgaactgtaaccagggggaagagactaaataa

SEQ ID NO: 19 - protein, 429 aa, synthetic construct
MSGGVYGGDEVGALVFDIGSYTVRAGYAGEDCPKVDFPTAIGMVVERDDGSTLMEIDGDKGKQGGPTYYIDTNALRVPRENMEAISPLKNGMVEDWDSFQAILDHTYKMHVKSEASLHPVLMSEAPWNTRAKREKLTELMFEHYNIPAFFLCKTAVLTAFANGRSTGLILDSGATHTTAIPVHDGYVLQQGIVKSPLAGDFITMQCRELFQEMNIELVPPYMIASKEAVREGSPANWKRKEKLPQVTRSWHNYMCNCVIQDFQASVLQVSDSTYDEQVAAQMPTVHYEFPNGYNCDFGAERLKIPEGLFDPSNVKGLSGNTMLGVSHVVTTSVGMCDIDIRPGLYGSVIVAGGNTLIQSFTDRLNRELSQKTPPSMRLKLIANNTTVERRFSSWIGGSILASLGTFQQMWISKQEYEEGGKQCVERKCP

SEQ ID NO: 20 - DNA, 1290 bp, synthetic construct
  source 1..1290: mol_type = other DNA
atgagcggcggcgtgtacgggggagatgaagttggagcccttgtttttgacattggatcctatactgtgagagctggttatgctggtgaggactgccccaaggtggattttcctacagctattggtatggtggtagaaagagatgacggaagcacattaatggaaatagatggcgataaaggcaaacaaggcggtcccacctactacatagatactaatgctctgcgtgttccgagggagaatatggaggccatttcacctctaaaaaatgggatggttgaagactgggatagtttccaagctattttggatcatacctacaaaatgcatgtcaaatcagaagccagtctccatcctgttctcatgtcagaggcaccgtggaatactagagcaaagagagagaaactgacagagttaatgtttgaacactacaacatccctgccttcttcctttgcaaaactgcagttttgacagcatttgctaatggtcgttctactgggctgattttggacagtggagccactcataccactgcaattccagtccacgatggctatgtccttcaacaaggcattgtgaaatcccctcttgctggagactttattactatgcagtgcagagaactcttccaagaaatgaatattgaattggttcctccatatatgattgcatcaaaagaagctgttcgtgaaggatctccagcaaactggaaaagaaaagagaagttgcctcaggttacgaggtcttggcacaattatatgtgtaattgtgttatccaggattttcaagcttcggtacttcaagtgtcagattcaacttatgatgaacaagtggctgcacagatgccaactgttcattatgaattccccaatggctacaattgtgattttggtgcagagcggctaaagattccagaaggattatttgacccttccaatgtaaaggggttatcaggaaacacaatgttaggagtcagtcatgttgtcaccacaagtgttgggatgtgtgatattgacatcagaccaggtctctatggcagtgtaatagtggcaggaggaaacacactaatacagagttttactgacaggttgaatagagagctgtctcagaaaactcctccaagtatgcggttgaaattgattgcaaataatacaacagtggaacggaggtttagctcatggattggcggctccattctagcctctttgggtacctttcaacagatgtggatttccaagcaagaatatgaagaaggagggaagcagtgtgtagaaagaaaatgcccttga

SEQ ID NO: 21 - protein, 1210 aa, synthetic construct
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 22 - DNA, 3633 bp, synthetic construct
  source 1..3633: mol_type = other DNA
atggcggcggcggcgggagctgcagcggcggcggccgccgagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatga

SEQ ID NO: 23 - protein, 494 aa, synthetic construct
MPLYSVTVKWGKEKFEGVELNTDEPPMVFKAQLFALTGVQPARQKVMVKGGTLKDDDWGNIKIKNGMTLLMMGSADALPEEPSAKTVFVEDMTEEQLASAMELPCGLTNLGNTCYMNATVQCIRSVPELKDALKRYAGALRASGEMASAQYITAALRDLFDSMDKTSSSIPPIILLQFLHMAFPQFAEKGEQGQYLQQDANECWIQMMRVLQQKLEAIEDDSVKETDSSSASAATPSKKKSLIDQFFGVEFETTMKCTESEEEEVTKGKENQLQLSCFINQEVKYLFTGLKLRLQEEITKQSPTLQRNALYIKSSKISRLPAYLTIQMVRFFYKEKESVNAKVLKDVKFPLMLDMYELCTPELQEKMVSFRSKFKDLEDKKVNQQPNTSDKKSSPQKEVKYEPFSFADDIGSNNCGYYDLQAVLTHQGRSSSSGHYVSWVKRKQDEWIKFDDDKVSIVTPEDILRLSGGGDWHIAYVLLYGPRRVEIMEEESEQ

SEQ ID NO: 24 - DNA, 1485 bp, synthetic construct
  source 1..1485: mol_type = other DNA
atgccgctctactccgttactgtaaaatggggaaaggagaaatttgaaggtgtagaattgaatacagatgaacctccaatggtattcaaggctcagctgtttgcgttgactggagtccagcctgccagacagaaagttatggtgaaaggaggaacgctaaaggatgatgattggggaaacatcaaaataaaaaatggaatgactctactaatgatggggtcagcagatgctcttccagaagaaccctcagccaaaactgtcttcgtagaagacatgacagaagaacagttagcatctgctatggagttaccatgtggattgacaaaccttggtaacacttgttacatgaatgccacagttcagtgtattcgttctgtgcctgaactcaaagatgcccttaaaaggtatgcaggtgccttgagagcttcaggggaaatggcttcagcgcagtatattactgcagcccttagagatttgtttgattccatggataaaacttcttccagtattccacctattattctactgcagtttttgcacatggctttcccacagtttgccgagaaaggtgaacaaggacagtatcttcaacaggatgctaatgaatgttggatacaaatgatgcgagtattgcaacagaaattggaagcaatagaggatgattctgttaaagagacagactcctcatctgcatcggcagcgacaccttctaaaaagaaaagtttaatcgatcagttcttcggtgttgagtttgaaactaccatgaaatgtacagaatctgaagaagaagaagtcaccaaaggaaaggaaaatcaacttcagcttagctgttttatcaatcaggaagtcaagtatctttttacaggacttaaattgcgacttcaggaagaaatcaccaaacagtctccaacgttgcaaagaaatgccttgtatatcaaatcttccaagatcagccggctgcctgcttacttgaccattcagatggttcgatttttttataaagagaaggaatctgtgaatgccaaagttcttaaggatgttaaatttcctcttatgttggatatgtatgaactgtgtacaccagaacttcaagagaaaatggtgtcttttcgatccaaattcaaggatctagaagataaaaaagtgaatcagcagccaaatacaagtgacaaaaagagtagtccccagaaagaagttaagtatgaacccttttcttttgctgatgatattggctccaataattgtggatactatgacttacaagcagtactaacacaccagggaaggtctagttcttcaggtcattatgtatcatgggtgaaaaggaaacaagatgaatggattaagtttgatgatgacaaagtcagcatcgtaacaccagaagatatcttacggctttctggtggtggagactggcatatcgcttacgttctactctatgggcctcgcagagttgaaataatggaagaggaaagtgaacagtaa

SEQ ID NO: 25 - protein, 326 aa, synthetic construct
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 26 - DNA, 981 bp, synthetic construct
  source 1..981: mol_type = other DNA
atgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctatggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcacagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataa

SEQ ID NO: 27 - protein, 1679 aa, synthetic construct
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKKITGKDIHDTASSVARGLQFQRGLQFCTRASKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 28 - DNA, 5040 bp, synthetic construct
  source 1..5040: mol_type = other DNA
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaaaattacaggaaaagatatccatgacacagccagcagtgtggcacgtgggctacaattccagcgtggccttcagttctgcacacgtgcgtcaaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactga

SEQ ID NO: 29 - protein, 751 aa, synthetic construct
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 30 - DNA, 2256 bp, synthetic construct
  source 1..2256: mol_type = other DNA
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 31 - protein, 544 aa, synthetic construct
MASEELYEVERIVDKRKNKKGKTEYLVRWKGYDSEDDTWEPEQHLVNCEEYIHDFNRRHTEKQKESTLTRTNRTSPNNARKQISRSTNSNFSKTSPKALVIGKDHESKNSQLFAASQKFRKNTAPSLSSRKNMDLAKSGIKILVPKSPVKSRTAVDGFQSESPEKLDPVEQGQEDTVAPEVAAEKPVGALLGPGAERARMGSRPRIHPLVPQVPGPVTAAMATGLAVNGKGTSPFMDALTANGTTNIQTSVTGVTASKRKFIDDRRDQPFDKRLRFSVRQTESAYRYRDIVVRKQDGFTHILLSTKSSENNSLNPEVMREVQSALSTAAADDSKLVLLSAVGSVFCCGLDFIYFIRRLTDDRKRESTKMAEAIRNFVNTFIQFKKPIIVAVNGPAIGLGASILPLCDVVWANEKAWFQTPYTTFGQSPDGCSTVMFPKIMGGASANEMLLSGRKLTAQEACGKGLVSQVFWPGTFTQEVMVRIKELASCNPVVLEESKALVRCNMKMELEQANERECEVLKKIWGSAQGMDSMLKYLQRKIDEF

SEQ ID NO: 32 - DNA, 1635 bp, synthetic construct
  source 1..1635: mol_type = other DNA
atggcttccgaggagctgtacgaggttgaaaggattgttgacaaaaggaaaaataaaaaagggaagacagagtatttggttcggtggaaaggctatgacagcgaggacgacacttgggagccggaacagcacctcgtgaactgtgaggaatacatccacgacttcaacagacgccacacggagaagcagaaggagagcacattgaccagaacaaacaggacctctcccaacaatgctaggaaacaaatctccagatccaccaacagcaacttttctaagacctctcctaaggcactcgtgattgggaaagaccacgaatccaaaaacagccagctgtttgctgccagccagaagttcaggaagaacacagctccatctctctccagccggaagaacatggacctagcgaagtcaggtatcaagatcctcgtgcctaaaagccccgttaagagcaggaccgcagtggacggctttcagagcgagagccctgagaaactggaccccgtcgagcagggtcaggaggacacagtggcacccgaagtggcagcggaaaagccggtcggagctttattgggccccggtgccgagagggccaggatggggagcaggcccaggatacacccactagtgcctcaggtgcccggccctgtgactgcagccatggccacaggcttagctgttaacgggaaaggtacatctccgttcatggatgcattaacagccaatgggacaaccaacatacagacatctgttacaggagtgactgccagcaaaaggaaatttattgacgacagaagagaccagccttttgacaagcgattgcgtttcagcgtgaggcaaacagaaagtgcctacagatacagagatattgtggtcaggaagcaggatggcttcacccacatcttgttatccacaaagtcctcagagaataactcactaaatccagaggtaatgagagaagtccagagtgctctgagcacggccgctgccgatgacagcaagctggtactgctcagcgccgttggcagcgtcttctgttgtggacttgactttatttattttatacgacgtctgacagatgacaggaaaagagaaagcactaaaatggcagaagctatcagaaacttcgtgaatactttcattcaatttaagaagcccattattgtagcagtcaatggcccagccattggtctaggagcatctatattgcctctttgcgatgtggtttgggctaatgaaaaggcttggtttcaaacaccctataccaccttcggacagagtccagatggctgttctaccgttatgtttcccaagataatgggaggagcatctgcaaacgagatgctgctcagtggacggaagctgacagcgcaggaggcgtgtggcaagggcctggtctcccaggtgttttggcccgggacgttcactcaggaagtgatggttcgcattaaggagcttgcctcgtgcaatccagttgtgcttgaggaatccaaagccctcgtgcgctgcaacatgaagatggagctggagcaggccaacgagagggagtgtgaggtgctgaagaaaatctggggctcggcccaggggatggactccatgttaaagtacttgcagaggaagatcgatgagttctga

SEQ ID NO: 33 - protein, 317 aa, synthetic construct
MSHGPKQPGAAAAPAGGKAPGQHGGFVVTVKQERGEGPRAGEKGSHEEEPVKKRGWPKGKKRKKILPNGPKAPVTGYVRFLNERREQIRTRHPDLPFPEITKMLGAEWSKLQPTEKQRYLDEAEREKQQYMKELRAYQQSEAYKMCTEKIQEKKIKKEDSSSGLMNTLLNGHKGGDCDGFSTFDVPIFTEEFLDQNKAREAELRRLRKMNVAFEEQNAVLQRHTQSMSSARERLEQELALEERRTLALQQQLQAVRQALTASFASLPVPGTGETPTLGTLDFYMARLHGAIERDPAQHEKLIVRIKEILAQVASEHL

SEQ ID NO: 34 - DNA, 954 bp, synthetic construct
  source 1..954: mol_type = other DNA
atgtcccacggccccaagcagcccggcgcggccgccgcgccggcgggcggcaaggctccgggccagcatgggggcttcgtggtgactgtcaagcaagagcgcggcgagggtccacgcgcgggcgagaaggggtcccacgaggaggagccggtgaagaaacgcggctggcccaagggcaagaagcggaagaagattctgccgaatgggcccaaggcaccggtcacgggctacgtgcgcttcctgaacgagcggcgcgagcagatccgcacgcgccacccggatctgccctttcccgagatcaccaagatgctgggcgccgagtggagcaagctgcagccaacggaaaagcagcggtacctggatgaggccgagagagagaagcagcagtacatgaaggagctgcgggcgtaccagcagtctgaagcctataagatgtgcacggagaagatccaggagaagaagatcaagaaagaagactcgagctctgggctcatgaacactctcctgaatggacacaagggtggggactgcgatggcttctccaccttcgatgttcccatcttcactgaagagttcttggaccaaaacaaagcgcgtgaggcggagcttcggcgcttgcggaagatgaatgtggccttcgaggagcagaacgcggtactgcagaggcacacgcagagcatgagcagcgcgcgcgagcgtctggagcaggagctggcgctggaggagcggaggacgctggcgctgcagcagcagctccaggccgtgcgccaggcgctcaccgccagcttcgcctcactgccggtgccgggcacgggcgaaacgcccacgctgggcactctggacttctacatggcccggcttcacggagccatcgagcgcgaccccgcccagcacgagaagctcatcgtccgcatcaaggaaatcctggcccaggtcgccagcgagcacctgtga

SEQ ID NO: 35 - protein, 677 aa, synthetic construct
MAERKQSGKAAEDEEVPAFFKNLGSGSPKPRQKFCGMFCPVEGSSENKTIDFDSLSVGRGSGQVVAQQRDVAHLGPDPQPPYSRQGRRAGGEPSVESGRKVEIRRASGKEALQNINDQSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 36 - DNA, 2034 bp, synthetic construct
  source 1..2034: mol_type = other DNA
atggccgagagaaagcaatccgggaaggcggcagaggacgaagaggtccctgctttttttaaaaacctgggctccggcagccccaagccccggcagaaattctgtggcatgttctgcccggtggaagggtcctcggagaacaagaccatcgacttcgactcgctgtcggtgggccggggctcggggcaggtggtggctcagcagcgggacgtcgcccacttgggcccggacccgcagccgccgtactcgcggcagggccggcgcgccggcggagagccatctgttgaatcgggccggaaggtggagatccggagggcctcgggcaaagaagccctgcagaacatcaacgaccagagcgatcgtcttctgatcaaaggaggtaaaattgttaatgatgaccagtcgttctatgcagacatatacatggaagatgggttgatcaagcaaataggagaaaatctgattgtgccaggaggagtgaagaccatcgaggcccactcccggatggtgatccccggaggaattgacgtccacactcgtttccagatgcctgatcagggaatgacgtctgctgatgatttcttccaaggaaccaaggcggccctggctgggggaaccactatgatcattgaccacgttgttcctgagcctgggacaagcctgctcgctgcctttgaccagtggagggaatgggccgacagcaagtcctgctgtgactactctctgcatgtggacatcagtgagtggcataagggcatccaggaggagatggaagcgcttgtgaaggatcacggggtaaattccttcctcgtgtacatggctttcaaagatcgcttccagctaacggattgccagatttatgaagtactgagtgtgatccgggatattggcgccatagcccaagtccacgcagaaaatggcgacatcattgcagaggagcagcagaggatcctggatctgggcatcacgggccccgagggacatgtgctgagccgacctgaggaggtcgaggccgaagccgtgaatcgtgccatcaccatcgccaaccagaccaactgcccgctgtatatcaccaaggtgatgagcaaaagctctgctgaggtcatcgcccaggcacggaagaagggaactgtggtgtatggcgagcccatcactgccagcttgggaacggacggctcccattactggagcaagaactgggccaaggctgctgcctttgtcacctccccacccttgagccctgatccaaccactccagactttctcaactccttgctgtcctgtggagacctccaggtcacgggcagtgcccattgcacgtttaacactgcccagaaggctgtaggaaaggacaacttcaccctgattccggagggcaccaatggcactgaggagcggatgtccgtcatctgggacaaggctgtggtcactgggaagatggatgagaaccagtttgtggctgtgaccagcaccaatgcagccaaagtcttcaacctttacccccggaaaggccgcattgctgtgggatccgatgccgacctggtcatctgggaccccgacagcgttaaaaccatctctgccaagacacacaacagctctctcgagtacaacatctttgaaggcatggagtgccgcggctccccactggtggtcatcagccaggggaagattgtcctggaggacggcaccctgcatgtcaccgaaggctctggacgctacattccccggaagcccttccctgattttgtttacaagcgtatcaaggcaaggagcaggctggctgagctgagaggggttcctcgtggcctgtatgacggacctgtgtgtgaagtgtctgtgacgcccaagacagtcactccagcctcctcggccaagacgtctcctgccaagcagcaggccccacctgtccggaacctgcaccagtctggattcagtttgtctggtgctcagattgatgacaacattccccgccgcaccacccagcgtatcgtggcgccccccggtggccgtgccaacatcaccagcctgggctag

SEQ ID NO: 37 - protein, 387 aa, synthetic construct
MATVIPGPLSLGEDFYREAIEHCRSYNARLCAERSLRLPFLDSQTGVAQNNCYIWMEKTHRGPGLAPGQIYTYPARCWRKKRRLNILEDPRLRPCEYKIDCEAPLKKEGGLPEGPVLEALLCAETGEKKIELKEEETIMDCQKQQLLEFPHDLEVEDLEDDIPRRKNRAKGKAYGIGGLRKRQDTASLEDRDKPYVCDICGKRYKNRPGLSYHYTHTHLAEEEGEENAERHALPFHRKNNHKQFYKELAWVPEAQRKHTAKKAPDGTVIPNGYCDFCLGGSKKTGCPEDLISCADCGRSGHPSCLQFTVNMTAAVRTYRWQCIECKSCSLCGTSENDDQLLFCDDCDRGYHMYCLSPPMAEPPEGSWSCHLCLRHLKEKASAYITLT

SEQ ID NO: 38 - DNA, 1164 bp, synthetic construct
  source 1..1164: mol_type = other DNA
atggccactgtcatccctggccccctgagcctaggcgaggacttctaccgcgaggccatcgagcactgccgcagttacaacgcgcgcctgtgcgccgagcgcagcctgcgactgcccttcctcgactcgcagaccggcgtggcccagaacaactgctacatctggatggagaagacccaccgcgggccgggtttggccccgggacagatttacacgtaccccgcccgctgttggaggaagaaacggagactcaacatcctggaggaccccagactcaggccctgcgagtacaagatcgactgtgaagcacccctgaagaaggagggtggcctcccggaagggccggtcctcgaggctctactgtgtgcagagacgggggagaagaagattgagctgaaggaggaggagaccattatggactgtcagaaacagcagttgctggagtttccgcatgacctcgaggtggaagacttggaggatgacattcccaggaggaagaacagggccaaaggaaaggcatatggcatcgggggtctccggaaacgccaggacaccgcttccctggaggaccgagacaagccgtatgtctgtgatatctgtgggaaacggtataagaaccggccggggctcagctaccactacacccacacccacctggccgaggaggagggggaggagaacgccgaacgccacgccctgcccttccaccggaaaaacaaccataaacagttttacaaagaattggcctgggtccctgaggcacaaaggaaacacacagccaagaaggcgcccgacggcactgtcatccccaacggctactgtgacttctgcctggggggctccaagaagacggggtgtcccgaggacctcatctcctgtgcggactgtgggcgatcaggacacccctcgtgtttacaattcacggtgaacatgacggcagccgtgcggacctaccgctggcagtgcatcgagtgcaaatcctgcagcctgtgcggaacctccgagaacgacgaccagctgctgttttgtgatgactgcgatcggggttaccacatgtactgcctgagtccccccatggcggagcccccggaagggagctggagctgtcacctctgtctccggcacctgaaggaaaaggcttctgcttacatcaccctcacctag

SEQ ID NO: 39 - protein, 273 aa, synthetic construct
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 40 - DNA, 822 bp, synthetic construct
  source 1..822: mol_type = other DNA
atgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccagctcccttaggaatgacagcagtagcagtaataggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaa

SEQ ID NO: 41 - protein, 359 aa, synthetic construct
METQLSNGPTCNNTANGPTTINNNCSSPVDSGNTEDSKTNLIVNYLPQNMTQEELKSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGISRGVGFIRFDKRIEAEEAIKGLNGQKPPGATEPITVKFANNPSQKTNQAILSQLYQSPNRRYPGPLAQQAQRFRLDNLLNMAYGVKRFSPMTIDGMTSLAGINIPGHPGTGWCIFVYNLAPDADESILWQMFGPFGAVTNVKVIRDFNTNKCKGFGFVTMTNYDEAAMAIASLNGYRLGDRVLQVSFKTNKTHKA

SEQ ID NO: 42 - DNA, 1080 bp, synthetic construct
  source 1..1080: mol_type = other DNA
atggaaacacaactgtctaatgggccaacttgcaataacacagccaatggtccaaccaccataaacaacaactgttcgtcaccagttgactctgggaacacagaagacagcaagaccaacttaatagtcaactaccttcctcagaacatgacacaggaggaactaaagagtctctttgggagcattggtgaaatagagtcctgtaagcttgtaagagacaaaataacagggcagagcttgggatatggctttgtgaactacattgaccccaaggatgcagagaaagctatcaacaccctgaatggattgagacttcaaaccaaaacaataaaagtttcctatgctcgcccaagttcagcttctatcagagatgcaaatttatatgtcagcggacttccaaaaacaatgacccagaaggagttggaacagcttttttcacaatatggacgcattattacttctcgtattcttgtcgaccaggtcactggcatatcaaggggtgtagggtttattcgatttgacaagcgaattgaggcagaagaagctatcaaaggcctaaatggccagaaacctcccggtgccacggagccaatcactgtaaagtttgctaataacccaagccaaaaaaccaatcaggccatcctttcccagctgtaccagtctccaaacagaaggtatccaggaccgctagctcagcaggcacagcgttttaggttggacaatctgctcaatatggcttatggagtaaagaggttttctccaatgaccattgacggaatgaccagtttggctggaattaatatccctgggcaccctggaacagggtggtgtatatttgtgtacaacctggctcctgacgcagatgagagtatcctgtggcaaatgtttgggccttttggagctgtcaccaatgtgaaggtcatccgtgactttaacaccaataaatgcaaaggttttggatttgtgactatgacaaactatgatgaggctgccatggcgatagctagcctcaatggataccgtctgggagacagagtactgcaggtctcctttaagacaaacaaaacgcacaaagcctaa

SEQ ID NO: 43 - protein, 367 aa, synthetic construct
MVTQILGAMESQVGGGPAGPALPNGPLLGTNGATDDSKTNLIVNYLPQNMTQDEFKSLFGSIGDIESCKLVRDKITGQSLGYGFVNYSDPNDADKAINTLNGLKLQTKTIKVSYARPSSASIRDANLYVSGLPKTMSQKEMEQLFSQYGRIITSRILVDQVTGVSRGVGFIRFDKRIEAEEAIKGLNGQKPLGAAEPITVKFANNPSQKTGQALLTHLYQSSARRYAGPLHHQTQRFRLDNLLNMAYGVKSPLSLIARFSPIAIDGMSGLAGVGLSGGAAGAGWCIFVYNLSPEADESVLWQLFGPFGAVTNVKVIRDFTTNKCKGFGFVTMTNYDEAAMAIASLNGYRLGERVLQVSFKTSKQHKA

SEQ ID NO: 44 - DNA, 1104 bp, synthetic construct
  source 1..1104: mol_type = other DNA
atggtcactcagatactgggggccatggagtctcaggtgggggggggcccggccggcccggccctgcccaacgggccactccttggtacaaatggagccactgacgacagcaagaccaacctcatcgtcaactacctgccccagaacatgacccaggatgagttcaagagtctcttcggcagcattggcgacatcgagtcctgcaagttggttcgggacaagatcacagggcagagccttggctacgggtttgtgaactattctgaccccaatgatgcagacaaagccatcaacaccctcaacggcctcaaattacagacgaagaccatcaaggtgtcctatgccagacccagttcagcatccatccgggatgctaacctgtacgtcagcgggctccccaagaccatgagccagaaagagatggagcagctcttctcccagtacggccgcatcatcacgtcccgcatcctggtggaccaggtcacaggtgtctctcggggtgtgggattcatccgctttgacaagaggattgaggccgaagaggctatcaaaggactgaatgggcagaagccgctgggcgcagctgagcccatcacagtcaagttcgcgaacaacccaagtcagaagacggggcaggcgctgctcacccacctctaccagtcatccgcccggcgctacgcaggccccctacaccatcagacccagcgtttccggctggacaatttgctcaacatggcctacggcgtcaagagtcccctgtcgctcatcgccaggttctcgccgatcgccatcgatggtatgagcggcctggcgggcgtgggcctgtcggggggcgcggcgggcgccggctggtgcatcttcgtgtacaacctgtcaccggaggcagacgagagcgtgctgtggcagctgttcgggccttttggggcagtcaccaacgtcaaggtcatccgtgatttcaccaccaacaagtgcaagggtttcggcttcgtgaccatgaccaactatgacgaggcggccatggccatcgccagcctgaacggctatcgcctgggcgagcgcgtgctgcaggtctccttcaagaccagcaaacagcacaaggcgtga

SEQ ID NO: 45 - protein, 380 aa, synthetic construct
MVMIISTMEPQVSNGPTSNTSNGPSSNNRNCPSPMQTGATTDDSKTNLIVNYLPQNMTQEEFRSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGVSRGVGFIRFDKRIEAEEAIKGLNGQKPSGATEPITVKFANNPSQKSSQALLSQLYQSPNRRYPGPLHHQAQRFRLDNLLNMAYGVKRLMSGPVPPSACPPRFSPITIDGMTSLVGMNIPGHTGTGWCIFVYNLSPDSDESVLWQLFGPFGAVNNVKVIRDFNTNKCKGFGFVTMTNYDEAAMAIASLNGYRLGDRVLQVSFKTNKAHKS

SEQ ID NO: 46 - DNA, 1143 bp, synthetic construct
  source 1..1143: mol_type = other DNA
atggttatgataattagcaccatggagcctcaggtgtcaaatggtccgacatccaatacaagcaatggaccctccagcaacaacagaaactgtccttctcccatgcaaacaggggcaaccacagatgacagcaaaaccaacctcatcgtcaactatttaccccagaatatgacccaagaagaattcaggagtctcttcgggagcattggtgaaatagaatcctgcaaacttgtgagagacaaaattacaggacagagtttagggtatggatttgttaactatattgatccaaaggatgcagagaaagccatcaacactttaaatggactcagactccagaccaaaaccataaaggtctcatatgcccgtccgagctctgcctcaatcagggatgctaacctctatgttagcggccttcccaaaaccatgacccagaaggaactggagcaacttttctcgcaatacggccgtatcatcacctcacgaatcctggttgatcaagtcacaggagtgtccagaggggtgggattcatccgctttgataagaggattgaggcagaagaagccatcaaagggctgaatggccagaagcccagcggtgctacggaaccgattactgtgaagtttgccaacaaccccagccagaagtccagccaggccctgctctcccagctctaccagtcccccaaccggcgctacccaggtccacttcaccaccaggctcagaggttcaggctggacaatttgcttaatatggcctatggcgtaaagagactgatgtctggaccagtccccccttctgcttgtccccccaggttctccccaattaccattgatggaatgacaagccttgtgggaatgaacatccctggtcacacaggaactgggtggtgcatctttgtctacaacctgtcccccgattccgatgagagtgtcctctggcagctctttggcccctttggagcagtgaacaacgtaaaggtgattcgtgacttcaacaccaacaagtgcaagggattcggctttgtcaccatgaccaactatgatgaggcggccatggccatcgccagcctcaacgggtaccgcctgggagacagagtgttgcaagtttcctttaaaaccaacaaagcccacaagtcctga

SEQ ID NO: 47 - protein, 543 aa, synthetic construct
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPLKWPNPENFDPARFLDKDGLINKDLTSRVMIFSVGKRRCIGEELSKMQLFLFISILAHQCDFRANPNEPAKMNFSYGLTIKPKSFKVNVTLRESMELLDSAVQNLQAKETCQ

SEQ ID NO: 48 - DNA, 1632 bp, synthetic construct
  source 1..1632: mol_type = other DNA
atgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccactgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaataa

SEQ ID NO: 49 - protein, 605 aa, synthetic construct
MDPAEAVLQEKALKFMCSMPRSLWLGCSSLADSMPSLRCLYNPGTGALTAFQNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 50 - DNA, 1818 bp, synthetic construct
  source 1..1818: mol_type = other DNA
atggacccggccgaggcggtgctgcaagagaaggcactcaagtttatgtgctctatgcccaggtctctgtggctgggctgctccagcctggcggacagcatgccttcgctgcgatgcctgtataacccagggactggcgcactcacagctttccagaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagataa

SEQ ID NO: 51 - RNA, 33 nt, synthetic construct
  source 1..33: mol_type = other RNA
cattggcagaaacagcctccacatcttcttgag

SEQ ID NO: 52 - RNA, 30 nt, synthetic construct
  source 1..30: mol_type = other RNA
cgacttctccacatcgtgcttatgccacag

SEQ ID NO: 53 - RNA, 30 nt, synthetic construct
  source 1..30: mol_type = other RNA
catcagtgctcttccctgatggcacattag

SEQ ID NO: 54 - RNA, 30 nt, synthetic construct
  source 1..30: mol_type = other RNA
agaacccggattgttgccagattggtctct

SEQ ID NO: 55 - RNA, 29 nt, synthetic construct
  source 1..29: mol_type = other RNA
gtcctctgcaccgtccccgtggttgtgcg

SEQ ID NO: 56 - RNA, 31 nt, synthetic construct
  source 1..31: mol_type = other RNA
ctccgacatgttatctggacggatggagcgc

SEQ ID NO: 57 - RNA, 29 nt, synthetic construct
  source 1..29: mol_type = other RNA
ttgacacttggatcccagaacacccgatc

SEQ ID NO: 58 - RNA, 31 nt, synthetic construct
  source 1..31: mol_type = other RNA
aaggactaatatgcagtttgaagtctggtcc

SEQ ID NO: 59 - RNA, 27 nt, synthetic construct
  source 1..27: mol_type = other RNA
acgtggagtgagtctaggaggggggat

SEQ ID NO: 60 - RNA, 29 nt, synthetic construct
  source 1..29: mol_type = other RNA
atttgacatgcattttgtaggtatgatcc

SEQ ID NO: 61 - RNA, 31 nt, synthetic construct
  source 1..31: mol_type = other RNA
atgttgccatagtaacgggcatctatgcagt

SEQ ID NO: 62 - RNA, 31 nt, synthetic construct
  source 1..31: mol_type = other RNA
tacacagttcatacatatccaacataagagg

SEQ ID NO: 63 - RNA, 30 nt, synthetic construct
  source 1..30: mol_type = other RNA
aggaggcgtttcctggcacgttgacgccag

SEQ ID NO: 64 - RNA, 30 nt, synthetic construct
  source 1..30: mol_type = other RNA
atattccttgaaatccttggcatgctggag

SEQ ID NO: 65 - RNA, 31 nt, synthetic construct
  source 1..31: mol_type = other RNA
tgttgcactgtgctttgcagcggcatcccgg

SEQ ID NO: 66 - RNA, 31 nt, synthetic construct
  source 1..31: mol_type = other RNA
ctgtgttcttcctgaacttctggctggcagc

SEQ ID NO: 67 - RNA, 30 nt, synthetic construct
  source 1..30: mol_type = other RNA
gatctgctcgcgccgctcgttcaggaagcg

SEQ ID NO: 68 - DNA, 33 bp, synthetic construct
  source 1..33: mol_type = other DNA
ctcaagaagatgtggaggctgtttctgccaatg

SEQ ID NO: 69 - DNA, 30 bp, synthetic construct
  source 1..30: mol_type = other DNA
ctgtggcataagcacgatgtggagaagtcg

SEQ ID NO: 70 - DNA, 30 bp, synthetic construct
  source 1..30: mol_type = other DNA
ctaatgtgccatcagggaagagcactgatg

SEQ ID NO: 71 - DNA, 30 bp, synthetic construct
  source 1..30: mol_type = other DNA
agagaccaatctggcaacaatccgggttct

SEQ ID NO: 72 - DNA, 29 bp, synthetic construct
  source 1..29: mol_type = other DNA
cgcacaaccacggggacggtgcagaggac

SEQ ID NO: 73 - DNA, 31 bp, synthetic construct
  source 1..31: mol_type = other DNA
gcgctccatccgtccagataacatgtcggag

SEQ ID NO: 74 - DNA, 29 bp, synthetic construct
  source 1..29: mol_type = other DNA
gatcgggtgttctgggatccaagtgtcaa

SEQ ID NO: 75 - DNA, 31 bp, synthetic construct
  source 1..31: mol_type = other DNA
ggaccagacttcaaactgcatattagtcctt

SEQ ID NO: 76 - DNA, 27 bp, synthetic construct
  source 1..27: mol_type = other DNA
atcccccctcctagactcactccacgt

SEQ ID NO: 77 - DNA, 29 bp, synthetic construct
  source 1..29: mol_type = other DNA
ggatcatacctacaaaatgcatgtcaaat

SEQ ID NO: 78 - DNA, 31 bp, synthetic construct
  source 1..31: mol_type = other DNA
actgcatagatgcccgttactatggcaacat

SEQ ID NO: 79 - DNA, 31 bp, synthetic construct
  source 1..31: mol_type = other DNA
cctcttatgttggatatgtatgaactgtgta

SEQ ID NO: 80 - DNA, 30 bp, synthetic construct
  source 1..30: mol_type = other DNA
ctggcgtcaacgtgccaggaaacgcctcct

SEQ ID NO: 81 - DNA, 30 bp, synthetic construct
  source 1..30: mol_type = other DNA
ctccagcatgccaaggatttcaaggaatat

SEQ ID NO: 82 - DNA, 31 bp, synthetic construct
  source 1..31: mol_type = other DNA
ccgggatgccgctgcaaagcacagtgcaaca

SEQ ID NO: 83 - DNA, 31 bp, synthetic construct
  source 1..31: mol_type = other DNA
gctgccagccagaagttcaggaagaacacag

SEQ ID NO: 84 - DNA, 30 bp, synthetic construct
  source 1..30: mol_type = other DNA
cgcttcctgaacgagcggcgcgagcagatc

SEQ ID NO: 85 - DNA, 22 bp, synthetic construct
  source 1..22: mol_type = other DNA
gtctcctctgacttcaacagcg

SEQ ID NO: 86 - DNA, 22 bp, synthetic construct
  source 1..22: mol_type = other DNA
accaccctgttgctgtagccaa

SEQ ID NO: 87 - DNA, 20 bp, synthetic construct
  source 1..20: mol_type = other DNA
ggagagcgaggatgaactgg

SEQ ID NO: 88 - DNA, 20 bp, synthetic construct
  source 1..20: mol_type = other DNA
gcattggcagaaacagcctc

SEQ ID NO: 89 - DNA, 20 bp, synthetic construct
  source 1..20: mol_type = other DNA
acccaaccactgtgtgtcag

SEQ ID NO: 90 - DNA, 20 bp, synthetic construct
  source 1..20: mol_type = other DNA
cagcgacttctccacatcgt

SEQ ID NO: 91 - DNA, 20 bp, synthetic construct
  source 1..20: mol_type = other DNA
cagaacaaggaacccaggct

SEQ ID NO: 92 - DNA, 20 bp, synthetic construct
  source 1..20: mol_type = other DNA
gagtggcaataggggctgtt

SEQ ID NO: 93 - DNA, 20 bp, synthetic construct
  source 1..20: mol_type = other DNA
cccaagtgtacaggacggac

SEQ ID NO: 94 - DNA, 20 bp, synthetic construct
  source 1..20: mol_type = other DNA
aacccggattgttgccagat

SEQ ID NO: 95 - DNA, 20 bp, synthetic construct
  source 1..20: mol_type = other DNA
gacggtgcagaggacttcaa

SEQ ID NO: 96 - DNA, 20 bp, synthetic construct
  source 1..20: mol_type = other DNA
tgttcagctgctccttctgg

SEQ ID NO: 97 - DNA, 20 bp, synthetic construct
  source 1..20: mol_type = other DNA
ctatcgccctcacaaagcca

SEQ ID NO: 98 - DNA, 20 bp, synthetic construct
  source 1..20: mol_type = other DNA
ctgcttgctgtactccgaca

SEQ ID NO: 99 - DNA, 20 bp, synthetic construct
  source 1..20: mol_type = other DNA
atttgttcgggcatgttggc

SEQ ID NO: 100 - DNA, 20 bp, synthetic construct
  source 1..20: mol_type = other DNA
cggccaacaatcacatcgtc

(19) United States
(12) Patent Application Publication
(10) Pub. No.: US 2026/0085284 A1
(43) Pub. Date: Mar. 26, 2026

(54) METHOD FOR TRANS-DIFFERENTIATING NON-NEURONAL CELLS INTO NEURONS AND USE THEREOF

(71) Applicant: SHANGHAI GENEMAGIC BIOSCIENCES CO., LTD., Shanghai (CN)

(72) Inventors: Haibo ZHOU, Shanghai (CN); Xinde HU, Shanghai (CN); Jinlin SU, Shanghai (CN)

(73) Assignee: SHANGHAI GENEMAGIC BIOSCIENCES CO., LTD., Shanghai (CN)

(21) Appl. No.: 19/107,933

(22) PCT Filed: Aug. 30, 2023
(86) PCT No.: PCT/CN2023/115930

§ 371 Date: Feb. 28, 2025

(30) Foreign Application Priority Data
Aug. 30, 2022 (WO) PCT/CN2022/115708

(51) Int. Cl. C12N 5/0793; A61P 27/02; C12N 9/22; C12N 15/11; C12N 15/86; C12N 15/90

(52) U.S. Cl. CPC C12N 5/0619; A61P 27/02; C12N 9/226; C12N 15/11; C12N 15/86; C12N 15/907; C12N 2310/20; C12N 2506/08; C12N 2510/00; C12N 2750/14143

(57) ABSTRACT

Provided is a method for trans-differentiating non-neuronal cells of a mammal into neurons. The method comprises: providing a negative regulator capable of reducing the expression of negative regulatory genes, the negative regulatory gene including RCOR1, RCOR2, RCOR3, Sin3a, Sin3b, HDAC1, HDAC2, KDM1A, PHF21A, BAF53a, G9a, USP14, HuR, BrG1, EZH2, CDYL, or HMG20B; and/or providing a positive regulator capable of improving the expression of positive regulatory genes, the positive regulatory genes including DPYSL2, BAF45b, SCF, HuB, HuC, HuD, CYP1B1, or BTRC. An effective amount of the negative regulator or the positive regulator contacting the non-neuronal cells can induce trans-differentiation of the non-neuronal animal cells into neurons.

This application claims the priority of the patent with the application number PCT/CN2022/115708, which was submitted on Aug. 30, 2022.

### TECHNICAL FIELD

The present application relates to the field of translational medicine, and more specifically, to a method of transdifferentiating non-neuronal cells into neurons, and the use of this method for preparing medicaments for the treatment or prevention of diseases related to neuronal injury or neuronal death.

### BACKGROUND TECHNOLOGY

The main pathological changes caused by nervous system damage and various neurodegenerative diseases are neuronal degeneration, necrosis and neuronal injury. Due to the very limited self-repair ability of the nervous system (such as the brain, spinal cord, optic nerve, etc.), it is difficult to repair neuron cells autonomously. Diseases associated with neuronal function loss or neuronal death have always been difficult to treat, such as Parkinson's disease, schizophrenia, depression, Alzheimers disease, Huntington's disease, sleep disorders, brain trauma, stroke, visual system diseases associated with the dysfunction or death of RGC or photoreceptor cell, blindness, deafness, etc., currently, the treatment methods for these diseases can only alleviate the disease progression, but have no effective therapeutic means.

Cell trans-differentiation refers to the process by which one type of differentiated cells can transform into another type of differentiated cells in terms of structure and function under certain conditions. If some non-neuronal cells in the nervous system can be reprogrammed into neuronal cells, it would be expected to fundamentally treat the diseases associated with neuronal function loss or neuronal death.

Recent studies have shown that regulating the expression of certain genes in the nervous system can guide non-neuronal cells to transform into neurons. For example, inhibiting PTBP1 can induce the trans-differentiation of glial cells into neurons. However, these studies are in the early stages, and the field still lacks effective means of transdifferentiating non-neuronal cells into neurons.

### Contents of the Invention

One object of this disclosure is to provide a method for generating neuronal cells from glial cells in vivo.

Another object of this disclosure is to provide the use of inhibitors capable of inhibiting the expression or activity of one or more genes or their RNAs or their encoded proteins selected from the following: RCOR1, RCOR2, RCOR3, Sin3a, Sin3b, HDAC1, HDAC2, KDM1A, PHF21A, BAF53a, G9a, USP14, HuR, BrG1, EZH2, CDYL, HMC20B, the inhibitors are used for preventing and/or treating the diseases associated with neuronal function loss or neuronal death.

Another object of this disclosure is to provide the use of agonists capable of enhancing the expression or activity of one or more genes or their RNAs or their encoded proteins selected from the following: DPYSL2, BAP45b, SCF, HuB, HuC, HuD, CYP1B1, BTRC, the agonists are used for preventing and/or treating the diseases associated with neuronal function loss or neuronal death.

In one embodiment, this disclosure provides a method for generating neuronal cells from glial cells, the method comprises transdifferentiating or reprogramming the glial cells into neurons by utilizing one or more inhibitors of the genes or their RNAs or their encoded protein selected from the following: RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B, wherein the inhibitor reduces the expression or activity of the gene or its RNA or its encoded protein, the method includes: (i) providing the inhibitor,
(ii) providing the said glial cells;
(iii) bringing the inhibitor in an amount effective to induce glial cells transdifferentiating or reprogramming into neuronal cells to contact with the glial cells; and
(iv) optionally, identifying and/or isolating the transdifferentiated or reprogrammed cells.

In one embodiment, this disclosure provides a method for generating neuronal cells from glial cells, the method comprises transdifferentiating or reprogramming the glial cells into neurons by utilizing one or more agonists of the genes or their RNAs or their encoded protein selected from the following: DPYSL2, BAF45b, SCF, HuB, HuC, HuD, CYP1B1, BTRC, wherein the agonist increases the expression or activity of the gene or its RNA or its encoded protein, the method includes:
(i) providing the said agonist;
(ii) providing the said glial cells;
(iii) bringing the agonist in an amount effective to induce glial cells transdifferentiating or reprogramming into neuronal cells to contact with the glial cells; and
(iv) optionally, identifying and/or isolating the transdifferentiated or reprogrammed cells.

In a preferred embodiment, the glial cells are mammalian glial cells, including the glial cells from human, non-human primate, mouse, and rat.

In a more preferred embodiment, the mammalian glial cells include astrocytes, oligodendrocytes, microglia, NG2 cells, Müller glial cells, glioma cells or spiral ganglion glial cells.

In a more preferred embodiment, the glial cells are astrocytes or muller glial cells.

In a preferred embodiment, the astrocytes are derived from the brain, midbrain, cerebellum, brainstem, or spinal cord.

In a more preferred embodiment, the astrocytes are derived from striatum or substantia nigra.

In a more preferred embodiment, the Müller glial cells are derived from retina.

In a more preferred embodiment, the spiral ganglion glial cells are derived from the inner ear or vestibulum.

In a preferred embodiment, the neuronal cells are mammalian neurons, including neurons of humans, non-human primates, rats, and mice.

In a more preferred embodiment, wherein the said neuronal cells select from the group consisting of dopamine neurons, 5-HT neurons, NE neurons, ChAT neurons, motor neurons, GABA neurons, glutamatergic neurons, spinal cord neurons, spinal motor neurons, spinal sensory neurons, photoreceptor cells (rods and cones), bipolar cells, amacrine cells, retinal ganglion cells (RGC), cochlear nerve cells, pyramidal neurons, interneurons, medium spiny neurons (MSN), Purkinje cells, granule cells, olfactory receptor neurons, periglomerular cells, or the combinations thereof, more preferably, the said neuronal cells select from dopamine neurons, retinal ganglion cells and photoreceptor cells.

In a more preferred embodiment, wherein the said glial cells are astrocytes; and the said neuronal cells are dopamine neurons.

In a more preferred embodiment, wherein the said glial cells are Müller glial cells; and the said neuronal cells are RGC or photoreceptor cells.

In another embodiment, this disclosure provides the use of inhibitors of one or more genes or their RNAs or their encoded proteins selected from the following: RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B, which is used to prepare a medicament, the medicament is used to prevent and/or treat diseases related to neuronal function loss or neuronal death, wherein, the inhibitor reduces the expression or activity of the gene or its RNA or its encoded protein.

In another embodiment, this disclosure provides the use of agonists of one or more genes or their RNA or their encoded proteins selected from the following: DPYSL2, BAP45b, SCP, HuB, HuC, HuD, CYP1B1, BTRC, which is used to prepare a medicament, the medicament is used to prevent and/or treat diseases related to neuronal function loss or neuronal death, wherein, the agonist increases the expression or activity of the gene or its RNA or its encoded protein.

In a preferred embodiment, the said medicament is formulated for in vivo administration to the nervous system, visual system, and auditory system, including the striatum, substantia nigra, midbrain ventral tegmental area, spinal cord, hypothalamus, dorsal midbrain, cerebral cortex, hippocampus, cerebellum, subretina, vitreous cavity, inner ear cochlea and vestibulum.

In a more preferred embodiment, the medicament is formulated for administration into the striatum, substantia nigra, subretina and vitreous cavity.

In a preferred embodiment, the disease associated with neuronal function loss or death is a neurological disease, including Parkinson's disease, schizophrenia, depression, Alzheimer's disease, Huntington's disease, epilepsy, sleep disorder, ataxia, PloyQ disease, cerebral ischemia, brain injury, addiction, motor neuron disease, amyotrophic lateral sclerosis, spinal muscular atrophy, Pick's disease, visual system diseases associated with function loss or death of RGC or photoreceptor cells, blindness, and deafness.

In a more preferred embodiment, the diseases related to neuronal function loss or death are Parkinson's disease and visual system diseases associated with function loss or death of RGC or photoreceptor cells.

In another embodiment, this disclosure provides a method of generating retinal ganglion cells (RGC) or photoreceptor cells from Müller glial cells, which comprise transdifferentiating or reprogramming the Müller glial cells into RGC or photoreceptor cells by using one or more inhibitors of the expression or activity of the genes or their RNAs or their encoded protein selected from the following: RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B, wherein the inhibitor reduces the expression or activity the gene or its RNA or its encoded protein.

In another embodiment, this disclosure provides a method of generating retinal ganglion cells (RGC) or photoreceptor cells from Müller glial cells, which comprise transdifferentiating or reprogramming the Müller glial cells into RGC or photoreceptor cells by using one or more agonists of the expression or activity of a genes or their RNAs or their encoded protein selected from the following: DPYSL2, BAF45b, SCF, HuB, HuC, HuD, CYP1B1, BTRC, wherein the agonist increases the expression or activity of the gene or its RNA or its encoded protein.

In a preferred embodiment, the Müller glial cells are derived from the retina.

In a preferred embodiment, the photoreceptor cells include rods and cones.

In another embodiment, this disclosure provides the use of inhibitors capable of inhibiting the expression or activity of one or more genes or their RNAs or their encoded proteins selected from the following: RCOR1, RCOR2, RCOR3, Sin3a, Sin3b, HDAC1, HDAC2, KDM1A, PHF21A, BAF53a, (39a, USP14, HuR, BrG1, EZH2, CDYL, HMG20B, which is used to prepare a medicament, the medicament is used to prevent and/or treat the visual system diseases associated with the function loss or death of RGC or photoreceptor cell, wherein, the inhibitor reduces the expression or activity of the gene or its RNA or its encoded protein.

In another embodiment, this disclosure provides the use of agonists capable of enhancing the expression or activity of one or more genes or their RNAs or their encoded proteins selected from the following: DPYSL2, BAF45b, SCF, HuB, HuC, HuD, CYP1B1, BTRC, which is used to prepare a medicament, the medicament is used to prevent and/or treat visual system diseases related to the function loss or death of RGC or photoreceptor cell, wherein, the agonist increases the expression or activity the gene or its RNA or its coding protein.

In a preferred embodiment, the medicament is formulated for administration to the visual system.

In a more preferred embodiment, the medicament is formulated for administration into subretina or vitreous cavity, wherein the medicament exerts its effect by acting on Müller glial cells.

In a preferred embodiment, the neurological disease associated with RGC function loss or death selects from: vision impairment caused by RGC cell death, glaucoma, age-related RGC pathology, diabetes-related retinopathy, optic nerve injury, retinal ischemia or hemorrhage, Leber's hereditary optic neuropathy, or combinations thereof.

In a preferred embodiment, the visual system disease associated with photoreceptor cell function loss or death selects from: photoreceptor cell degeneration or death caused by injury or degenerative disease, macular degeneration, retinitis pigmentosa, diabetes-related blindness, nyctalopia, color blindness, hereditary blindness, congenital amaurosis, or combinations thereof.

In a preferred embodiment, the inhibitor or agonist selects from: antibody, small molecule compound, mRNA, microRNA, siRNA, shRNA, antisense oligonucleotide, binding protein or protein domain, polypeptide, nucleic acid adapter, gene editor, PROTAC, expression vector containing promoter, endogenous expression activator, protein analog or protein enhancer, synthetically produced or modified versions of the aforementioned inhibitors or agonists, or combinations thereof.

In another embodiment, this disclosure provides a pharmaceutical composition or medicine kit or reagent kit, which comprise inhibitors capable of inhibiting the expression or activity of one or more genes or their RNAs or their encoded proteins selected from the following: RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B; and/or comprise one or more agonists capable of enhancing the expression or activity of the genes or their RNAs or their encoded proteins selected from the following: DPYSL2, BAF45b, SCF, HuB, HuC, HuD), CYP1B1, BTRC.

In a preferred embodiment, the inhibitor comprises:
(a) gene editing protein or its expression vector, and
(b) one or more gRNAs or the expression vectors thereof, wherein the gRNA is DNA or RNA that guides the gene editing protein to specifically bind to the said gene.

In a preferred embodiment, the editing system includes: CRISPR system (including CRISPR/dCas system), ZFN system, TALEN system, RNA editing system, or a combination thereof.

In a preferred embodiment, the gene editing protein is an RNA-targeting gene editing protein.

In a preferred embodiment, the gRNA is an RNA-targeting gRNA.

In a preferred embodiment, wherein said agonist comprises: an expression vector containing a promoter, an endogenous expression activator, a protein analog or a protein enhancer.

In a preferred embodiment, the pharmaceutical composition or medicine kit or reagent kit further comprises a carrier for delivering the inhibitor.

In a preferred embodiment, the carrier is a viral vector, liposome, nanoparticle, exosome, virus-like particle, preferably AAV.

In a preferred embodiment, the RNA-targeting gene editing protein selects from: Cas13d, Cas13e, Cas13a, Cas13b, Cas13c, Cas13f, and their functional domains.

In a more preferred embodiment, the RNA-targeting gene editing protein select from: CasRx, Cas13e, and Cas13f.

In a more preferred embodiment, the RNA-targeting gene editing protein is CasRx.

In a preferred embodiment, said pharmaceutical composition or medicine kit or reagent kit contains only a single type of gRNA targeting said mRNA sequence, or contains 2, 3, 4, 5, 6, 7, 8, 9, 10, 11, or 12 different gRNAs targeting said mRNA sequence.

In another preferred embodiment, the gRNA expression vector encodes gRNA comprising only a single type of gRNA targeting the nRNA sequence or comprising 2, 3, 4, 5, 6, 7, 8, 9, 10, 11, 12 different gRNAs.

In a preferred embodiment, the expression vector contains:
i) a nucleotide sequence encoding the gene editing protein, the nucleotide sequence is operably linked to a promoter bringing the expression of the gene editing protein, and
ii) at least one nucleotide sequence encoding a gRNA targeting the said mRNA or DNA sequence, the nucleotide sequence is operably linked to a promoter bringing the expression of said gRNA in a mammalian cell, such as the U6 promoter.

In a preferred embodiment, the promoter is a broad-spectrum promoter or a specific promoter.

In a more preferred embodiment, the broad-spectrum promoter selects from CMV, CBH, CAG, POK, SV40, EF1A, EFS, and pGlobin.

In a more preferred embodiment, the specific promoter is a glial cell-specific promoter or a Müller glial (MG) cell-specific promoter.

In a more preferred embodiment, the glial cell-specific promoter selects from GFAP promoter, ALDH1L1 promoter, EAAT1/GLAST promoter, glutamine synthetase promoter, S100β promoter and EAAT2/GLT-1 promoter, NG2 promoter, CD68 promoter, F4/80 promoter, or the MG cell-specific promoter selects from GFAP promoter, ALDH1L1 promoter. Glast (also known as Slc1a3) promoter and Rlbp1 promoter.

In a preferred embodiment, the expression vector is contained in a viral particle.

In a preferred embodiment, the expression vector is gene therapy vector.

In a more preferred embodiment, the gene therapy vector is a viral gene therapy vector.

In a more preferred embodiment, the expression vector is a viral vector selected from: adeno-associated virus (AAV) vector, recombinant adeno-associated virus vector (rAAV), self-complementing adeno-associated virus vector (scAAV), adenovirus vector, lentiviral vector, retroviral vector, herpesvirus vector, SV40 vector, poxvirus vector, and combinations thereof.

In a more preferred embodiment, the expression vector is AAV or rAAV.

In a preferred embodiment, the composition is locally administrated to at least one of the following: i) glial cells in the retina; ii) glial cells in the striatum, preferably glial cells in the putamen; iii) glial cells in substantia nigra; iv) glial cells in inner ear; v) glial cells in spinal cord; vi) glial cells in prefrontal cortex; vii) glial cells in motor cortex; viii) glial cells in thalamus; ix) glial cells in ventral tegmental area (VTA); x) glial cells in hippocampus; xi) glial cells in cerebellum; and xii) glial cells in brainstem.

In a preferred embodiment, the composition further comprises i) one or more neuron-related factors, or ii) at least one expression vector for expressing one or more neuron-related factors in the glial cells.

In a more preferred embodiment, the one or more neuron-related factors select from: AscL1, Mytl1, Ngn1, Ngn2, NeuroD1, NeuroD2, NeuroG1, Pax6, Ptbp1, P53, Zic1, Ctdsp1, miR-9, miR-9-9*, miR-124, miR-124-124*, Let-7, Let-7b, miR-132, NeuroG2, Bm2, NeuroD4, Insm1, Prox1, FoxG1, Lhx6, Bcl2, Dlx1, Dlx2, Tlx3, Gata2, Gata3, Sox11, Lhx3, IsL1, etc.

In a preferred embodiment, said pharmaceutical composition or medicine kit or reagent kit further comprises i) one or more dopamine neuron-associated factors, or ii) at least one expression vector for expressing one or more dopamine neuron-related factors in said glial cells.

In a preferred embodiment, the composition is also formulated for cell transfection, cell infection, cellular endocytosis, injection, intracranial administration, spinal cord administration, intraocular administration, intra-aural administration, inhalation, parenteral administration, intravenous administration, intramuscular administration, subcutaneous administration, epidermal administration, or oral administration, as well as for inducing cells to transdifferentiate or reprogram in vitro, and then the cells transdifferentiated or reprogrammed in vitro are transplanted into the body.

In a preferred embodiment, the AAV vector contains:
i) a nucleotide sequence encoding the gene editing protein, the nucleotide sequence is operably linked to a promoter bringing the expression of the gene editing protein in glial cells; and
ii) at least one nucleotide sequence encoding a gRNA targeting the said mRNA or DNA sequence, the nucleotide sequence is operably linked to a promoter bringing the expression of said gRNA in a mammalian cell, such as the U6 promoter.

In a preferred embodiment, the promoter is a broad-spectrum promoter or a specific promoter.

In a more preferred embodiment, the broad-spectrum promoter selects from CMV, CBH, CAG, PGK, SV40, EF1A, EFS, and pGlobin.

In a more preferred embodiment, the specific promoter is a glial cell-specific promoter or a Müller glial (MG) cell-specific promoter.

In a more preferred embodiment, the glial cell-specific promoter selects from GFAP promoter, ALDH1L1 promoter, EAAT1/GLAST promoter, glutamine synthetase promoter, S100β promoter and EAAT2/GLT-1 promoter, NG2 promoter, CD68 promoter, F4/80 promoter.

In a more preferred embodiment, the MG cell-specific promoter selects from the group consisting of GFAP promoter, ALDH1L1 promoter, Glast (also known as Slc1a3) promoter and Rlbp1 promoter.

In a preferred embodiment, the trans-differentiation efficiency of glial cells is at least 0.1%, or at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, or the higher.

In another preferred embodiment, the disease associated with neuronal function loss or death selects from: Parkinson's disease, schizophrenia, depression, vision impairment caused by RGC cell death, diabetes-related retinopathy, glaucoma, age-related RGC pathology, optic nerve injury, retinal ischemia or hemorrhage, Leber's hereditary optic neuropathy, photoreceptor cell degeneration or death caused by injury or degenerative disease, macular degeneration, retinitis pigmentosa, diabetes-related blindness, nyctalopia, color blindness, hereditary blindness, congenital amaurosis, or combinations thereof.

In another preferred embodiment, the RGC can be integrated into the visual pathway and improve visual function.

In another preferred embodiment, the RGC can achieve functional projection to the central visual area and improve visual function.

In another preferred embodiment, said improve visual function refers to enhancing the visual function in a mammal suffering from a retinal disease caused by neurodegeneration.

In another preferred embodiment, while the MG cells are transdifferentiated into RGC cells, they are also differentiated into amacrines.

In another preferred embodiment, a number of glial cells in the striatum are reprogrammed or transdifferentiated, and at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or at least 30% of glial cells are converted into dopamine neurons.

In another preferred embodiment, the mammal includes a mammal suffering from a disease associated with neuronal function loss or death.

In another preferred embodiment, the mammal includes a human or non-human mammal.

In another preferred embodiment, the non-human mammal includes rodents (such as mice, rats, or rabbits), primates (such as monkeys).

In another preferred embodiment, the gene editor is driven by a glial cell-specific promoter (e.g., the GFAP promoter).

In another preferred embodiment, the gene editor includes one or more gRNA and gene editing protein.

In another preferred embodiment, the gRNA guides the gene editing protein to specifically bind to the RNA of the said gene.

In another preferred embodiment, the gRNA guides the gene editing protein to specifically bind to the mRNA of the said gene.

In another preferred embodiment, the nucleotide sequence of the gKNA is shown in Table 1 below.

TABLE 1
Nucleotide sequence and target sequence of gRNA

| | SEQ | | SEQ | |
|---|---|---|---|---|
| GENE | ID NO. | gRNA sequence | ID NO. | Target sequence |
| human- | 51 | cauuggcagaaacagccuccacaucuuc | 68 | ctcaagaagatgtggaggctgtttctgc |
| RCOR1 | | uugag | | caatg |
| human- | 52 | cgacuucuccacaucgugcuuaugccac | 69 | ctgtggcataagcacgatgtggagaag |
| RCOR2 | | ag | | tcg |
| human- | 53 | caucagugcucuucccugauggcacau | 70 | ctaatgtgccatcagggaagagcactg |
| RCOR3 | | uag | | atg |
| human- | 54 | agaacccggauuguugccagauugguc | 71 | agagaccaatctggcaacaatccgggt |
| Sin3a | | ucu | | tct |
| hUman- | 55 | guccucugcaccguccccgugguugug | 72 | cgcacaaccacggggacggtgcaga |
| Sin3b | | cg | | ggac |
| haman- | 56 | cuccgacauguuaucuggacggaugga | 73 | gcgctccatccgtccagataacatgtcg |
| HDAC1 | | gcgc | | gag |
| human- | 57 | uugacacuuggaucccagaacacccgau | 74 | gatcgggtgttctgggatccaagtgtca |
| KDM1A | | c | | a |
| HDAC2 | 58 | aaggacuaauaugcaguuugaagucug | 75 | ggaccagacttcaaactgcatattagtc |
| | | gucc | | ctt |
| PHF21A | 59 | acguggagugagucuaggaggggggau | 76 | atcccccctcctagactcactccacgt |
| BAF53A | 60 | auuugacaugcauuuuguagguaugau | 77 | ggatcatacctacaaaatgcatgtcaaa |
| | | cc | | t |
| G9a | 61 | auguugccauaguaacgggcaucuaug | 78 | actgcatagatgcccgttactatggcaa |
| | | cagu | | cat |
| USP14 | 62 | uacacaguucauacauauccaacauaag | 79 | cctcttatgttggatatgtatgaactgtgt |
| | | agg | | a |
| HuR | 63 | aggaggcguuuccuggcacguugacgc | 80 | ctggcgtcaacgtgccaggaaacgcct |
| | | cag | | cct |
| BRG1 | 64 | auauuccuugaaauccuuggcaugcug | 81 | ctccagcatgccaaggatttcaaggaat |
| | | gag | | at |
| EZH2 | 65 | uguugcacugugcuuugcagcggcauc | 82 | ccgggatgccgctgcaaagcacagtg |
| | | ccgg | | caaca |
| CDYL | 66 | cuguguucuuccugaacuucuggcugg | 83 | gctgccagccagaagttcaggaagaa |
| | | cagc | | cacag |
| HMG20B | 67 | gaucugcucgcgccgcucguucaggaa | 84 | cgcttcctgaacgagcggcgcgagca |
| | | gcg | | gatc |

As used herein, the letter “u” may be interchangeable with the letter “t” in the context of RNA sequences, which represent uridine or uridylic acid.

In another preferred embodiment, the source of the gene editing protein selects from: Streptococcus pyogenes, Staphylococcus aureus, Acidaminococcus sp, Lachnospiraceae bacterium, Ruminococcus flavefaciens, or combinations thereof.

In another preferred embodiment, the gene or its RNA or its encoded protein is from mammal, preferably, from human, monkey, mouse, rat, or rabbit; more preferably, from human.

In another preferred embodiment, the gene include wild-type gene and mutant-type gene.

In another preferred embodiment, the mutant-type includes mutant form that the function of the encoded protein is not changed after mutation (i.e., the function is identical or substantially identical to that of the wild-type encoded protein).

In another preferred embodiment, the polypeptide encoded by the mutant-type gene is the same or substantially the same as the polypeptide encoded by the wild-type gene.

In another preferred embodiment, the mutant-type gene comprises a polynucleotide having a homology of ≥80% (preferably ≥90%, ≥91%, ≥92%, ≥93% or ≥94%, more preferably ≥95%, ≥96% or ≥97%, even more preferably 98% or 99%) with the sequence of wild-type gene.

In another preferred embodiment, the mutant-type gene includes polynucleotide that is truncated or added 1-60 (preferably 1-30, more preferably 1-10) nucleotides at the 5′ end and/or 3′ end of the wild-type gene.

In another preferred embodiment, the gene includes a cDNA sequence, a genomic sequence, or a combination thereof.

In another preferred embodiment, the protein includes active fragment or derivative thereof.

In another preferred embodiment, the said active fragment or derivative has a homology of at least 90%, 91%, 92%, 93% or 94% with the said gene or its RNA or its encoded protein, preferably 95%, 96% or 97%, more preferably 98%, 99%.

In another preferred embodiment, the said active fragment or derivative has at least 80%, 81%, 82%, 83%, 84%, 85%, 86%, 87%, 88%, 89%, 90/9, 91%, 92%, 93%, 94%, 95%, 96%, 97%, 98%, 99% or 100% activity.

In another preferred embodiment, the amino acid sequence of the protein selects from:
(i) the polypeptide comprising the amino acid sequence shown in SEQ ID NO: 1, 3, 5, 7, 9, 11, 13, 15, 17, 19, 21, 23, 25, 27, 29, 31, 33, 35, 37, 39, 41, 43, 45, 47 or 49;
(ii) the polypeptide which is derived from (i) having one or several (such as 1-10) amino acid residues substitution, deletion or addition in the sequence shown in SEQ ID NO: 1, 3, 5, 7, 9, 11, 13, 15, 17, 19, 21, 23, 25, 27, 29, 31, 33, 35, 37, 39, 41, 43, 45, 47 or 49, and possessing the function like the said protein; or
(iii) the polypeptide having the amino acid sequence with ≥90%, ≥91%, ≥92%, ≥93% or ≥94% (preferably ≥95%, ≥96% or ≥97%, more preferably ≥98% or 99%) homology to the amino acid sequence shown in SEQ ID NO: 1, 3, 5, 7, 9, 11, 13, 15, 17, 19, 21, 23, 25, 27, 29, 31, 33, 35, 37, 39, 41, 43, 45, 47 or 49, and possessing the function like the said protein.

In another preferred embodiment, the nucleotide sequence of the said gene selects from:
(a) a polynucleotide encoding a polypeptide having the amino acid sequence shown above;
(b) a polynucleotide having the sequences shown in SEQ ID NO: 2, 4, 6, 8, 10, 12, 14, 16, 18, 20, 22, 24, 26, 28, 30, 32, 34, 36, 38, 40, 42, 44, 46, 48 or 50;
(c) a polynucleotide having the nucleotide sequence with ≥90%, ≥91%, ≥92%, ≥93% or ≥94% (preferably ≥95%, ≥96% or ≥97%, more preferably ≥98% or 99%) homology to the nucleotide sequence shown in SEQ ID NO: 2, 4, 6, 8, 10, 12, 14, 16, 18, 20, 22, 24, 26, 28, 30, 32, 34, 36, 38, 40, 42, 44, 46, 48 or 50;
(d) a polynucleotide having 1-60 (preferably 1-30, more preferably 1-10) nucleotide truncated or added at the 5′ end and/or 3′ end of the polynucleotide shown in SEQ ID NO: 2, 4, 6, 8, 10, 12, 14, 16, 18, 20, 22, 24, 26, 28, 30, 32, 34, 36, 38, 40, 42, 44, 46, 48, or 50;
(e) a polynucleotide complementary to the polynucleotide described in any one of (a)-(d).

In another preferred embodiment, the protein has the amino acid sequence mentioned above.

In another preferred embodiment, the encoding nucleic acid of the protein has the nucleotide sequence mentioned above.

In another preferred embodiment, the region targeted by the inhibitor (such as a gene editing protein) is the position 121931-121963 (RCOR1), the position 2364-2393 (RCOR2), the position 53556-53585 (RCOR3), the position 59338-59367 (Sin3a), the position 12378-12406 (Sin3b), the position 24648-24678 (HDAC1), or the position 60126-60154 (KDM1A).

In another preferred embodiment, the inhibitor or agonist inhibits or enhances the activity and/or expression of the gene or its RNA or its encoded protein.

In another preferred embodiment, the concentration of the inhibitor or agonist (titer of the virus) is >1×10¹².

In another preferred embodiment, the inhibition rate or enhancement rate of the inhibitor or agonist on the activity and/or expression of the gene or its RNA or its encoded protein is greater than 90%, preferably, 90%-95%.

In another preferred embodiment, the inhibitor or agonist targets astrocytes in brain tissue.

In another preferred embodiment, the inhibitor or agonist targets MG cells in the retina.

In another preferred embodiment, the gRNA guides the gene editing protein to specifically bind to the mRNA of the gene.

In another preferred embodiment, the composition includes a pharmaceutical composition.

In another preferred embodiment, the composition further includes other drugs for preventing and/or treating diseases associated with neuronal function loss or death.

In another preferred embodiment, the composition further includes other drugs for the treatment of neurological diseases associated with neuronal death.

In another preferred embodiment, the composition further includes other drugs for the prevention and/or treatment of retinal diseases.

In another preferred embodiment, the expression vector of the gene editing protein includes a vector targeting glial cells.

In another preferred embodiment, the expression vector of the gene editing protein includes a vector targeting astrocytes in brain tissue.

In another preferred embodiment, the expression vector of the gene editing protein includes a vector targeting MG cells in retina.

In another preferred embodiment, the vector includes AAV2 or AAV9.

In another preferred embodiment, the encoding gene of the gene editing protein is located in the same expression vector (such as an AAV vector) with the gRNA.

In another preferred embodiment, the expression vector of the gene editing protein and the expression vector of gRNA are the same expression vector (such as AAV vector).

In another preferred embodiment, the expression vector further includes a glial cell-specific promoter (e.g., GFAP promoter) for driving the expression of the gene editing protein.

In another preferred embodiment, the formulation of the composition selects from: lyophilized formulations, liquid formulations, or combination thereof.

In another preferred embodiment, the formulation of the composition is liquid formulation.

In another preferred embodiment, the formulation of the composition is injectable formulation.

In another preferred embodiment, other drugs for preventing and/or treating diseases associated with neuronal function loss or death select from: dopamine prodrugs, non-ergot dopamine receptor agonists, monoamine oxidase B inhibitors, or the combinations.

In another preferred embodiment, the composition is cellular preparation.

In another preferred embodiment, the expression vector of the gene editing protein and the expression vector of the gRNA are either the same vector or different vectors.

In another preferred embodiment, the weight ratio of component (a) to component (b) is 100:1-0.01:1, preferably, 10:1-0.1:1, more preferably, 2:1-0.5:1.

In another preferred embodiment, the content of component (a) in the composition is 0.001%-99% A, preferably 0.1%-90%, more preferably 1%-70%.

In another preferred embodiment, the content of component (b) in the composition is 0.001%-99%, preferably, 0.1%-90%, more preferably, 1%-70%.

In another preferred embodiment, the content of component (c) in the composition is %-99%, preferably 10%-90%, more preferably 30%-70%.

In another preferred embodiment, the component (a) and component (b) and optional component (c) constitute 0.01-99.99 wt % of the total weight of the composition, preferably 0.1-90 wt %, more preferably 1-80 wt %.

In another embodiment, this disclosure provides a medicine kit comprising:
(a1) the first container, and the gene editing protein or its expression vector or a medicament including the gene editing protein or its expression vector located in the first container, the gene editing protein selects from: Cas13d, Cas13e, Cas13a, Cas13b, Cas13c, Cas13f, RNA-targeted gene editing proteins, or combinations thereof;
(b1) a second container, and gRNA or its expression vector or a medicament containing gRNA or its expression vector located in the second container, the gRNA guides the gene editing protein specifically binding to the DNA or RNA of the gene.

In another preferred embodiment, the medicine kit further comprises:
(c1) a third container and other medicament for preventing and/or treating the disease associated with neuronal function loss or death, and/or retinal disease, and/or neurological diseases associated with neuronal death, the other medicament located in said third container.

In another preferred embodiment, the first container, the second container, and the third container are the same or different containers.

In another preferred embodiment, the medicament in the first container is a single-ingredient formulation containing a gene editing protein or its expression vector.

In another preferred embodiment, the medicament in the second container is a single-ingredient formulation containing gRNA or its expression vector.

In another preferred embodiment, the medicament in the third container is a single-ingredient formulation containing other medicines intended for the treatment of neurological diseases related to neuronal death.

In another preferred embodiment, the formulation of the medicament selects from: lyophilized preparations, liquid preparations, or combinations thereof.

In another preferred embodiment, the formulation of the medicament is an oral formulation or an injectable formulation. In another preferred embodiment, the medicine kit further contains instructions.

Specifically, this application also involves the following solutions:

Item 1. Methods for transdifferentiating non-neuronal cells of mammalian into neurons, which comprise:
provide a negative regulator that can reduce the expression of negative regulatory gene, wherein the negative regulatory gene includes RCOR1, RCOR2, RCOR3, Sin3a, Sin3b, HDAC1, HDAC2, KDM1A, PHF21A, BAF53a, G9a, USP14, HuR, BrG1, EZH2, CDYL, or HMG20B, and/or
provide a positive regulator that can increase the expression of positive regulatory gene, wherein the positive regulatory gene includes DPYSL2, BAF45b, SCF, HuB, HuC, HuD, CYP1B1, or BTRC,
wherein, an effective amount of the negative regulator or the positive regulator is brought to contact with the non-neuronal cells to induce the trans-differentiation of non-neuronal animal cells into neurons.

In a specific embodiment, the method is performed in vitro, ex vivo or in vivo.

In a specific embodiment, the protein sequences associated with the negative regulatory genes are as follows: The amino acid sequence of RCOR1 is shown in SEQ ID NO: 1. The amino acid sequence of RCOR2 is shown in SEQ ID NO:3. The amino acid sequence of RCOR3 is shown in SEQ ID NO:5. The amino acid sequence of Sin3a is shown in SEQ ID NO:7. The amino acid sequence of Sin3b is shown in SEQ ID NO: 9. The amino acid sequence of HDAC1 is shown in SEQ ID NO:11. The amino acid sequence of HDAC2 is shown in SEQ ID NO:13. The amino acid sequence of KDM1A is shown in SEQ ID NO:15. The amino acid sequence of PHF21A is shown in SEQ ID NO:17. The amino acid sequence of BAF53a is shown in SEQ ID NO:19. The amino acid sequence of G9a is shown in SEQ ID NO:21. The amino acid sequence of USP14 is shown in SEQ ID NO:23. The amino acid sequence of HuR is shown in SEQ ID NO:25. The amino acid sequence of BRG1 is shown in SEQ ID NO:27. The amino acid sequence of EZH2 is shown in SEQ ID NO: 29. The amino acid sequence of CDYL is shown in SEQ ID NO:31. The amino acid sequence of HMG20B is shown in SEQ ID NO:33.

In a specific embodiment, the protein sequences associated with the positive regulatory genes are as follows: The amino acid sequence of DPYSL2 is shown in SEQ ID NO: 35. The amino acid sequence of BAF45b is shown in SEQ ID NO:37. The amino acid sequence of SCF is shown in SEQ ID NO: 39. The amino acid sequence of HuB is shown in SEQ ID NO: 41. The amino acid sequence of HuC is shown in SEQ ID NO: 43. The amino acid sequence of HuD is shown in SEQ ID NO: 45. The amino acid sequence of CYP1B1 is shown in SEQ ID NO: 47. The amino acid sequence of BTRC is shown in SEQ ID NO: 49.

Item 2. The method according to Item 1, wherein, the non-neuronal cells are derived from human

Item 3. The method according to Item 1 or Item 2, wherein, the non-neuronal cells are stem cells, progenitor cells, or terminally differentiated cells; preferably, are glial cells; more preferably, the glial cells are astrocytes, oligodendrocytes, microglia, NG2 cells, Müller glial cells, glioma cells, or spiral ganglion glial cells; even more preferably, the glial cells are astrocytes or Müller glial cells.

Item 4. The method according to item 3, wherein, the non-neuronal cells are from brain, preferably, the non-neuronal cells are from cerebrum, midbrain, cerebellum, brainstem, or spinal cord; more preferably, from the striatum or substantia nigra in brain.

Item 5. The method according to item 4, wherein, the non-neuronal cells are astrocytes from the brain, preferably astrocytes are from the striatum or the substantia nigra.

Item 6. The method according to Item 4, wherein, the non-neuronal cells are from the eye, preferably, the non-neuronal cells are Müller glial cells from the eye.

Item 7. The method according to any one of Items 1 to 6, wherein, the neurons are dopaminergic neurons, retinal ganglion cells, photoreceptor cells, 5-HT neurons, NE neurons, ChAT neurons, motor neurons, GABA neurons, glutamatergic neurons, spinal cord neurons, spinal motor neurons, spinal sensory neurons, bipolar cells, amacrine cells, cochlear nerve cells, pyramidal neurons, interneurons, medium spiny neurons, Purkinje cells, granule cells, olfactory receptor neurons, or periglomerular cells, or combinations thereof.

Item 8. The method according to any one of Items 1 to 7, wherein, the negative regulatory gene is RCOR1, RCOR2, RCOR3, Sin3a, Sin3b, KDM1A, BAF53a, G9a, HuR, BrG1, or EZH2, and the non-neuronal animal cells are from brain or eyes.

Item 9. The method according to Item 8, wherein, the negative regulatory gene is RCOR1, RCOR2, RCOR3 or G9a, and the non-neuronal animal cells are from the brain; preferably, the non-neuronal cells are astrocytes from brain; more preferably, the non-neuronal cells are astrocytes from the striatum or substantia nigra.

Item 10. The method according to item 8, wherein, the negative regulatory gene is Sin3a, Sin3b, KDM1A, BAF53a, G9a, HuR, BrG1, CDYL, or EZH2, and the non-neuronal cells are from eyes; preferably, the non-neuronal cells are Müller glial cells from eyes.

Item 11. The method according to item 10, wherein, the negative regulatory gene is HuR, the neuron is retinal ganglion cell.

Item 12. The method according to Item 10, wherein the negative regulatory gene is Sin3a, KDM1A, BAF53a, G9a, HuR, BrG1, CDYL, or EZH2, the neuron is photoreceptor cell.

Item 13. The method according to any one of Items 1 to 7, wherein, the positive regulatory gene is DPYSL2, BAF45b, SCF, HuB, HuC, HuD, CYP1B1, or BTRC, and the non-neuronal cells are from brain; preferably, the non-neuronal cells are from the striatum, substantia nigra, ventral tegmental area of the midbrain, spinal cord, hypothalamus, dorsal midbrain, cerebral cortex, hippocampus, or cerebellum: more preferably, from the striatum.

Item 14. The method according to Item 13, wherein, the positive regulatory gene is SCF or HuB.

Item 1S. The method according to Item 13, wherein, the positive regulatory gene is HuB or BTRC, and an effective amount of the positive regulator contact with the non-neuronal cell to induce non-neuronal cell transdifferentiating into dopaminergic neurons.

Item 16. The method according to any one of Items 13 to 15, wherein, the non-neuronal cells are astrocytes: more preferably, the non-neuronal cells are astrocytes from the striatum or the substantia nigra.

Item 17. The method according to any one of Items 1 to 7, wherein, the positive regulatory gene is DPYSL2. BAF45b, SCF, HuC, HuD, or CYP1B1, and the non-neuronal cells are from eyes: preferably, the non-neuronal cells are Müller glial cells from eyes.

Item 18. The method according to Item 17, wherein, the positive regulatory gene is SCF, HuD, or CYP1B1, and the neuron is retinal ganglion cell.

Item 19. The method according to Item 17, wherein, the positive regulatory gene is DPYSL2 or BAF45b, and the neuron is photoreceptor cell.

Item 20. The method according to any one of Items 1 to 19, wherein, reducing the expression of the negative regulatory gene refers to decreasing the gene level of the negative regulatory gene, or decreasing the mRNA level of the negative regulatory gene, or decreasing the expression level of the protein encoded by negative regulatory gene;
wherein, increasing the expression of positive regulatory gene refers to increasing the gene level of positive regulatory gene, or increasing the mRNA level of positive regulatory gene, or increasing the expression level of protein encoded by positive regulatory gene.

Item 21. The method according to item 20, wherein, the negative regulator selects from gene editing tools or epigenetic regulatory tools for reducing the expression of negative regulatory gene, inhibitors of negative regulatory gene, activity inhibitors of negative regulator gene, or degradation activators of the protein encoded by a negative regulatory gene.

Item 22. The method according to Item 21, wherein, the inhibitor is: an inhibitory antibody of the negative regulatory gene; or a small molecule inhibitor of the negative regulatory gene; or an inhibitory mRNA, microRNA, siRNA, shRNA, antisense oligonucleotide, binding protein or protein domain, polypeptide, nucleic acid aptamer, or PROTAC of the negative regulatory gene; or inhibitory binding protein or ligand of the negative regulatory gene.

Item 23. The method according to Item 21, wherein, the negative regulator contains the gRNA as shown in any one of SEQ ID NO: 51-67 and gene editing protein.

Item 24. The method according to item 20, wherein, the positive regulator selects from epigenetic regulatory tools capable of increasing expression of positive regulatory gene, expression activators of positive regulatory gene, degradation inhibitors of proteins encoded by positive regulatory gene, mRNA stabilizers of positive regulatory gene, or exogenous positive regulatory gene or functional fragment of positive regulatory gene.

Item 25. The method according to Item 24, wherein, the activator is: an agonistic antibody of positive regulatory gene; or a small molecule agonist of positive regulatory gene; or an agonistic binding protein or ligand of positive regulatory gene; or an inhibitor of competitive genes of positive regulatory gene.

Item 26. The method according to item 24, wherein, the positive regulator contains a nucleic acid sequence shown in SEQ ID NO: 36, 38, 40, 42, 44, 46, 48, or 50, or contains a functional fragment of the nucleic acid sequence shown in SEQ ID NO: 36, 38, 40, 42, 44, 46, 48, or 50.

Item 27. The gene editing tool according to item 21, wherein, the gene editing tool includes a gene editing system or its expression vector, the gene editing system selects from: CRISPR system (including CRISPR/Cas system), ZFN system, TALEN system, or combination thereof.

Item 28. The method according to item 27, wherein, a CRISPR system is used to reduce the expression or activity of negative regulatory gene; preferably, the CRISPR system contains a nucleic acid encoding a Cas enzyme or a nucleic acid encoding the functional domain of Cas enzyme, as well as gRNA targeting the cellular trans-differentiation factor, more preferably, the Cas enzyme is Cas13; more preferably, the Cas enzyme is Cas13d, Cas13X, Cas13a, Cas13b, Cas13c, or Cas13Y; even more preferably, the Cas enzyme is CasRx.

Item 29. The method according to item 20, wherein, the negative regulator or positive regulator is carried by a carrier; preferably, the carrier is viral vector, lipid nanoparticle (LNP), lipidosome, cationic polymer (such as PEI), nanoparticle, exosome, or virus-like particle; more preferably, the carrier is AAV vector or lipid nanoparticle.

Item 30. The method according to any one of Items 1 to 29, wherein, the effective amount of the negative regulator or the positive regulator contacts with the non-neuronal cells in vitro to induce non-neuronal cells transdifferentiating into neurons in vitro; or
the effective amount of the negative regulator or the positive regulator contacts the non-neuronal cells in vivo to induce the non-neuronal cells transdifferentiating into neurons in vivo.

Item 31. Use of the negative regulator or positive regulator according to any one of Items 1 to 30 for preparing a medicament to prevent or treat diseases associated with neuronal injury or neuronal death.

Item 32. Use of the negative regulator or positive regulator according to any one of Items 1 to 30 for preventing or treating diseases associated with neuronal injury or neuronal death.

Item 33. The use according to Item 31 or 32, wherein, the medicament is formulated as an agent for in vivo administration to the nervous system, visual system and auditory system, for example, for in vivo administration to the striatum, substantia nigra, midbrain ventral tegmental area, spinal cord, hypothalamus, dorsal midbrain, cerebral cortex, hippocampus, cerebellum, subretina, vitreous cavity, inner ear cochlea and vestibulum, preferably administration to striatum, substantia nigra, subretina and vitreous cavity.

Item 34. The use according to Item 31 or 32, wherein, the disease associated with neuronal injury or neuronal death selects from Parkinson's disease, visual system diseases related to function loss or death of RGC or photoreceptor cell, Alzheimer's disease, brain injury, Huntington's disease, epilepsy, depression, sleep disorders, cerebral ischemia, motor neuron disease, amyotrophic lateral sclerosis, spinal muscular atrophy, ataxia, PloyQ disease, schizophrenia, addiction, Pick's disease, blindness, and deafness; preferably, selects from Parkinson's disease and visual system diseases related to function loss or death of RGC or photoreceptor cell.

Item 35. The use according to Item 34, wherein, the visual system disease related to function loss or death of RGC or photoreceptor cell preferably selects from: visual impairment due to the death of RGC cell or photoreceptor cell, glaucoma, age-related RGC pathology, optic nerve injury, age-related macular degeneration (AMD), diabetes-related retinopathy, retinal ischemia or hemorrhage, Leber's hereditary optic neuropathy, or a combination thereof; the visual system disease related to the function loss or death of photoreceptor cell preferably selects from: photoreceptor cell degeneration or death caused by injury or degenerative disease, macular degeneration, retinitis pigmentosa, diabetes-related blindness, nyctalopia, color blindness, hereditary blindness, congenital amaurosis, or a combination thereof.

Item 36. The use according to Item 31 or 32, wherein, the neurons are dopaminergic neurons, 5-HT neurons, NE neurons, ChAT neurons, GABA neurons, glutamatergic neurons, motor neurons, photoreceptor cells (such as rods and cones), retinal ganglion cells (RGC), cochlear nerve cells (such as cochlear spiral ganglion cells and vestibular neurons), or medium spiny neurons (MSN) or combinations thereof, preferably are dopaminergic neurons, retinal ganglion cells or photoreceptor cells.

Item 37. A method for preventing or treating diseases associated with neuronal injury or neuronal death, comprise administering an effective amount of the negative regulator or positive regulator according to any one of items 1 to 30 to a subject in need.

Item 38. The method according to Item 37, wherein, the disease related to neuronal injury or neuronal death selects from Parkinson's disease, visual system diseases related to function loss or death of RGC or photoreceptor cell, Alzheimer's disease, brain injury, Huntington's disease, epilepsy, depression, sleep disorders, cerebral ischemia, motor neuron disease, amyotrophic lateral sclerosis, spinal muscular atrophy, ataxia, PloyQ disease, schizophrenia, addiction, Pick's disease, blindness, deafness; preferably selected from Parkinson's disease and visual system diseases related to function loss or death of RGC or photoreceptor cell.

Item 39. The method according to Item 38, wherein, the visual system disease related to function loss or death of RGC preferably selects from: vision impairment due to the death of RGC cell, glaucoma, age-related RGC pathology, optic nerve injury, age-related macular degeneration (AMD), diabetes-related retinopathy, retinal ischemia or hemorrhage, Leber's hereditary optic neuropathy, or combinations thereof; the visual system disease related to the function loss or death of photoreceptor cell preferably selects from: photoreceptor cell degeneration or death caused by injury or degenerative disease, macular degeneration, retinitis pigmentosa, diabetes-related blindness, nyctalopia, color blindness, hereditary blindness, congenital amaurosis, or combinations thereof.

Item 40. The method according to Item 37, wherein, the neurons are dopaminergic neurons, 5-HT neurons, NE neurons, ChAT neurons, GABA neurons, glutamatergic neurons, motor neurons, photoreceptor cells (such as rods and cones), retinal ganglion cells (RGC), cochlear nerve cells (such as cochlear spiral ganglion cells and vestibular neurons), or medium spiny neurons (MSN) or combinations thereof, preferably, are dopaminergic neurons, retinal ganglion cells or photoreceptor cells.

The technical solutions involved in this application also include:
the use of the negative regulator or positive regulator associated with KDM1A or EZH2 according to any one of Items 1 to 30, for preparing a medicament to prevent or treat diseases associated with optic neuronal injury or optic neuronal death.

In some embodiments, the disease associated with optic neuronal injury or optic neuronal death can be retinitis pigmentosa (RP).

It should be understood that within the scope of this disclosure, the various technical features mentioned above and those specifically described hereinafter (such as examples) can be combined with each other to constitute new or preferred technical solutions. Due to limited space, these will not be listed exhaustively here.

### DESCRIPTION OF FIGURES

FIG. 1: Results of specifically knocking down each target gene by CasRx in vitro. In the N2A cell line in vitro, expressing CasRx and gRNA targeting RCOR1, Sin3a, HDAC2, PHF21A, BAF53a, KDM1A, G9a, USP14, HuR, BrG1, EZH2, CDYL, or HMG20B, can achieve high-efficiency knockdown of RCOR1, Sin3a, HDAC2, KDM1A, PHF21A, BAPS3a, G9a, USP14, HuR, BrG1, EZH2, CDYL, or HMG20B.

FIG. 2: Schematic diagram of transdifferentiating mouse astrocytes into neurons by knocking down or overexpressing different target genes. FIG. 2A: Fluorescent protein mCherry is driven to express by the astrocyte-specific promoter GFAP in vector 1 (AAV-GFAP-mCherry), the RNA-editing protein CasRx is driven to express by the astrocyte-specific promoter GFAP in vector 2 (AAV-GFAP-CasRx), vector 3 (AAV-GFAP-CasRx-sg) encodes CasRx and gRNA targeting RCOR1, Sin3a, HDAC2, KDM1A, PHF21A, BAF53a, G9a, USP14, HuR, BrG1, EZH2, CDYL, or HMG20B; FIG. 2B: vector 1 shows a schematic diagram of a GFAP-driven mCherry vector, vector 4 shows a schematic diagram of a vector expressing target gene driven by GFAP, the target gene is DPYSL2, BAF45b, SCF, HuB, HuC, HuD, CYP1B1, or BTRC, respectively.

FIG. 3: The conversion of astrocytes into neurons in the mouse striatum after knocking down different negative regulatory genes. FIG. 3A shows that AAV-GFAP-mCherry can specifically label astrocytes and astrocytes don't be transdifferentiated into neurons after injection of control group AAV (AAV-GFAP-mCherry+AAV-GFAP-After CasRx) into the mouse striatum. The yellow arrows (the arrows shown in FIG. 3A are yellow) point to the astrocytes labeled by GFAP-mCherry, which are not co-labeled with NeuN. NeuN is a neuron-specific marker. FIG. 3B to FIG. 3M present the results of transdifferentiating astrocytes into neurons by using CasRx to knock down RCOR1, HDAC2, PHF21A, BAF53a, G9a, USP14, HuR, BrG1, EZH2, CDYL, HMG20B, or KDM1A in the mouse striatum, respectively. The red signal represents the cells labeled by AAV-GFAP-mCherry (the leftmost column shown from FIG. 3A to FIG. 3M show the results labeled by mCherry, the bright parts in the figures are red). NeuN is a neuron-specific marker, and the white arrows point to cells that are co-labeled with the red mCherry signal and NeuN (the arrows shown from FIG. 3B to FIG. 3M are white arrows). Scale bar is 50 microns.

FIG. 4: Statistical diagram of transdifferentiating astrocytes into neurons after knocking down different negative regulatory genes.

FIG. 5: The conversion of astrocytes into neurons in the mouse striatum after overexpression of different positive regulatory genes. FIG. 5A shows the result of specifically labeled astrocytes in the striatum of the mouse brain after injecting control group AAV (AAV-GFAP-mCherry). The yellow arrows (the arrows shown in FIG. 5A are yellow) point to the astrocytes labeled by GFAP-mCherry, which are not co-labeled with NeuN, NeuN is a neuron-specific marker. FIG. 5B to FIG. 51 present the results of injecting the test group AAV (AAV-GFAP-mCherry+AAV-GFAP-positive regulatory gene) into the striatum of the mouse brain, the positive regulatory gene is DPYSL2, BAF45b, SCF, HuB, HuC, HuD, CYP1B1, or BTRC. The white arrows point to cells co-labeled with the red mCherry signal and NeuN (the leftmost column shown in FIG. 5A to FIG. 5I shows the results labeled by mCherry, the bright parts in the figures are red, the arrows shown in FIG. 5B to FIG. 5I are white arrows). NeuN is a neuron-specific marker. Scale bar is 50 microns.

FIG. 6: Statistical diagram of transdifferentiating astrocytes into neurons after up-regulation of different positive regulatory genes.

FIG. 7: The conversion of astrocytes into dopaminergic neurons after overexpression of BTRC or HuB in mouse striatum. FIG. 7A shows the result of injection of control group AAV (AAV-GFAP-mCherry) into the striatum of mice. The yellow arrows (the arrows shown in FIG. 7A are yellow) point to the labeled mCherry-positive cells that do not co-label with TH and NeuN, TH is a dopaminergic neuron-specific protein marker, NeuN is a neuron-specific marker. FIG. 7B and FIG. 7C present the results of injecting the test group AAV (AAV-GFAP-mCherry+AAV-GFAP-positive regulatory genes) into the striatum of the mouse brain respectively, wherein the positive regulatory gene is HuB or BTRC. The white arrows (the arrows shown in FIGS. 7B and C are white) point to cells co-labeled with the red mCherry signal, NeuN, and TH. Scale bar is 50 microns. The bright parts in the mCherry column are red, the bright parts in the TH column are green, the bright parts in the NeuN column are white, and the bright parts in DAPI column are blue.

FIG. 8: Schematic diagram of transdifferentiating Müller glial cells into retinal ganglion cells or photoreceptor cells by overexpressing or knocking down the target gene in the retina. In FIG. 8A, vector 1 is a schematic diagram of the plasmid AAV-GFAP-EGFP-2A-Cre, the expression of Cre is driven by the astrocyte-specific promoter GFAP, vector 2 is a schematic diagram of the plasmid AAV-GFAP-CasRx, the expression of the RNA-editing protein CasRx is driven by astrocyte-specific promoter GFAP, vector 3 is a schematic diagram of the plasmid of AAV-GFAP-CasRx-gRNA, encoding CasRx and the gRNA targeted negative regulatory genes (Sin3a, HDAC2, KDM1A, BAF53a, G9a, HuR, BrG1, EZH2, CDYL). FIG. 8B is a schematic of transdifferentiating Müller glial cells into retinal ganglion cells or photoreceptor cells by overexpression various positive regulatory gene in the retina, mCherry is driven by GFAP in vector 1, vector 4 shows the target gene is driven to express by GFAP, the target gene is DPYSL2, BAF45b, SCF, CYP1B1, or BTRC.

FIG. 9: The conversion of Müller glial cells into photoreceptor cells by knocking down negative regulatory genes in the mouse eye. FIG. 9A shows the result of injection of control group AAV (AAV-GFAP-EGFP-P2A-Cre) into the retina of Ai9 mice to specifically label Müller glial cells, the yellow arrows (the arrows shown in FIG. 9A are yellow) indicate the Müller glial cells specifically labeled by GFAP-EGFP-P2A-Cre, the cell body of Müller glial cell are located in the INL layer, there are no tdTomato-labeled red cells found in the ONL layer. FIG. 9B to FIG. 9J respectively show the results of subretinal injection of different AAVs (AAV-GFAP-mCherry+AAV-GFAP-CasRx-gRNA) into the mice of the test group, wherein the gRNA targets Sin3a, HDAC2, BAF53a, G9a, HuR, BrG1 EZH2, CDYL, or KDM1A, the white arrows indicate tdTomato-positive cells in the ONL layer, yellow arrows indicate tdTomato-positive Müller glial cells in the INL layer, the leftmost columns in FIG. 9A to FIG. 9J show the results labeled by mCherry, the bright parts in the figures are red, the arrows shown in the INL, columns in FIG. 9A to FIG. 9J are yellow, and the arrows shown in the ONL columns are white.

FIG. 10: FIG. 10A and FIG. 10B respectively show the transformation of Müller glial cells into photoreceptor cells after subretinal injection of test group AAV (AAV-GFAP-EGFP-P2A-Cre+AAV-GFAP-positive regulatory gene) in mice, the positive regulatory genes is DPYSL2 or BAF45b, the white arrows indicate tdTomato-positive cells in the ONL layer, the yellow arrows indicate tdTomato-positive Müller glial cells in the INL layer. The scale bar is 50 microns. The leftmost columns in the figures shows the result labeled by mCherry. The bright parts in the left column are red. The arrows shown in the INL column in the figure are yellow, and the arrows shown in the ONL column are white.

FIG. 11: Statistical diagram of reprogramming Müller glial cells into photoreceptor cells.

FIG. 12: The conversion of Müller glial cells into rod cells in mouse eyes with Sin3a or KDM1A knockdown. FIG. 12A shows the results of injecting AAV-GFAP-EGFP-P2A-Cre+AAV-GFAP-CasRx into the retina of Ai9 mice, the white signal represents staining of the rods-specific protein marker rhodopsin, and the yellow arrows (the arrows shown in FIG. 12A are yellow) indicate the tdTomato-labeled Müller glial cells located in the INL layer, there are no tdTomato-positive cells in the ONL layer. FIG. 12B and FIG. 12C show the trans-differentiation results following subretinal injection of test group AAV targeting Sin3a and KDM1A (AAV-GFAP-mCherry-AAV-GFAP-CasRx-gRNA) in Ai9 mouse retinas, the white arrows indicate tdTomato-positive cells located in the ONL layer that co-label with rhodopsin, the scale bar is 50 microns, the leftmost columns from FIGS. 12A to 12C show the results of labeled by mCherry, the bright parts in the left columns are red, and the arrows shown in FIGS. 12B and C are white arrows.

FIG. 13: The conversion result of Müller glial cells into cones when knock down Sin3a or EZH2 in mouse eyes. FIG. 13A shows the results of injecting AAV-GFAP-EGFP-P2A-Cre into the retina of Ai9 mice, GFAP-EGFP-P2A-Cre can specifically label Müller glial cells. The cell bodies of Müller glial cells are located in the INL layer, no tdTomato-labeled red cells are found in the ONL layer, the green signal is the EGFP fluorescence expressed by GFAP-EGFP-P2A-Cre, the yellow arrows (the arrows shown in FIG. 13A are yellow) indicate the Müller glial cells co-labeled with the green signal and the red signal. FIG. 13B shows the results of injecting control group AAV (AAV-GFAP-EGFP-P2A-Cre+AAV-GFAP-CasRx) into the retina of Ai9 mice, the white signal is m-CAR that is the cone cell specific protein marker, the cyan arrows (the arrows shown in FIG. 13B are cyan) indicate to tdTomato-positive Müller glial cells, no tdTomato-positive cells are found in the ONL layer. FIG. 13C and FIG. 13D show the results of subretinal injection of test group AAV (AAV-GFAP-mCherry+AAV-GFAP-CasRx-gRNA) targeting Sin3a and EZH2 in mice, respectively, white arrows (arrows shown in FIG. 13C and FIG. 13D are white) indicate the tdTomato-positive cells co-labeled with mCAR in the ONL layer. Scale bar is 50 microns. Wherein, the bright parts in the mCherry column are red, the bright parts in the EGFP column are green, and the bright parts in the DAPI column are blue.

FIG. 14: The conversion of Müller glial cells into retinal ganglion cells after knocking down the negative regulatory genes in mouse eyes. FIG. 14A shows the results of injecting control group AAV (AAV-GFAP-EGFP-P2A-Cre) into the retina of Ai9 mice, there is almost no labeled optic nerve. FIGS. 14B to 141 respectively show the results of subretinal injection of test group AAV (AAV-GFAP-mCherry+AAV-GFAP-CasRx-gRNA) in Ai9 mice to inhibit different negative regulatory genes, wherein, the gRNA respectively targets HDAC2, BAF53a, G9a, HuR, BrG1, EZH2, CDYL, or KDM1A.

FIG. 15: FIGS. 15A to 15G show the transformation results of Müller glial cells into retinal ganglion cells by overexpressing different positive regulatory genes in mouse eyes. The positive regulatory gene is DPYSL2, BAF45b, SCF, HuB, HuD, CYP1B1, or BTRC, and the scale bar is 150 microns.

FIG. 16: Statistical diagram of reprogramming Müller glial cells into retinal ganglion cells.

FIG. 17: Results of reprogramming Müller glial cells into photoreceptor cells in mouse model of retinitis pigmentosa. FIG. 17A is a schematic diagram of the degeneration and death of rod cells and cone cells as well as the timing of AAV injection in the retinitis pigmentosa disease model mouse (Pde6b-KO). A AV injection is performed after the complete death of photoreceptor cells in the retina of Pde6b-KO mice (P50), and retinal tissue is collected for analysis 50 days after injection. FIG. 17B and FIG. 17C show the results of immunofluorescence staining using rods-specific marker rhodopsin on retinal tissues collected from Pde6b-KO mice at 4 weeks and 6 weeks of age, respectively, no rod cells are observed at either 4 or 6 weeks. FIG. 17D presents the result of immunofluorescence staining performed on retinal tissue sections after the injection of AAV (GFAP-CasRx+CBH-Pde6b) in control group, there are no rhodopsin-positive cells in the ONL layer. FIG. 17E to FIG. 17F show the result of immunofluorescence staining performed on retinal tissue sections after the injection of AAV (GFAP-CasRx-gRNA+CBH-Pde6b) in treatment group, gRNA target and knock down the expression of EZH2 or KDM1 Å, the rhodopsin-positive cells reappeared in the ONL layer; wherein, the bright parts in the rhodopsin column are green, and the bright parts in the DAPI column are blue.

FIG. 17G is a statistical diagram of the number of photoreceptor cells in the retina of different treatment groups of Pde6b-KO mice, 4w and 6w show the number of photoreceptor cell in the retina of Pde6b-KO mice without AAV injection, the number of photoreceptor cells significantly increase in the group of EZH2 or KIMIA knockdown. FIG. 17H shows the thickness of the retinal ONL layer in different treatment groups of Pde6b-KO mice, 4w and 6w show the thickness of the retinal ONL layer of Pde6b-KO mice without AAV injection, the thickness significantly increases in the EZ2 or KDM1A knockdown group.

### DETAILED WAYS

After extensive and in-depth research, the inventors unexpectedly discovered for the first time that inhibiting the expression or activity of one or more genes or their RNAs or their encoded proteins selected from the group consisting of RCOR1, RCOR2, RCOR3, Sin3a, Sin3b, HDAC1, HDAC2, KDM1A, PHF21A, BAF53a, G9a, USP14, HuR, BRG1, EZH2, CDYL, and HMG20B in glial cells, and/or enhancing the expression or activity of one or more genes or their RNAs or their encoded proteins selected from the group consisting of DPYSL2, BAF45b, SCF, HuB, HuC, HuD, CYP1B1, and BTRC in glial cells, can effectively induce the differentiation of glial cells into neuronal cells, thereby treating neurological diseases associated with neuronal function loss or death. Upon these discoveries, the inventors complete the technical solution of this application.

Unless defined otherwise, the use of the singular forms “a”, “an” and “the” in this text also include plural objects. For example, reference to “an inhibitor” may include a plurality of inhibitors, and a reference to “the reagent” includes one or more reagents, as well as its equivalent known to those skilled in the art.

Unless defined otherwise, all technical and scientific terms used herein have the same meaning as commonly understood by the ordinary skilled in the art. Although the methods and reagents similar or equivalent to those described herein all can be used in the practice of the disclosed methods and compositions, exemplary methods and materials are now described.

Unless defined otherwise, the use of “and” herein also includes “and/or” and, similarly, “comprise” and “include” are interchangeable and are not intended to be limiting.

It should be further understood that those skilled in the art will understand that, in some specific instances, when the term “comprising” is used in the description of embodiments, the embodiments may alternatively be described as “consisting essentially of . . . ” or “consisting of”.

In this disclosure, retinal ganglion cell (RGC) degeneration is the primary cause of permanent blindness. The trans-differentiation of Müller glial cells (MG) into RGCs can contribute to vision restoration. The inventors found that MGs can be directly converted into RGC by reducing (e.g., knocking down using the RNA-targeted CRISPR system CasRx) the gene or its RNA or its encoded protein in mature mouse retinas. Furthermore, in a mouse model of retinal damage induced by NMDA (N-methyl-D-aspartate), RGCs converted from MGs achieves functional projections to central visual areas and improve the visual function. Therefore, reducing negative regulatory genes (e.g., through CasRx-mediated knockdown) would be a promising therapy for the treatment of retinal diseases caused by neurodegeneration.

This application uses CasRx which is the recently characterized as RNA-targeting CRISPR system to inhibit the gene or its RNA or its encoded protein.

As used herein, Müller glia (MG) are the main glial cells in retinal tissue, and retinal ganglion cells (RCC) are neuronal cells located in the innermost layer of the retina. The dendrites of RGCs primarily connect with bipolar cells, and the axons extend to the optic disc, which form the optic nerve.

Unless defined otherwise, the terms “differentiation,” “reprogramming” or “trans-differentiation” have the same meaning herein and may refer to the process of generating cells of a specific lineage (e.g., neuronal cells) from different types of non-neuronal cells (e.g., astrocytes) without intermediate differentiation.

As used herein, “a negative regulator or a positive regulator is brought to contact with the non-neuronal cell” refers to placing a negative regulator or a positive regulator (e.g., compound, nucleic acid, viral vector, etc.) in a position that allows to contact with the cells, thereby generating “contacted” cells. Contacting may be accomplished using any suitable method. For example, in one embodiment, contacting is achieved by adding negative regulator or positive regulator to the cell culture. Contact can also be accomplished by injecting the negative or positive regulator or a carrier containing the negative or positive regulator into a location in the body; or accomplished by delivering the negative or positive regulator or a carrier containing the negative or positive regulator to a location in the body.

In some embodiments, the effective amount of the negative regulator or the positive regulator contacts with the non-neuronal cell in vivo to induce trans-differentiation of the non-neuronal cells into neurons in vivo. The positive or negative regulator is formulated to a medicament for in vivo administration to the nervous system, visual system, and auditory system, for example, in vivo administrated to the striatum, substantia nigra, ventral tegmental area of the midbrain, spinal cord, hypothalamus, dorsal midbrain, cerebral cortex, hippocampus, cerebellum, subretina, vitreous cavity, inner ear cochlea or vestibulum, preferably administrated to striatum, substantia nigra, subretina and vitreous cavity.

In some embodiments, the effective amount of the negative regulator or the positive regulator contacts with the non-neuronal cell in vitro to induce trans-differentiation of the non-neuronal cells into neurons in vitro. In some specific embodiments, the neurons obtained through the in vitro contact can be formulated into an appropriate cell therapy agent, which is then administered to the individual in need via suitable administration methods, such as intravenous infusion, in situ injection, etc.

The term “ONL” refers to the outer granule cell layer, the term “INL” refers to the inner granule cell layer, and the term “GCL” refers to the retinal ganglion cell layer.

As used herein, the term “non-neuronal cell” may refer to any type of cell that is 3A not a neuron. In some embodiments, the non-neuronal cells are stem cells, progenitor cells, or terminally differentiated cells.

Exemplary non-neuronal cells are the cell lineages (e.g., hematopoietic lineage) other than the neuronal lineage. In some embodiments, the non-neuronal cells belong to the neuronal lineage but are not neurons, such as glial cells. In some embodiments, the non-neuronal cells are terminally differentiated cells but not neurons, such as but not limited to, glial cells, fibroblasts, embryonic fibroblasts, epithelial cells, melanocytes, keratinocytes, adipocytes, blood cells, marrow stromal cells, Langerhans cells, muscle cells, rectal cells, or chondrocytes. In some embodiments, the non-neuronal cells are derived from a non-neuronal cell line, such as but not limited to, glioblastoma cell line, Hela cell line, NT2 cell line, ARPE19 cell line, or N2A cell line. The term “cell lineage” or “lineage” refer to the developmental history of a tissue or organ from a fertilized embryo. The term “neuronal lineage” may refer to the developmental history from neural stem cells to mature neurons, including various stages along this process (called as neurogenesis), such as but not limited to, neural stem cells (neuroepithelial cells, radial glia), neural progenitor cells (e.g., interneuron precursors), neurons, astrocytes, oligodendrocytes, and microglia.

In some embodiments, the non-neuronal cells are stem cells, such as embryonic stem cells, neural stem cells, or induced pluripotent stem cells. In some embodiments, the non-neuronal cells are progenitor cells, such as neural grandmother cells or neural precursor cells (e.g., dopamine neural precursor cells).

In some specific embodiments, the non-neuronal cells are from the brain.

Preferably, the non-neuronal cells are from the cerebrum, midbrain, cerebellum, brainstem, or spinal cord; more preferably, they are from striatum or substantia nigra in brain. Preferably, the non-neuronal cells are astrocytes from brain, more preferably, astrocytes are from the striatum or substantia nigra. In some specific embodiments, the non-neuronal cells are from eyes, preferably, the non-neuronal cells are Müller glial cells from eyes.

The term “progenitor cell” refers to intermediate cells present before cells differentiate into terminally differentiated cells in adult tissues. The differentiation of progenitor cells is usually well-defined.

The term “terminal differentiated cells”, also known as terminal cells, refers to cells having specific type and specific functional, and no longer undergo differentiation, division and proliferation. For example: glial cells, somatic cells, fibroblasts, red blood cells, mature epidermal cells, muscle cells, etc.

The term “stem cells” should be understood as undifferentiated cells that have differentiation potential and proliferation capacity (in particular self-renewal capacity). According to differentiation potential, stem cells include subpopulations such as pluripotent stem cells (PSC), multipotent stem cells, unipotent stem cells, embryonic stem cells, etc. In some implementations, the stem cells can be embryonic stem cells, neural stem cells, or induced pluripotent stem cells.

As used herein, the term “pluripotent stem cells” (PSC) refers to stem cells that can be cultured in vitro and have the ability to differentiate into any cell lineage belonging to the three germ layers (ectoderm, mesoderm, endoderm). PSCs can be induced from oosperm, cloned embryos, germline stem cells, stem cells in tissues, somatic cells, etc. Examples of PSCs include embryonic stem cells (ESCs), induced pluripotent stem cells (iPSCs or ips), embryonic germ cells (EG cells), and the like.

As used herein, the term “induced pluripotent stem cells” (iPS or iPSC) can be generated directly from somatic cells through reprogramming. The somatic cells can be converted into PSCs by introducing the products of a specific set of pluripotency-related genes.

As used herein, the term “neural precursor cells” refers to cells that have neuronal development potential and are in a precursor state of neuronal development.

The term “neuron” may have its general meaning as understood by those skilled in the art. In some embodiments, a neuron may refer to an electrically excitable cell that can receive, process, and transmit information through electrical signals (e.g., membrane potential discharge) and chemical signals (e.g., synaptic transmission of neurotransmitters). As understood by those skilled in the art, the transduction of chemical signals between neurons (e.g., based on the release and recognition of neurotransmitters) can occur through specialized connections by synapses. In some embodiments, the neurons are dopaminergic neurons, retinal ganglion cells, photoreceptor cells, 5-HT neurons, NE neurons, ChAT neurons, motor neurons, GABA neurons, glutamate neurons, spinal cord neurons, spinal motor neurons, spinal sensory neurons, bipolar cells, amacrine cells, cochlear nerve cells, pyramidal neurons, interneurons, medium spiny neurons, Purkinje cells, granule cells, olfactory receptor neurons, or periglomerular cells, or combinations thereof.

As used herein, the terms “protein”, “peptide” and “polypeptide” have the same meaning and may refer to an amino acid polymer or a group having two or more interacting or combined amino acid polymers.

As used herein, the terms “nucleic acid” and “polynucleotide” have the same meaning and may refer to a nucleic acid molecule containing one or more nucleotides.

In this article, the terms “dopaminergic neuron” and “dopamine neuron” have the same meaning.

As used herein, the term “target gene” refers to a gene regulated by a “negative regulator” or a “positive regulator”. For example: RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B, DPYSL2, BAF45b, SCF, HuB, HuC, HuD, CYP1B1, or BTRC.

The term “photoreceptor cell” refers to a type of neuroepithelial cell in the retina that is responsible for visual light transmission, which include rods and cones. Certain conditions such as retinitis pigmentosa, macular degeneration, diabetes complications can lead to the death of photoreceptor cells.

The term “ganglion cells” are neurons located in the final segment of the retina.

Many oculopathies can lead to the death of retinal ganglion cells, such as ischemic retinopathy, glaucoma, etc. The death of retinal ganglion cells may also lead to permanent blindness.

As used herein, Müller glia (MG) are the main glial cells in retinal tissue, and retinal ganglion cells (RGC) are neuronal cells located in the innermost layer of the retina. The dendrites of RGCs primarily connect with bipolar cells, and the axons extend to the optic disc, which form the optic nerve.

The term “carrier” refers to a tool that can transport exogenous nucleic acids into cells, which can be nucleic acids, proteins, etc. When a vector is present in the appropriate environment, the vector is capable of guiding the expression of one or more proteins encoded by one or more genes carried by the vector, or capable of guiding the expression of microRNA encoded by a polynucleotide carried by the vector. In some embodiments, vectors can be used to transport the polynucleotides provided herein. In this context, the vector can be various forms. In some specific embodiments, the vector can be viral vector, plasmid vector, minicircle vector, linear DNA vector, doggybone vector, liposome, nanoparticle, exosome, extracellular vesicle, cationic polymer (such as PEI) or virus-like particle, etc.

The term “viral vector” refers to a nucleic acid of viral origin capable of transporting another nucleic acid into a cell. When a viral vector present in an appropriate environment, the viral vector is capable of guiding the expression of one or more proteins encoded by one or more genes carried by the vector, or capable of guiding the expression of microRNA encoded by a polynucleotide carried by the vector. Examples of viral vectors include, but are not limited to, retroviral vectors, adenoviral vectors, lentiviral vectors, poxviral vectors, herpesviral vectors, and adeno-associated viral vectors.

In some embodiments, nanoparticle carriers may include polymer-based nanoparticles, amino lipid-based nanoparticles, and metal nanoparticles.

The vectors provided herein can be used to deliver the polynucleotide compositions provided in this context. In some cases, a single vector is used to deliver at least about 2, 3, 4, or up to 5 polynucleotides. In some cases, a single vector is used to deliver at least about 2, 3, 4, or up to 5 different polynucleotides. In some cases, a single vector is used to deliver at least about 2, 3, 4, or up to 5 identical polynucleotides.

In some embodiments, the vector can deliver DNA (e.g., double-stranded DNA or single-stranded DNA) as well as RNA. In some cases, RNA can include base modifications. Vectors may include recombinant vectors. The vector may be a vector modified from a naturally occurring vector. The vector may include at least a portion of a non-naturally occurring carrier. Any carrier can be utilized.

As used herein, “astrocytes” may refer to the characteristic star-shaped glial cells in brain and spinal cord. It will be clear to those skilled in the art that astrocytes can be characterized by their stellate morphology and expression of markers such as glial fibrillary acidic protein (GFAP), aldehyde dehydrogenase 1 family member L1 (ALDH1L1), excitatory amino acid transporter 1/glutamate aspartate transporter (EAAT1/GLAST), glutamine synthetase, S1000 or excitatory amino acid transporter 1/glutamate transporter 1 (EAA2/GLT-1). They participate in forming the blood-brain barrier with endothelial cells, mediate neurotransmitter uptake and release, regulate ion concentrations in the extracellular space, respond to neuronal injury, participate in neural repair, and provide metabolic support to surrounding neurons. In some embodiments of the invention, astrocytes may refer to non-neuronal cells in the nervous system that express glial fibrillary acidic protein (GFAP), aldehyde dehydrogenase 1 family member L1 (ALDH1L1), or both. In some embodiments, astrocytes may refer to non-neuronal cells in the nervous system that express transgenes (e.g., red fluorescent protein (RFP), Cre recombinase) driven by glial fibrillary acidic protein (GFAP) promoter.

In some embodiments, glial cells used in the methods are glial cells isolated from brain. In some embodiments, astrocytes used in the methods are star-shaped glial cells in brain or spinal cord.

The “effective amount” of the negative regulator or the positive regulator in contact with the non-neuronal cells refers to the quantity of the negative regulator or the positive regulator that is capable of converting the non-neuronal cells into neurons, or refers to the quantity of the carrier containing negative regulator or positive regulator that is capable of converting the non-neuronal cells into neurons. As used herein, “active fragment”, and “functional fragment” have the same meaning and refer to a truncated fragment of a gene or protein that has the same or similar function as the full-length gene or protein. For example, “functional fragment of SEQ ID NO: 36” refers to a truncated fragment of SEQ ID NO: 36 that exhibits similar functionality to the sequence shown in SEQ ID NO: 36. This truncated fragment can exert the same or similar positive regulatory effect as SEQ ID NO: 36. NO: 36, promoting the trans-differentiation of non-neuronal cells into neurons.

As used herein, the inhibitor of a negative regulator gene may be an RNA interfering agent that contains RNA and mediates the targeted cleavage of RNA transcripts through the RNA-induced silencing complex (RISC) pathway. RNA interfering agent directs the sequence-specific degradation of mRNA through the process of RNA interference (RNAi). RNA interfering agents can inhibit the expression of one or more negative regulatory gene in cells. RNA interfering agents include, but are not limited to, “small interfering RNA (siRNA)”, “endoribonuclease-prepared siRNA (e-siRNA)”, “short hairpin RNA (shRNA)” and “small time-regulated RNA” (stRNA), “dicer-processed siRNA (d-siRNA)”, aptamers, oligonucleotides, and other synthetic nucleic acids containing at least one uracil base. In some embodiments, the said RNA interfering agents are delivered by carrier, which include but are not limited to replication-defective or replication-competent viral vector (e.g., adenovirus, lentivirus, gamma retrovirus, adeno-associated virus, etc.).

In some embodiments, this invention provides methods of generating neurons in vivo. Exemplary methods include administering a positive regulator or a negative regulator to the nervous system of a subject, such as the brain, eye, or spinal cord, and reprogramming the non-neuronal cells into functional neurons.

The term “expression vectors” refers to the vectors that add expression elements (such as promoters, RBS, GOI, terminators, etc.) in the basic skeleton of the cloning vector to enable the expression of the target gene. Constructing an expression vector can express the target gene in the recipient cells and perform a function.

### “Negative Regulatory Gene” and “Negative Regulator”

As used herein, “negative regulator gene” refers to a gene that can increase, promote or improve the trans-differentiation of non-neuronal cell into neuron when the expression of the gene, or the expression of its mRNA, or the expression of the protein encoded by the gene is reduced or inhibited. Exemplary of negative regulator gene is: RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B, etc.

As used herein, the “negative regulator” refers to a regulator that is capable of reducing or inhibiting the expression of negative regulator gene, or the mRNA expression of the negative regulator gene, or the expression of the protein encoded by negative regulator gene. In some specific embodiments, a negative regulator can be a gene editing tool or an epigenetic regulatory tool capable of reducing the expression of negative regulator gene; it can also be an inhibitor of negative regulator gene, an activity inhibitor of negative regulator gene, or a degradation activator of the protein encoded by negative regulatory gene.

In some embodiments, “negative regulator” can be an inhibitory antibody of negative regulator gene; or a small molecule inhibitor of negative regulator gene; or an inhibitory mRNA, microRNA, siRNA, shRNA, antisense oligonucleotide, binding protein or protein domain, polypeptide, nucleic acid aptamer, or PROTAC of the negative regulatory gene; or inhibitory binding protein or ligand of the negative regulatory gene.

In some specific embodiments, the gene editing tool used as a “negative regulator” can be a CRISPR gene editing tool, including a gene editing protein and a gRNA targeting a negative regulatory gene. In specific embodiments, the gene editing protein can be a DNA editing protein, such as Cas9, or an RNA editing protein, such as Cas13.

In some specific embodiments, the gene editing tool used as a “negative regulator” can be a gene editing tool containing zinc finger nucleases or TALENs (transcription activator-like (TAL) effector nucleases).

In some embodiments, the “negative regulator” may be an epigenetic regulatory tool. Epigenetic regulatory tool refers to protein, gene, small molecular compound, etc., that is used to regulate the amount and function of nucleic acid or protein in cells through epigenetic modifications (such as methylation, acetylation, phosphorylation, chromatin conformational changes, etc.), without altering the nucleotide sequence of the target gene. For example: the epigenetic regulatory protein fusion such as forming by the fusion of mutated CRISPR/Cas system and KRAB or DNM13A.

### “Positive Regulatory Gene” and “Positive Regulator”

As used herein, the “positive regulatory gene” refers to a gene that can increase, promote or improve the trans-differentiation of non-neuronal cells into neurons when the expression of the gene, or the expression of its mRNA, or the protein encoded by the gene increase or activate. Exemplary of positive regulatory gene is: DPYSL2, BAF45b, SCF, HuB, HuC, HuD, CYP1B1, or BTRC.

As used herein, the “positive regulator” refers to a regulator that is capable of increasing or activating the expression of positive regulator gene, or the mRNA expression of the positive regulator gene, or the expression of the protein encoded by the positive regulator gene. In some embodiments, the positive regulator can be an epigenetic regulatory tool that can improve the expression of positive regulatory gene, an expression activator of positive regulatory gene, a degradation inhibitor of the protein encoded by positive regulatory gene, a mRNA stabilizer of positive regulatory gene, or exogenous positive regulatory gene or its functional fragment.

In some embodiments, the “positive regulator” can be: an agonistic antibody of positive regulator gene; or a small molecule agonist of positive regulator gene; or an agonistic binding protein or ligand of positive regulator gene; or an inhibitor of the competitive gene for a positive regulatory gene.

Diseases Associated with Neuronal Function Loss or Neuronal Death

in this disclosure, diseases associated with neuronal function loss or neuronal death may be Parkinson's disease, visual system diseases related to the function loss or death RGC or photoreceptor cell, Alzheimer's disease, brain injury, Huntington's disease, epilepsy, depression, sleep disorder, cerebral ischemia, motor neuron disease, amyotrophic lateral sclerosis, spinal muscular atrophy, ataxia, PloyQ disease, schizophrenia, addiction, Pick's disease, blindness caused by the impairment or death of RGC or photoreceptor cell, macular degeneration, retinitis pigmentosa, deafness, nyctalopia, color blindness, hereditary blindness, congenital amaurosis, etc. In some embodiments, the disease related to neuronal function loss or neuronal death preferably selects from Parkinson's disease and a visual system disease related to function loss or death of RGC or photoreceptor cell, for example: selects from the disease related to dopamine neuronal function loss or death, vision impairment associated with loss or death of retinal ganglion cells or photoreceptor cells.

The visual system disease related to function loss or death of RGC or photoreceptor cell preferably selects from: vision impairment caused by the death of RGC cell or photoreceptor cell, glaucoma, age-related RGC pathology, optic nerve injury, age-related macular degeneration (AMD), diabetes-related retinopathy, retinal ischemia or hemorrhage, Leber's hereditary optic neuropathy, or a combination thereof; the visual system disease related to function loss or death of photoreceptor cell preferably selects from: photoreceptor cell degeneration or death caused by injury or degenerative disease, macular degeneration, retinitis pigmentosa, diabetes-related blindness, nyctalopia, color blindness, hereditary blindness, congenital amaurosis, or combinations thereof.

### Astrocytes

Astrocytes are the most numerous type of cells in the mammalian brain. They perform many functions, including biochemical support (such as forming the blood-brain barrier), providing nutrients to neurons, maintaining ion balance in extracellular, and participating in repair and scar formation after brain and spinal cord injury. Astrocytes can be divided into two types based on the content of glial filaments and the shape of their cell-process: fibrous astrocytes are mostly distributed in the white matter of brain and spinal cord, with slender cell-processes and fewer branches, the cytoplasm contains a large number of glial filaments; protoplasmic astrocytes are mostly distributed in the gray matter, with thick and short cell-processes and many branches.

Astrocytes that can be used in this disclosure are not particularly limited and include various astrocytes derived from the central nervous system of mammal, such as from the striatum, substantia nigra, midbrain ventral tegmental area, hypothalamus, spinal cord, dorsal midbrain, or cerebral cortex, preferably, derived from the striatum and substantia nigra.

### Neurons

In this disclosure, functional neurons may refer to neurons capable of sending or receiving information through chemical or electrical signals. In some embodiments, functional neurons exhibit one or more functional properties of mature neurons present in the normal nervous system, including but not limited to: excitability (e.g., the ability to exhibit action potentials, e.g., rapid rise and subsequent fall)(voltage or membrane potential across the cell membrane). Neurons are capable of forming synaptic connections with other neurons, performing presynaptic neurotransmitter release, and performing postsynaptic responses (e.g., excitatory postsynaptic currents or inhibitory synaptic after-touch current).

In some embodiments, functional neurons are characterized by expressing one or more markers of functional neurons, including but not limited to express synaptophysin, synapsin, glutamate decarboxylase 67 (GAD67), glutamine acid decarboxylase 65 (GAD65), parvalbumin, dopamine- and cAMP-regulated neuronal phosphoprotein 32 (DARPP32), vesicular glutamate transporter 1 (vGLUT1), vesicular glutamate transporter 2 (vGLUT2), acetylcholine, tyrosine hydroxylase (TH), dopamine, vesicular GABA transporter (VGAT) and gamma-aminobutyric acid (GABA).

In some embodiments, functional neurons can be dopaminergic neurons, 5-HT neurons, NE neurons, ChAT neurons, GABA neurons, glutamatergic neurons, motor neurons, photoreceptor cells (e.g. rods and cones), retinal ganglion cells (RGC), cochlear neurons (such as cochlear spiral ganglion cells and vestibular neurons), or medium spiny neurons (MSN) or combinations thereof, preferably can be dopaminergic neurons, retinal ganglion cells, and photoreceptor cells.

In some embodiments, the functional neurons are mammalian neurons, e.g., are the neurons from human, non-human primate, rat, mouse.

### Dopaminergic Neuron or Dopamine Neuron

Dopaminergic neurons are neurons that contain and release dopamine (DA) as the neurotransmitter. Dopamine belongs to catecholamine neurotransmitter that plays an important biological role in the central nervous system. Dopaminergic neurons in brain mainly concentrate in the substantia nigra pars compacta (SNc) of midbrain, ventral tegmental area (VTA), hypothalamus and the area around encephalocele. Many experiments have confirmed that dopaminergic neurons are closely related to various diseases of human, the most typical disease is Parkinson's disease.

### Gene Editing Tool

Gene editing tool refers to the process of modifying the target gene through gene editing technology, including the insertion, deletion, or replacement in the genes, thereby changing their genetic information and phenotypic characteristics.

In this disclosure, available gene editing tool include, but are not limited to: CRIPSR gene editing tool (CRISPR/Cas system), zinc finger nuclease gene editing tool (ZFN system), TALENs gene editing tool (transcription activator-like (TAL) effector nucleases).

In some embodiments, the gene editing tool include DNA gene editing tool or RNA gene editing tool.

In a preferred embodiment, the gene editing tool used in this disclosure is a CRIPSR gene editing tool. In some embodiments, the CRIPSR gene editing tool includes encoding nucleic acid of Cas protein (also referred as Cas enzyme in this disclosure) or its functional domain, and gRNA targeting the target gene. gRNA can guide the Cas protein to target the target gene and perform gene editing.

In some specific embodiments, the Cas protein is the protein or its mutant from the family of Cas9, Cas12 (e.g., Cas12a, Cas12b, Cas12c, Cas12d, etc.), Cas13, Cas14, CasX, or CasY. In some specific embodiments, the Cas protein is Cas13a, Cas13d, Cas13X, Cas13a, Cas13b (for example: Cas13b-t1, Cas13b-t2, Cas13b-t3, etc.), Cas13c, or Cas13Y. In some embodiments, the Cas13d is CasRx.

In a preferred embodiment, the gene editing tool can reduce or silence the expression of the negative regulatory genes involved in this application.

In some cases, gene editing tools can be delivered via carrier, for example, via adeno-associated virus to deliver gRNA and gene editing protein (e.g., CasRx) provided herein.

### Cas Protein

In this disclosure, the nucleotide of the Cas protein can be obtained through genetic engineering techniques, such as genome sequencing, polymerase chain reaction (PCR), etc., and its amino acid sequence can be derived from the nucleotide sequence. The sources of the wild-type Cas protein include but are not limited to: Ruminococcus flavefaciens, Streptococcus pyogenes, Staphylococcus aureus, Acidaminococcus sp, Lachnospiraceae (Lachnospiraceaeacterium).

In a preferred embodiment of this disclosure, the Cas protein is capable of editing DNA.

In a preferred embodiment of this disclosure, the Cas protein is capable of editing RNA. For example, the Cas protein can be an RNA-targeting gene editing protein such as Cas13d, Cas13e, Cas13a, Cas13b, Cas13c, Cas13f, etc.

### Polypeptide, Protein, and Polynucleotide

In this disclosure, the terms “protein in this disclosure”, “protein”, and “polypeptide” are used interchangeably and may refer to the protein or polypeptide having the amino acid sequence of RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B, BTRC, CYP1B1, DPYSL2, BAF45b, SCF, HuB, HuC, or HuD. They include the protein with or without the starting methionine. Furthermore, the term also includes the full length of said protein or fragment thereof. The proteins referred to in this disclosure include their complete amino acid sequences, their secreted proteins, their mutants, and their functional fragments.

The full names of RCOR1, RCOR2 and RCOR3 are REST corepressor 1, REST corepressor 2 and REST corepressor 3. They have similar functions; the full name of HDAC1 is histone deacetylase 1, the full name of HDAC2 is histone deacetylase 2, both of which have similar functions; the full name of KDM1A is lysine-specific demethylase 1A, also known as lysine-specific histone demethylase 1A (LSD1). Sin3a (SIN3 transcriptional regulatory protein family member A) and Sin3b (SIN3 transcriptional regulatory protein family member B) have similar functions.

In this disclosure, the terms “gene”, “polynucleotide” and “nucleic acid” are used interchangeably and all may refer to nucleic acid sequence having the nucleotide sequence of RCOR1, RCOR2, RCOR3, Sin3a, Sin3b, HDAC1, HDAC2, KDM1A, PHF21A, BAF53a, G9a, USP14, HuR, BrG1, EZH2, CDYL, HMG20B, CYP1B1, BTRC, DPYSL2, BAF45b, SCF, HuB, HuC, or HuD. The full genome length of the human RCOR1 gene is 173,980 bp (NCBI GenBank ID No. is 23186). The full genome length of mouse RCOR1 gene is 76536 bp (NCBI GenBank ID No. is 217864). The full genome length of the human RCOR2 gene is 5935 bp (NCBI GenBank ID No. is 283248). The full genome length of mouse RCOR2 gene is 7832 bp (NCBI GenBank ID No. is 104383). The full genome length of the human RCOR3 gene is 57020 bp (NCBI GenBank ID No. is 55758). The full genome length of mouse RCOR3 gene is 39526 bp (NCBI GenBank ID No. is 214742). The full genome length of the human Sin3a gene is 86441 bp (NCBI GenBank ID No. is 25942). The full genome length of the mouse Sin3a gene is 56382 bp (NCBI GenBank ID No. is 20466). The full genome length of the human Sin3b gene is 50958 bp (NCBI GenBank ID No. is 23309). The full genome length of mouse Sin3b gene is 34934 bp (NCBI GenBank ID No. is 20467). The full genome length of the human HDAC1 gene is 41546 bp (NCBI GenBank ID No. is 3065). The full genome length of mouse HDAC1 gene is 26543 bp (NCBI GenBank ID No. is 433759). The full genome length of the human HDAC2 gene is 38121 bp (NCBI GenBank ID No. is 3066). The full genome length of the mouse HDAC2 gene is 27593 bp (NCBI GenBank ID No. is 15182). The full genome length of the human KDM1A gene is 64249 bp (NCBI GenBank ID No. is 23028). The full genome length of the mouse KDM1A gene is 52284 bp (NCBI GenBank ID No. is 99982). The full genome length of the human PHF21A gene is 192136 bp (NCBI GenBank ID No. is 51317). The full genome length of mouse PHF21A gene is 180916 bp (NCBI GenBank ID No. is 192285). The full genome length of human BAF53a gene is 25482 bp (NCBI GenBank ID No. is 86). The full genome length of the mouse BAF53a gene is 18428 bp (NCBI GenBank ID No. is 56456). The full genome length of the human G9a gene is 17940 bp (NCBI GenBank ID No. is 10919). The full genome length of the mouse G9a gene is 15624 bp (NCBI GenBank 10 No. is 110147). The full genome length of the human USP14 gene is 56073 bp (NCBI GenBank ID No. is 9097). The full genome length of mouse USP14 gene is 36535 bp (NCBI GenBank ID No. is 59025). The full genome length of human HuR gene is 47069 bp (NCBI GenBank ID No. is 1994). The full genome length of mouse HuR gene is 40324 bp (NCBI GenBank ID No. is 15568). The full genome length of the human BrG1 gene is 101,279 bp (NCBI GenBank ID No. is 6597). The full genome length of the mouse BrG1 gene is 88150 bp (NCBI GenBank ID No. is 20586). The full genome length of the human EZH2 gene is 76971 bp (NCBI GenBank ID No. is 2146). The full genome length of the mouse EZH2 gene is 65102 bp (NCBI GenBank ID No. is 14056). The full genome length of the mouse CDYL gene is 214247 bp (NCBI GenBank ID No. is 12593). The full genome length of the human CDYL gene is 249,407 bp (NCBI GenBank ID No. is 9425). The full genome length of the human HMG20B gene is 6168 bp (NCBI GenBank ID No. is 10362). The full genome length of the mouse HMG20B gene is 4898 bp (NCBI GenBank ID No. is 15353). The full genome length of the human DPYSL2 gene is 144145 bp (NCBI GenBank ID No. is 1808). The full genome length of the mouse DPYSL2 gene is 108178 bp (NCBI GenBank ID No. is 12934). The full genome length of the human BAF45b gene is 18690 bp (NCBI GenBank ID No. is 8193). The full genome length of the mouse BAF45b gene is 13652 bp (NCBI GenBank ID No. is 29861). The full genome length of the human SCF gene is 87679 bp (NCBI GenBank ID No. is 4254). The full genome length of the mouse SCF gene is 84802 bp (NCBI GenBank ID No. is 17311). The full genome length of the human HuB gene is 160503 bp (NCBI GenBank ID No. is 1993). The full genome length of the mouse HuB gene is 188638 bp (NCBI GenBank ID No. is 15569). The full genome length of the human HuC gene is 29721 bp (NCBI GenBank ID No. is 1995). The full genome length of the mouse HuC gene is 37142 bp (NCBI GenBank ID No. is 15571). The full genome length of the human HuD gene is 155718 bp (NCBI GenBank ID No. is 1996). The full genome length of the mouse HuD gene is 148212 bp (NCBI GenBank ID No. is 15572). The full genome length of the human CYP1B1 gene is 8643 bp (NCBI GenBank ID No. is 1545). The full genome length of mouse CYP1B1 gene is 8122 bp (NCBI GenBank ID No. is 13078). The full genome length of human BTRC gene is 203,506 bp (NCBI GenBank 11) No. is 8945). The full genome length of the mouse BTRC gene is 169,664 bp (NCBI GenBank ID No. is 12234).

The similarity between human and mouse of RCOR1 at the DNA level is 88.51%, and the protein sequence similarity is 92.39%. The similarity between human and mouse of RCOR2 at the DNA level is 89.26%, and the protein sequence similarity is 97.51%. The similarity between human and mouse of RCOR3 at the DNA level is 92.38%, and the protein sequence similarity is 96.39%. The similarity between human and mouse of Sin3a at the DNA level is 90.91%, and the protein sequence similarity is 98.04%. The similarity between human and mouse of Sin3b at the DNA level is 86.61%, and the protein sequence similarity is 87.91%. The similarity between human and mouse of HDAC1 at the DNA level is 90.85%, and the protein sequence similarity is 99.38%. The similarity between human and mouse of HDAC2 at the DNA level is 91.62%, and the protein sequence similarity is 99.59%. The similarity between human and mouse of KDM1A at the DNA level is 89.75%, and the protein sequence similarity is 97.72%. The similarity between human and mouse of PHF21A at the DNA level is 92.88%, and the protein sequence similarity is 94.20%. The similarity between human and mouse of BAF53a at the DNA level is 88.60%, and the protein sequence similarity is 98.84%. The similarity between human and mouse of G9a at the DNA level is 87.74%, and the protein sequence similarity is 94.75%. The similarity between human and mouse of USP14 at the DNA level is 91.31%, and the protein sequence similarity is 96.76%. The similarity between human and mouse of HuR at the DNA level is 91.03%, and the protein sequence similarity is 98.47%. The similarity between human and mouse of BrG1 at the DNA level is 89.00%, and the protein sequence similarity is 95.47%. The similarity between human and mouse of EZH2 at the DNA level is 91.98%, and the protein sequence similarity is 97.60%, The similarity between human and mouse of CDYL at the DNA level is 86.77%, and the protein sequence similarity is 93.12%. The similarity between human and mouse of HMG20B at the DNA level is 85.53%, and the protein sequence similarity is 93.69%. The similarity between human and mouse of DPYSL2 at the DNA level is 91.05%, and the protein sequence similarity is 97.64%. The similarity between human and mouse of BAF45b at the DNA level is 93.39%, and the protein sequence similarity is 97.69%. The similarity between human and mouse of SCF at the DNA level is 89.44%, and the protein sequence similarity is 82.78%. The similarity between human and mouse of HuB is 95.46%, and the protein sequence similarity is 99.72%. The similarity between human and mouse of HuC at the DNA level is 91.94%, and the protein sequence similarity is 99.46%. The similarity between human and mouse of HuD at the DNA level is 94.64%, and the protein sequence similarity is 99.21%. The similarity between human and mouse of CYP1B1 at the DNA level is 82.96%, and the protein sequence similarity is 81.03%. The similarity between human and mouse of BTRC at the DNA level is 90.87%, and the protein sequence similarity is 98.68%. It should be understood that substitutions of nucleotides in codons are acceptable when encoding the same amino acid. It should also be understood that nucleotide changes are also acceptable if the substitution of nucleotides leading to a conservative amino acid substitution.

The encoding nucleic acid sequence can be constructed based on the amino acid fragment is obtained, and a specific probe can be designed based on the nucleotide sequence. The full-length nucleotide sequence or its fragments can usually be obtained by PCR amplification, recombination or artificial synthesis. For the PCR amplification, primers can be designed based on the nucleotide sequences disclosed in this disclosure, especially the open reading frame sequence. Then the cDNA libraries commercially available or the cDNA libraries prepared through the methods known to those skilled in the art is used as a template to amplify the relevant sequence. If the sequence is long, it is often necessary to perform two or more rounds of PCR amplification, followed by the assembly of the amplified fragments in the correct order.

Once the relevant sequence is obtained, they can be obtained in large quantities using recombinant methods. This is usually done by cloning them into a vector, transferring into cells, and then isolating the relevant sequence from the proliferated host cells through the conventional methods.

In addition, artificial synthesis methods can also be used to synthesize relevant sequences, especially when the fragment length is short. Usually, fragments with long sequences are obtained by synthesizing multiple small fragments first, and then linking them together.

At present, the DNA sequence encoding the protein (or its fragments, derivatives) of this disclosure can be obtained entirely through chemical synthesis.

The DNA sequence can then be introduced into various DNA molecules (e.g., vectors) and cells known in the art.

The polynucleotide sequence of this disclosure may be used to express or produce recombinant polypeptide by conventional recombinant DNA techniques. Generally speaking, the steps are as follows:
(1). Using the polynucleotide (or variant) encoding the polypeptide of this disclosure or the recombinant expression vector containing the polynucleotide to transfect or transduce into a suitable host cell;
(2). Culturing the host cells in appropriate culture media;
(3). Separating and purifying the proteins from culture media or cells.

In this disclosure, polynucleotide sequences can be inserted into recombinant expression vectors. In short, any vector and carrier can be used as long as it can replicate and be stable in the host. An important feature of expression vectors is that they usually contain origin of replication, promoter, marker gene, and translation control element.

Methods well known to those skilled in the art can be used to construct expression vectors containing the coding DNA sequences for RCOR1, RCOR2, RCOR3, Sin3a, Sin3b, HDAC1, HDAC2, KDM1A, PHF21A, BAF53a, G9a, USP14, HuR, BrG1, EZH2, CDYL, CYP1B1, BTRC, HMG20B, DPYSL2, BAF45b, SCF, HuB, HuC, or HuD, along with appropriate transcriptional/translational control signals. These methods include recombinant DNA technology in vitro, DNA synthesis technology, recombination technology in vivo, etc. The DNA sequence can be operably linked to an appropriate promoter in an expression vector to direct mRNA synthesis. The expression vector also includes ribosome binding site for translation initiation and transcription terminator.

In addition, the expression vector preferably contains one or more selectable marker genes to provide phenotypic traits for selection of host cells transfection, such as dihydrofolate reductase, neomycin resistance, and green fluorescent protein (GFP) for eukaryotic cell culture, or tetracycline or ampicillin resistance for E. coli.

Vectors containing appropriate DNA sequences as described above and appropriate promoter or control sequences can be used to transfect appropriate host cells to enable expression of proteins.

The host cell can be prokaryotic cell, such as bacterial cell; or lower eukaryotic cell, such as yeast cell; or higher eukaryotic cell, such as mammalian cell. Representative examples include: Escherichia coli, bacterial cell of Streptomyces; fungal cell such as yeast; plant cell; insect cell; animal cell, etc.

Transfection of host cells with recombinant DNA can be performed using conventional techniques well known to those skilled in the art. If the host is prokaryote such as E. coli, competent cells capable of absorbing DNA can be harvested after the exponential growth phase. The method of CaCl₂ could be used.

The procedures used are well known in the art. Another approach is the method using MgCl₂. If necessary, transfection can also be performed by electroporation. If the host is eukaryote, the following DNA transfection methods can be used: calcium phosphate co-precipitation, conventional mechanical methods such as microinjection, electroporation, liposome packaging, etc.

The obtained transformants can be cultured using conventional methods to express the polypeptide encoded by the gene in this disclosure. Depending on the host cells used, the culture medium can be selected from various conventional medium. Cultivate is carried out under conditions suitable for the growth of host cell. After the host cells growing to an appropriate cell density, the selected promoter is induced using an appropriate method (such as temperature shift or chemical induction), and the cells are cultured for a further period of time.

In the aforementioned methods, the recombinant polypeptide can be expressed in the cell, or on the cell membrane, or secreted outside the cell. If necessary, the recombinant protein can be isolated and purified through any separation methods by utilizing its physical, chemical and other properties. These methods are well known to those skilled in the art. Examples of these methods include, but not limited to: conventional renaturation processes, treatment with protein precipitating agents (salting-out method), centrifugation, osmotic lysis, ultrafiltration treatment, ultracentrifugation, molecular sieve chromatography (gel filtration), adsorption chromatography, ion exchange chromatography, high performance liquid chromatography (HPLC), and other liquid chromatography techniques, as well as combinations of these methods.

### Adeno-Associated Virus

Due to the characteristics as the smaller size compared to other viral vectors, non-pathogenic, and ability to transfect both dividing and non-dividing cells, gene therapy methods for genetic diseases based on Adeno-associated virus (AAV) have received considerable attention.

Adeno-associated virus (AAV) belongs to the family Parvoviridae, genus Dependovirus, which is a type of single-stranded DNA defective virus with the simplest structure currently discovered. It requires a helper virus (usually an adenovirus) to participate in replication. It encodes the cap and rep genes in two terminal inverted repeats (ITRs). ITRs play a decisive role in virus replication and packaging. The cap gene encodes the viral capsid protein, and the rep gene participate in virus replication and integration. AAV can infect a variety of cells.

Recombinant adeno-associated virus vector (rAAV) is derived from non-pathogenic wild-type adeno-associated virus. Due to its excellent safety, wide range of host cells (both dividing and non-dividing cells), low immunogenicity, prolonged expression of exogenous genes in vivo, and other characteristics, it is regarded as one of the most promising gene transfer vectors and has been widely used in gene therapy and vaccine research around the world. After more than 10 years of research, the biological properties of recombinant adeno-associated viruses have been deeply understood, especially a lot of data has been accumulated for their application effects in various cells, tissues and in vivo experiments. In medical research, rAAV is used in the study of gene therapy for a variety of diseases (including in vivo and in vitro experiments); at the same time, as a unique gene transfer vector, it is also widely used in gene function research, construction of disease models, and preparation of the mouse knocking down gene.

In a preferred embodiment of this disclosure, the vector is a recombinant AAV vector. AAVs are relatively small DNA viruses that can integrate into the genome of the infected cells with a stable and site-specific manner. They are able to infect a large range of cells without any impact on cell growth, morphology or differentiation, and they do not appear to be involved in human pathology. The AAV genome has been cloned, sequenced and characterized. Each end of the AAV genome contains an inverted terminal repeat (ITR) region of about 145 bases, which serves as the origin of virus replication. The rest of the genome is divided into two important regions with capsidation functions, which comprise: the left portion of the genome containing the rep genes involved in viral replication and viral gene expression and the right portion of the genome containing the cap gene encoding the viral capsid protein.

AAV vectors can be prepared using standard methods in the art. Adeno-associated virus of any serotype is suitable. Methods for purifying vectors can be found, for example, in U.S. Pat. Nos. 6,566,118, 6,989,264, and 6,995,006, the disclosures of which are incorporated herein by reference in their entirety. The preparation of heterozygous vectors is described, for example, in PCT application No. PCT/US2005/027091, the disclosure of which is incorporated herein by reference in its entirety. The use of the vectors derived from AAV for transporting genes in vitro and in vivo has been described (see, for example, international patent application publication Nos. WO91/18088 and WO93/09239; U.S. Pat. Nos. 4,797,368, 6,596,535 and 5,139,941, and European Patent No. 0488528, each of which is incorporated herein by reference in its entirety. These patent publications describe various AAV-derived constructs in which the rep and/or cap genes are deleted and replaced by genes of interest, as well as the use of these constructs for delivering the gene of interest in vitro (into cultured cells) or in vivo (directly into organisms). Replication-defective recombinant AAVs can be prepared by co-transfecting the following plasmid into a cell line infected with a human helper virus (such as adenovirus): a plasmid containing the nucleic acid sequence of interest flanked by two AAV inverted terminal repeat (ITR) regions, and a plasmid carrying the AAV capsid genes (rep and cap genes). The resulting AAV recombinants are then purified using standard techniques.

In some embodiments, the recombinant vector is encapsidated into viral particles (such as AAV viral particles including but not limited to AAV1, AAV2, AAV3, AAV4, AAV5, AAV6, AAV7, AAV8, AAV9, AAV10, AAV11, AAV12, AAV13, AAV14, AAV15 and AAV16). Accordingly, this disclosure includes recombinant virions (recombinant because they contain recombinant polynucleotides) containing any of the vectors described herein. Methods of producing such virions are known in the art and are described in U.S. Pat. No. 6,596,535.

In some cases, the AAV vector may be modified to include a modified VP protein (e.g., the modified the AAV vectors including VP1 protein, VP2 protein or VP3 protein). In one aspect, the AAV vector is a recombinant AAV (rAAV) vector. rAAV can be composed of capsid sequence and structure that is substantially similar to that found in wild-type AAV (wtAAV). However, rAAV encapsulates a genome that is essentially lacks AAV protein coding sequences and has a therapeutic gene expression cassette designed in its place, such as the subject polynucleotide. In some cases, virally derived sequences may be ITRs that may be required to direct genome replication and packaging during vector production. Suitable AAV vectors may be selected from any AAV serotype or combination of serotypes. For example, the AAV vector can be any of the following: AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12. AAV.HSC13, AAV.HSC14, AAV.HSC15, AAV.HSC16 and AAVhu68, or any combination thereof. In some cases, the vector is selected based on its natural orientation. In some cases, the vector serotype is selected based on its ability to cross the blood-brain barrier. AAV9 and AAV10 have been shown to cross the blood-brain barrier to transduce neurons and glial cells. In one aspect, the AAV vector is AAV2, AAV5, AAV6, AAV8 or AAV9. In some cases, the AAV vector is a chimera of at least two serotypes. In one aspect, the AAV vectors are serotypes AAV2 and AAV5. In some cases, chimeric AAV vectors include rep and ITR sequences from AAV2 and cap sequences from AAV5. In some cases, chimeric AAV vectors include rep and ITR sequences from AAV2 and cap sequences from any other AAV serotype. In some embodiments, AAV vectors may be self-complementary. In some cases, AAV vectors may include inverted terminal repeats. In other cases, AAV vectors may include inverted terminal repeat (scITR) sequences with mutated terminal unwinding sites. In some cases, rep, cap and ITR sequences from all the different AAV serotypes provided herein can be mixed and matched. In some cases, the AAV vector is from an adeno-associated virus with a serotype selected from: AAV I, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some cases, the vector can be a recombinant AAV (rAAV) vector, a hybrid AAV vector, a chimeric AAV vector, a self-complementing AAV (scAAV) vector, a single-stranded AAV, or any combination thereof. In some cases, the AAV vector includes a genome comprising replicate genes and inverted terminal repeats from a first AAV serotype and capsid proteins from a second AAV serotype. In some cases, the AAV vector may be chimeric and may be: an AAV2/S vector, an AAV2/6 vector, an AAV2/7 vector, an AAV2/8 vector, or AAV2/9 vector. In some cases, the inverted terminal repeats of the AAV vector include 5′ inverted terminal repeats, 3′ inverted terminal repeats, and mutated inverted terminal repeats. In some cases, the mutated inverted terminal repeat lacks a terminal unwinding site. In some cases, suitable AAV vectors can be further modified to encompass modifications such as in the capsid or rep protein. Modifications may also include deletions, insertions, mutations, and combinations thereof. In some cases, the carrier is modified to reduce immunogenicity, allowing for repeated administration. In some cases, when repeated administration is performed to reduce and/or eliminate immunogenicity, the serotype of the vector used changes.

### Pharmaceutical Composition

Utilizing the content of this disclosure, through various conventional screening methods, it is possible to screen a negative regulator that can inhibit the gene or protein of RCOR1, RCOR2, RCOR3, Sin3a, Sin3b, HDAC1, HDAC2, KDM1A, PHF21A, BAF53a, G9a, USP14, HuR, BrG1, EZH2, CDYL, or HMG20B.

In some specific embodiments, the inhibitor is one or several types of negative regulators, which inhibit the expression of negative regulatory genes, or inhibit the mRNA level of negative regulatory genes, or inhibit the level of the proteins encoded by negative regulatory genes.

Utilizing the content of this disclosure, through various conventional screening methods, it is possible to screen a positive regulator that can enhance the gene expression level, mRNA level, protein expression level, or protein activity of DPYSL2, BAF45b, SCF, HuB, HuC, HuD, CYP1B1, or BTRC.

In some specific embodiments, the agonists are one or more positive regulators that can increase the expression of positive regulatory gene, or elevate the mRNA level of positive regulatory gene, or augment the level of encoded proteins of positive regulatory gene.

Negative or positive regulators used in this disclosure may be the substance that can reduce or eliminate the expression and/or activity of the gene, its RNA (e.g., mRNA), or its encoded protein at the DNA, RNA, or protein level.

In some specific embodiments, the negative regulator includes inhibitory antibodies of negative regulatory gene; or small molecule inhibitors of negative regulatory gene; or inhibitory mRNA, microRNA, siRNA, shRNA, antisense oligonucleotide, binding proteins or protein domains, polypeptides, nucleic acid aptamers, or PROTACs of negative regulatory gene; or inhibitory binding proteins or ligands of negative regulatory gene.

In some embodiments, negative regulator of RCOR1 in this disclosure include inhibitor targeting a target sequence comprising SEQ ID NO: 68. Negative regulator of RCOR2 in this disclosure include inhibitor targeting a target sequence comprising SEQ 10 NO:69. Negative regulator of RCOR3 in this disclosure include inhibitor targeting a target sequence comprising SEQ ID NO:70. Negative regulator of Sin3a in this disclosure include inhibitor targeting a target sequence comprising SEQ ID NO:71. Negative regulator of Sin3b in this disclosure include inhibitor targeting a target sequence comprising SEQ ID NO:72. Negative regulator of HDAC1 in this disclosure include inhibitor targeting a target sequence comprising SEQ ID NO:73. Negative regulator of KDM1A in this disclosure include inhibitor targeting a target sequence comprising SEQ ID NO:74. Negative regulator of HDAC2 in this disclosure include inhibitor targeting a target sequence comprising SEQ ID NO:75. Negative regulator of PHF21A in this disclosure include inhibitor targeting a target sequence comprising SEQ ID NO:76. Negative regulator of BAF53a in this disclosure include inhibitor targeting a target sequence comprising SEQ ID NO:77. Negative regulator of G9a in this disclosure include inhibitor targeting a target sequence comprising SEQ ID NO:78. Negative regulator of USP14 in this disclosure include inhibitor targeting a target sequence comprising SEQ ID NO:79. Negative regulator of HuR in this disclosure include inhibitor targeting a target sequence comprising SEQ ID NO:80. Negative regulator of BrG1 in this disclosure include inhibitor targeting a target sequence comprising SEQ ID NO:81. Negative regulator of EZH2 in this disclosure include inhibitor targeting a target sequence comprising SEQ ID NO:82. Negative regulator of CDYL in this disclosure include inhibitor targeting a target sequence comprising SEQ ID NO:83. Negative regulator of HMG20B in this disclosure include inhibitor targeting a target sequence comprising SEQ ID NO:84.

In some embodiments, the objects of the negative regulator or positive regulator in this disclosure include astrocytes or MG cells.

In some embodiments, the positive regulator can be a substance that increases the expression and/or activity of a positive regulatory gene, or its RNA (such as mRNA), or its encoded protein at the DNA, RNA, or protein level.

In some embodiments, the positive regulator includes: expression vector containing promoter, endogenous expression activator, protein analog or enhancer, epigenetic regulatory tool that can improve the expression of positive regulatory gene, expression activator of positive regulatory gene, degradation inhibitor of protein encoded by positive regulatory gene, mRNA stabilizer of positive regulatory gene, or exogenous positive regulatory gene or the functional fragment of positive regulatory gene.

In a preferred embodiment, the methods and steps of regulating a negative regulator include using an antibody to neutralize the protein, using shRNA or siRNA carried by a virus (such as adeno-associated virus), or using gene editing tool to silence the gene.

The inhibition efficiency of negative regulators to negative regulatory gene is generally at least 50% or more, preferably 60%, 70%, 80%, 90%, or 95% or more. The inhibition efficiency can be controlled and detected based on conventional techniques, such as flow cytometry, fluorescence quantitative PCR or Western blot.

Negative regulators (including antibodies, antisense nucleic acids, gene editing tools and other inhibitors) or positive regulators (including expression vectors containing promoters, endogenous expression activators, protein analogs or enhancers) disclosed herein, when administered therapeutically, can inhibit or enhance the expression and/or activity of the gene or protein, thereby inducing the differentiation of glial cells into neuronal cells, thereby treating the diseases associated with neuronal function loss or death. Generally, these materials may be formulated in a nontoxic, inert, and pharmaceutically acceptable aqueous medium, although the pH may vary depending on the character of the materials being formulated and the condition being treated, the pH is usually about 5-8, preferably is about 6-8. It varies depending on the nature of the treatment and the condition to be treated. The formulated pharmaceutical composition can be administered by conventional routes, including (but not limited to): topical, intramuscular, intracranial, intraocular, intraperitoneal, intravenous, subcutaneous, intradermal, local administration, autologous cell reinfusion after extraction and culture.

This disclosure also provides a pharmaceutical composition, which contains safe and effective amount of the negative regulators or positive regulators of this disclosure and a pharmaceutically acceptable carrier or excipient. Such carriers include, but are not limited to: saline, buffer, glucose, water, glycerol, ethanol, or combinations thereof. The pharmaceutical formulation should match the mode of administration. The pharmaceutical composition of this disclosure can be formulated into an injection form, for example, using physiological saline or an aqueous solution containing glucose and other adjuvants by conventional methods. Pharmaceutical compositions, such as tablets and capsules, can be prepared by conventional methods. Pharmaceutical compositions such as injections, solutions, tablets and capsules should be manufactured under sterile conditions. The dosage of the active ingredient is administered in a therapeutically effective amount, for example, about 1 microgram to 10 mg/kg of body weight per day.

Key advantages of this disclosure include:
(1) This disclosure finds for the first time that reducing the expression or activity of gene or its encoded protein of RCOR1, RCOR2, RCOR3, Sin3a, Sin3b, HDAC1, HDAC2, KDM1A, PHF21A, BAF53a, G9a, USP14, HuR, BrG1, EZH2, CDYL, or HMG20B in non-neuronal cells, particularly glial cells, and/or increasing the expression or activity of gene or its encoded protein of DPYSL2, BAF45b, SCF, HuB, HuC, HuD, CYP1B1, or BTRC in glial cells can induce the differentiation of glial cells into dopaminergic neurons, thereby preventing and/or treating diseases associated with neuronal function loss or death.
(2) Using the negative regulator or positive regulator provided by the present invention can induce the production of dopamine neurons, thus having the potential to treat Parkinson's disease.
(3) This disclosure directly transforms Müller cells into retinal ganglion cells (RGCs) or photoreceptor cells by inhibiting the expression of RCOR1, RCOR2, RCOR3, Sin3a, Sin3b, HDAC1, HDAC2, KDM1A, PHF21A, BAF53a, G9a, USP14, HuR, BrG1, EZH2, CDYL, or HMG20B in the retina and/or enhancing the expression of DPYSL2, BAF45b, SCF, HuB, HuC, HuD, CYP1B1, or BTRC in glial cells.
(4) The regenerated RGC can be integrated into the visual pathway and improve visual function in RGC injury mouse models.

This disclosure will be further elaborated below with reference to specific embodiments. It should be understood that these examples are merely illustrative of the disclosure and are not intended to limit the scope of the disclosure. Experimental methods without specifying specific conditions in the following examples usually follow conventional conditions, such as those described in Sambrook et al., Molecular Cloning: Laboratory Manual (New York: Cold Spring Harbor Laboratory Press, 1989), or according to conditions recommended by the manufacturer. Unless otherwise stated, percentages and parts refer to percentages by weight and parts by weight, respectively.

Unless otherwise stated, the materials and reagents used in the examples of this disclosure are commercially available products.

### General Methods

Animal ethics: The use and care of animals are in compliance with the guiding principles of the Biomedical Research Ethics Committee of the Center for Excellence in Brain Science and Intelligent Technology. Chinese Academy of Sciences.

Guide RNA sequence and AAV expression plasmid construction: In order to overexpress nSR100 in glial cells, the glial cell-specific promoter GFAP is chosen to initiate the expression of nSR100.

Transient transfection of astrocytes and qPCR: Isolate primary astrocytes from mice and seed the astrocytes into a 6-well plate. According to standard procedures, Lipofectamine 3000 (Thermo Fisher Scientific) is used to transiently transfect 3 μg of vectors expressing gRNA-CasRx-GFP. Control plasmid expresses non-targeting gRNA. 1-2 days after transient transfection, GFP-positive cells are collected by fluorescence-activated cell sorting (FACS) and lysed for qPCR analysis. The qPCR analysis method is: Use Trizol (Ambion) to extract RNA and then reversely transcribe the RNA into cDNA using a reverse transcription kit (HiScript Q RT SuperMix for qPCR, Vazyme, Biotech). Tracing the expansion through AceQ qPCR SYBR Green Master Mix (Vazyme, Biotech). The qPCR primers are shown in Table 2 below:

TABLE 2
qPCR primers

| SEQ ID NO. | NAME | SEQUENCE |
|---|---|---|
| SEQ ID | human-GAPDH-F | gtctcctctgacttcaacagcg |
| NO.: 85 | | |
| SEQ ID | human-GAPDH-R | accaccctgttgctgtagccaa |
| NO.: 86 | | |
| SEQ ID | human-RCOR1-F | ggagagcgaggatgaactgg |
| NO.: 87 | | |
| SEQ ID | human-RCOR1-R | gcattggcagaaacagcctc |
| NO.: 88 | | |
| SEQ ID | human-RCOR2-F | acccaaccactgtgtgtcag |
| NO.: 89 | | |
| SEQ ID | human-RCOR2-R | cagcgacttctccacatcgt |
| NO.: 90 | | |
| SEQ ID | human-RCOR3-F | cagaacaaggaacccaggct |
| NO.: 91 | | |
| SEQ ID | human-RCOR3-R | gagtggcaataggggctgtt |
| NO.: 92 | | |
| SEQ ID | human-Sin3A-F | cccaagtgtacaggacggac |
| NO.: 93 | | |
| SEQ ID | human-Sin3A-R | aacccggattgttgccagat |
| NO.: 94 | | |
| SEQ ID | human-Sin3B-F | gacggtgcagaggacttcaa |
| NO.: 95 | | |
| SEQ ID | human-Sin3B-R | tgttcagctgctccttctgg |
| NO.: 96 | | |
| SEQ ID | human-HDACI-F | ctatcgccctcacaaagccaa |
| NO.: 97 | | |
| SEQ ID | buman-HDACI-R | ctgcttgctgtactccgaca |
| NO.: 98 | | |
| SEQ ID | human-KDMIA-F | atttgttcgggcatgttggc |
| NO.: 99 | | |
| SEQ ID | human-KDMIA-R | cggccaacaatcacatcgtc |
| NO.: 100 | | |

Stereotaxic injection: In this study, AAV8 is used to deliver the gene of interest. The titer of AAV-CasRx, AAV-CasRx-gRNA, or AAV-GFAP-positive regulatory gene is greater than 5×10¹² vg/ml (1-3 μl per injection). AAV was injected into the striatum (AP+0.8 mm, ML±1.6 mm and DV-2.8 mm).

Immunofluorescence staining: 6-12 weeks after injection in mice, perfusion is performed, and then the brains are harvested to fix overnight in 4% paraformaldehyde (PFA), Subsequently, the brains are dehydrated in 30% sucrose for at least 12 hours. After embedding, the brains are frozen and sectioned into a slice with 30 μm thickness. Before immunofluorescence staining, brain sections are thoroughly rinsed with 0.1 M phosphate buffer saline (PBS). Primary antibodies: rabbit polyclonal NeuN antibody (Brain, 1:500, #ABN78, Millipore), mouse TH antibody (1:300, MAB318, Millipore). Secondary antibodies: Donkey anti-mouse (#715-545-150, Jackson ImmunoResearch), donkey anti-rabbit (#711-545-152, Jackson ImmunoResearch) are used in this study. After antibody incubation, sections are washed and covered with mounting medium (Life Technology).

Statistical analysis: Error bars are set by s.e.m., and statistical significance (p<0.05) is calculated using unpaired two-tailed t-test or one-way ANOVA. All experiments are randomly formulated, and no statistical methods are used to predetermine the sample size.

For RNA-seq, 293T or N2a cells are cultured in 15-cm culture dishes and transiently transfected with 70 μg of plasmid. The top 20% of GFP and mCherry double-positive N2a cells are collected by FACS, RNA is extracted and converted to cDNA, and then used for whole-transcriptome RNA-seq.

Intravitreal and subretinal injections: As previously described, NMDA and AAV8 are introduced via intravitreal and subretinal injections, respectively.

Subretinal injection: Inject AAV(>1×10¹³ vg/ml) into the eye using a Hamilton syringe (32G needle) under an Olympus microscope. To determine reprogramming in retinas completely, a total of 1 μl of GFAP-GFP-Cre (0.2 μl) and AAV-GFAP-nSR100 or GPAP-CasRx-gRNA (0.8 μl), or GFAP-GPP-Cre (0.2 μl) and GFAP-CasRx (0.8 μl) is delivered to the retina via subretinal injection in Ai9 and C57BL/6 mice aged 5 weeks to 12 months. To determine reprogramming in damaged retinas, dissolve NMDA in PBS to a concentration of 200 mM, and then inject 1.5 μl of NMDA solution into the eyes of 4-8 week old Ai9 mice or 5-6 week old C57BL6 mice via intravitreal injection (for VEP and black-and-white scene preference testing). 2-3 weeks after NMDA injection, each virus combination is co-delivered to the retina via subretinal injection.

Immunofluorescence staining: 1.5-3 months after AAV injection, the eyes, optic nerve and brain are harvested and fixed with 4% paraformaldehyde (PFA) for 2 hours (eyes and optic nerve) or for 24 hours (brain), and then, dehydration is performed in a 30% sucrose solution for 2 hours (for eyes and optic nerves) or 24 hours (for brains). After embedding and freezing, eyes and brains are sectioned at a thickness of 30 μm. Primary antibodies used for immunofluorescence staining: rabbit anti-RBPMS (1:500, 15187-1-AR Proteintech), and secondary antibodies: Jackson ImmunoResearch), Cy TM 5AffiniPure Donkey rabbit anti-IgG (H+L) (1:500, 711-175-152, Jackson ImmunoResearch). After incubating the antibodies, wash the slides with PBS and mount them with anti-fluorescence quencher. Imaging is performed using an Olympus FV3000 microscope.

### EXAMPLES

### Example 1. In Vitro Specific Knockdown Using CasRx

In order to evaluate the knockdown of negative regulatory genes, gRNAs targeting RCOR1, Sin3a, HDAC2, KDM1A, PHF21A, BAF53a, G9a, USP14, HuR, BrG1, EZH2, CDYL, and HMG20B are designed, and CasRx and gRNA are co-transfected into N2A cells.

The nucleotide sequence of gRNAs and target sequences are shown in Table 1 above.

The transfection method is: transiently transfect 4 μg CAG-CasRx-P2A-GFP plasmid and 2 μg U6-gRNA-CMV-mCherry plasmid to determine the inhibitory effect of each gRNA in vitro cell lines. CAG-CasRx-P2A-GFP plasmid is transfected alone and served as the control group. Lipofectamine 3000 (Thermo Fisher Scientific) is used according to standard procedures. Two days after transfection, 30,000 GFP and mCherry double-positive cells are collected from each sample by fluorescence-activated cell sorting (FACS), and lysed for qPCR analysis. The qPCR primers are listed in Table 2.

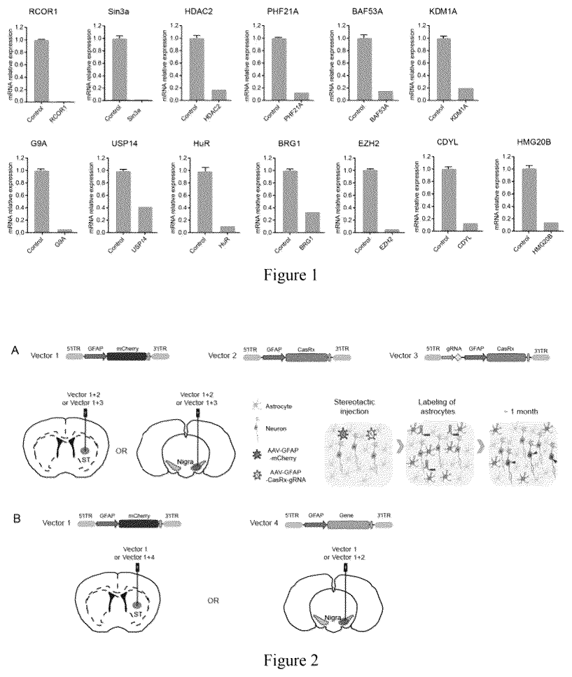

The results are shown in FIG. 1. Co-transfection of gRNA targeting the mRNA of the negative regulatory gene and CasRx can achieve significant knockdown of the target gene in mouse N2A cells.

### Example 2. Study of the Negative Regulatory Genes on the Trans-Differentiation of Astrocytes into Neurons

In order to study whether knockdown of negative regulatory genes can induce the trans-differentiation of astrocytes into neurons or dopaminergic neurons, AAV-GFAP-CasRx-gRNA or control vectors AAV-GFAP-CasRx (without gRNA) and AAV-GFAP-mCherry are injected into the striatum of C57BL/6 mice older than eight weeks. The plasmid designs are shown in FIGS. 2A and B. Inject the combination of vector 1+vector 2 or vector 1+vector 3, wherein, GFAP is an astrocyte-specific promoter, AAV-GFAP-mCherry is used to specifically label astrocytes, AAV-GFAP-CasRx is the control group AAV, and the gRNAs specifically target the negative regulatory gene RCOR1, HDAC2, PHF21A, BAF53a, G9a, USP14, HuR, BrG1, EZH2, CDYL, HMG21B, or KDM1A.

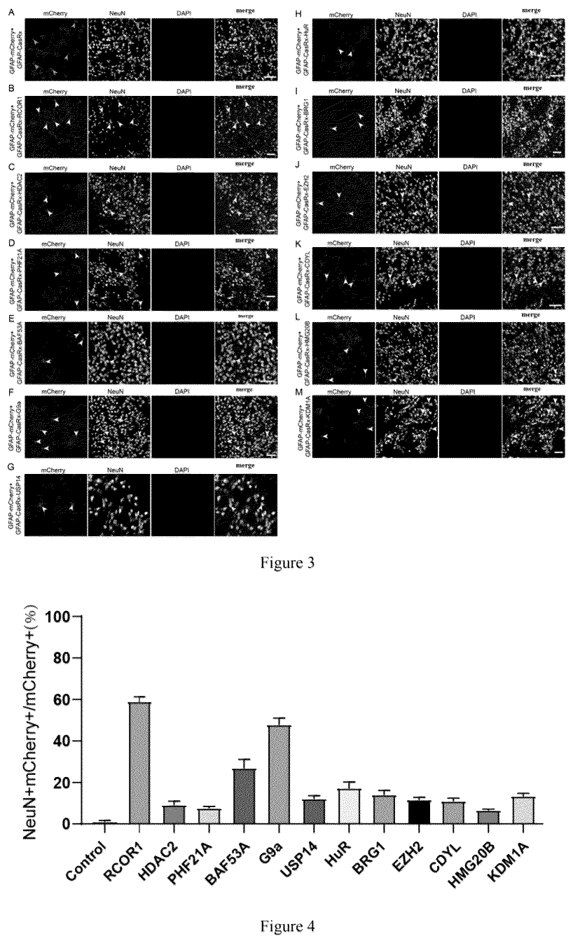

Samples are collected and analyzed 1-3 months after injection. The results are shown in FIG. 3. As shown in FIG. 3A, in the control group injected with GFAP-mCherry+AAV-GFAP-CasRx, mCherry-positive red cells show typical glial cell morphology, and no red cells with obvious neuronal morphology are found. Further immunofluorescence staining with the neuronal-specific marker protein NeuN, it is revealed that almost no red cells in the control group are co-labeled with NeuN, which indicate GFAP-mCherry could specifically label astrocytes and the control group injected with GFAP-mCherry+AAV-GFAP-CasRx could not induce the trans-differentiation of astrocytes to neurons. As shown from FIG. 3B to 3M, in the test groups injected with GFAP-mCherry+AAV-GFAP-CasRx-gRNA, varying number of mCherry-positive cells co-label with NeuN, which indicate that RCOR1, HDAC2, PHF21A, BAF53a, G9a, USP14, HuR, BrG1, EZH2, CDYL, HMG20B, and KDM1A can all induce the trans-differentiation of glial cells into neurons with varying degrees.

FIG. 4 shows the statistics of astrocytes converted into neurons after knocking down different negative regulatory genes. Among them, the higher efficiency is exhibited in inducing the trans-differentiation from glial cells to neurons when knock down the negative regulatory gene RCOR1, G9a, BAF53A, and so on.

### Example 3. Study of Positive Regulatory Genes on the Trans-Differentiation of Astrocytes into Neurons

In order to study the effects of overexpressing positive regulatory genes on trans-differentiation, an attempt is made to overexpress DPYSL2, BAF45b, SCF, HuB, HuC, HuD, CYP1B1, and BTRC in the mouse striatum.

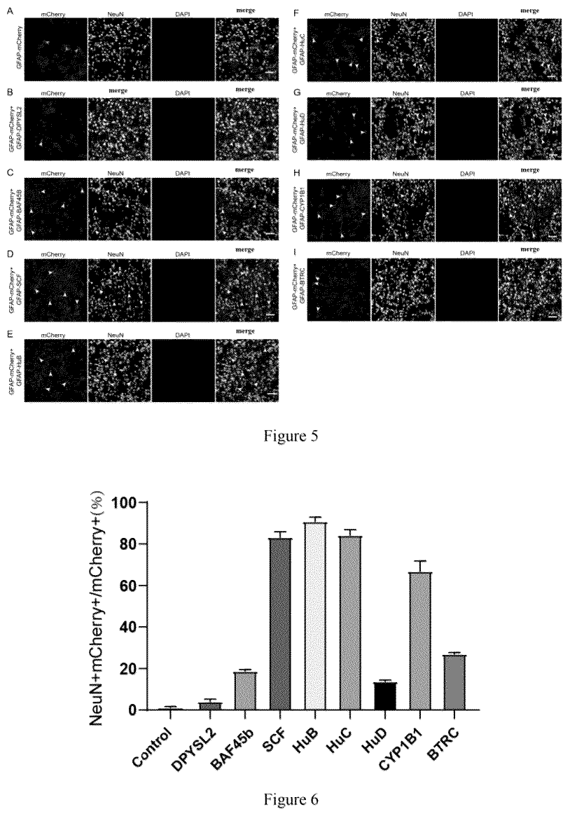

AAVs in the control group or the test group are injected into the striatum or substantia nigra of mice. Among them, vector 1 is AAV-GFAP-mCherry, and the target gene of vector 2 (AAV-GFAP-Gene) is driven to express by the astrocyte-specific promoter GFAP. Similar to the groups aimed at knocking down the expression of the target gene, samples are collected 1-3 months after AAV injection to analyze the efficiency of various targets in inducing glial cells to transdifferentiate into neurons. The results are shown in FIG. 5 and FIG. 6.

As shown in FIG. 5A, in brain slices of mice injected with the control AAV, AAV-GFAP-mCherry, the cells labeled red fluorescence exhibit typical astrocyte morphology, and there is almost no cell co-labeled with mCherry and NeuN. In the test groups injected with AAV (AAV-GFAP-mCherry+AAV-GFAP-positive regulatory gene), where the positive regulatory gene is DPYSL2, BAF45b, SCF, HuB, HuC, HuD, CYP1B1, or BTRC, there are different proportions of cells co-labeled with mCherry and NeuN (from FIGS. 5B to 5I). These results indicate that the overexpression of genes such as DPYSL2, BAF45b, SCF, HuB, HuC, HuD, CYP1B1, or BTRC can induce astrocytes to transdifferentiate into neurons with varying degrees. As shown in FIG. 6, the genes SCF, HuB, HuC, and CYP1B1 all show excellent trans-differentiation efficiency.

### Example 4. Trans-Differentiation of Astrocytes into Dopamine Neurons

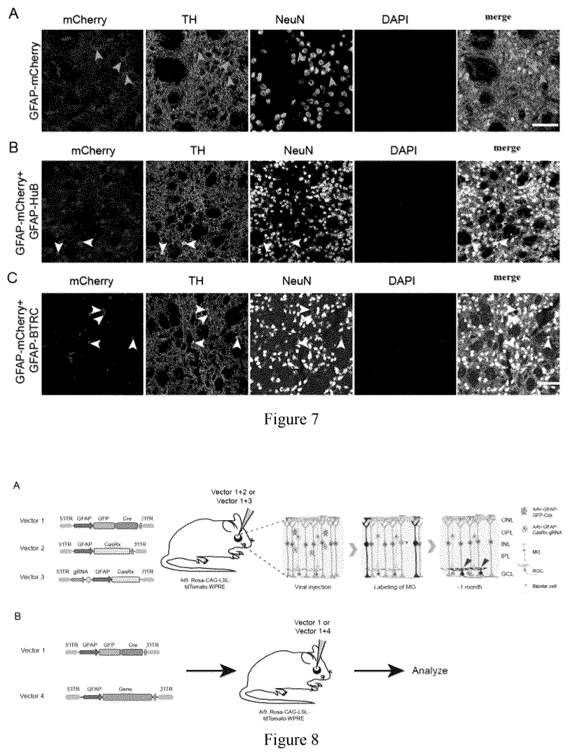

In order to further study whether the overexpression of positive regulatory genes and the inhibition of negative regulatory genes can promote the trans-differentiation of astrocytes into dopamine neurons, we inject AAVs from either the control group or the test group into the striatum of mice by reference to the operations from Example 1 to Example 3, then stain with the dopamine neuron-specific protein marker TH. The results are shown in FIG. 7. No TH-positive cells are observed in the control group (FIG. 7A). In the groups of the overexpression of HuB and BTRC, TH-positive dopamine neurons co-labeled with the mCherry signal are identified. These indicate that the overexpression of HuB and BTRC can induce the trans-differentiation of astrocytes into dopamine neurons.

### Example 5. Trans-Differentiation of Müller Glial Cells into Photoreceptor Cells

In the retina, Müller glial cells perform functions similar to glial cells, and the retina contains a variety of neurons with specific functions, such as photoreceptor cells and bipolar cells. In order to explore whether each gene can transdifferentiate Müller glial cells into photoreceptor cells in the retinal system, we use the GFAP-EGFP-2A-Cre system in combination with Ai9 mice to achieve specific labeling of Müller glial cells. The schematic diagram of the operation is shown in FIG. 8. By reference to the operations from Examples 1 to 3, a mixture of AAV-GFAP-CasRx-gRNA (or the control vector AAV-GFAP-CasRx) and AAV-GFAP-EGFP-2A-Cre is subretinally injected into 6-10-week-old Ai9 (Rosa26-CAG-LSL-tdTomato-WPRE) mice. AAV-GFAP-ECFP-2A-Cre is used to specifically label neurons (retinal ganglion cells or photoreceptor cells) transformed from Müller glial cells or glial cells. 1-3 months after injection, the retinas are taken for sectioning and analysis to study whether the Müller glia cells can transdifferentiate into photoreceptor cells in the retinal system through the inhibition of the corresponding negative regulatory genes or the up-regulation of the corresponding positive regulatory genes.

GFAP-EGFP-2A-Cre-GFAP-CasRx is the control group. GFAP-EGFP-2A-Cre-GFAP-CasRx-gRNA is the test group. The gRNA target Sin3a, HDAC2, BAF53a, G9a, HuR, BrG1, EZH2, CDYL, or KDM1A.

GFAP-EGFP-2A-Cre is the control group. GFAP-EGFP-2A-Cre+GFAP-positive regulatory gene is the test group. The positive regulatory gene is the gene of DPYSL2, BAF45b, SCF, CYP1B1, or BTRC.

Subretinal injection of AAV from different groups is performed in NMDA-modeled Ai9 mice, following the analysis 1-3 months after injection.

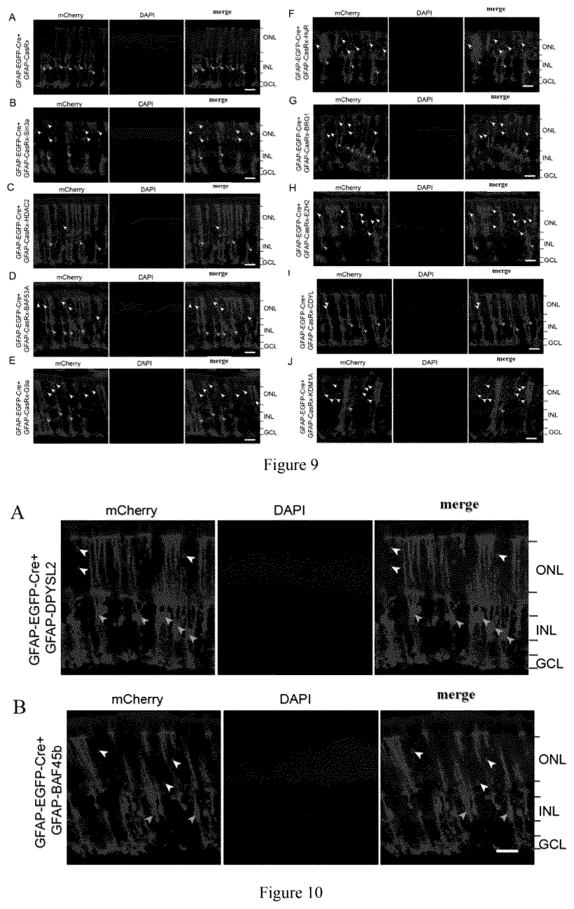

The effects of negative regulatory genes on trans-differentiation are shown in FIG. 9. In FIG. 9A, in the group of GFAP-EGFP-2A-Cre, Müller glial cells exhibited typical morphology with the cell body located in the inner nuclear layer (INL) and processes distributed in the outer granular layer (ONL) and retinal ganglion cell layer (GCL). No red signal-labeled photoreceptor cells are observed in the ONL, indicating that no Müller glia cell trans-differentiation into photoreceptor cells occurred in the control group. As shown from FIG. 9B to FIG. 9J, a large number of photoreceptor cells labeled with red fluorescence are found in the test groups of knocking down the negative regulatory gene of Sin3a, BAF53a, G9a, HuR, BrG1, EZH2, or KDM1A. These results indicate that Müller glial cells can be transdifferentiated into photoreceptor cells by knocking down the gene Sin3a, HDAC2, BAF53a, G9a, HuR, BrG1, EZH2, CDYL, or KDM1A.

The impact of positive regulatory genes on trans-differentiation is shown in FIG. 10. Red fluorescence-labeled photoreceptor cells are found in the ONL layer of the group BAF45b and DPYSL2, indicating that overexpression of DPYSL2 and BAF45b can transdifferentiate Müller glial cells into photoreceptor cells.

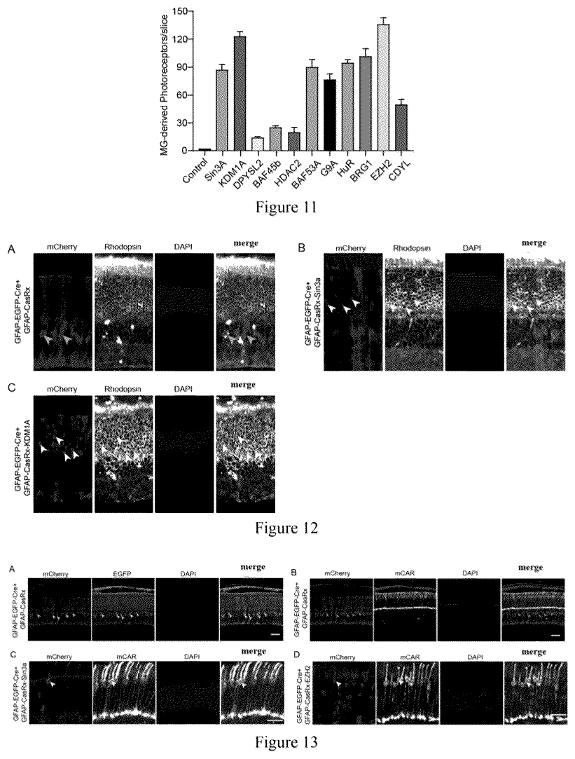

FIG. 11 shows the statistical data of Müller glial cells reprogrammed into photoreceptor cells through CasRx-mediated knockdown of negative regulatory genes or overexpression of positive regulatory genes in the retina. It shows excellent ability to regenerate photoreceptor cell when inhibiting Sin3a, KDM1A, BAF53a, G9a, HuR, BrG1, EZH2, or CDYL. Inhibiting HDAC2 also has the ability to regenerate photoreceptor cell. The overexpression of DPYSL2 and BAF45b also has the ability to regenerate photoreceptor cell.

Photoreceptor cells are divided into cones and rods. We selected the Sin3a and KDM1A groups staining with rod cell-specific protein markers. As shown in FIG. 12, it is found that most of the photoreceptor cells transdifferentiated from Müller glial cells in the ONL layer are rod cells.

We stain some groups with the cone-specific protein marker m-CAR, and perform imaging analysis under a confocal microscope. As shown in FIG. 13, cone cells are found.

### Example 6. Trans-Differentiation of Müller Glia Cells into Retinal Ganglion Cells

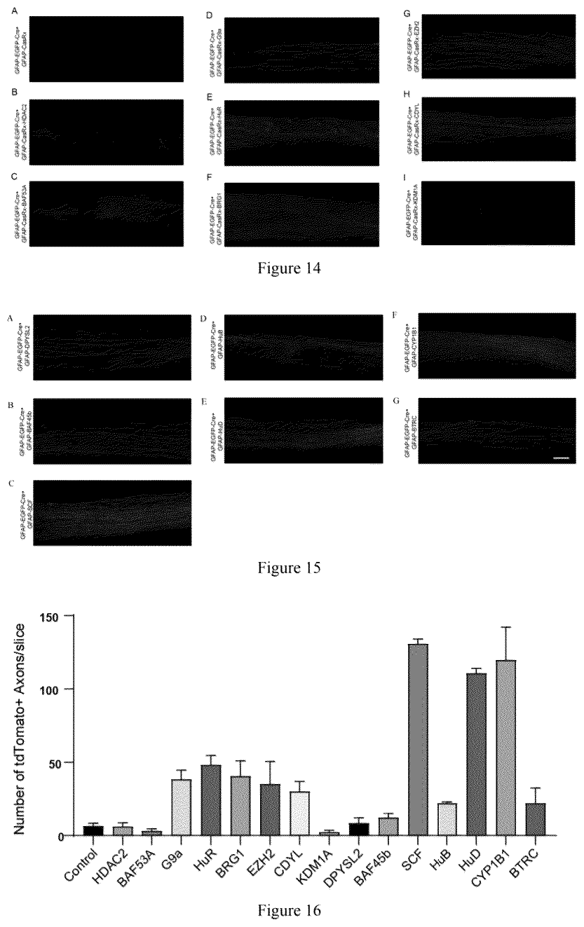

In order to study whether Müller glial cells can be transdifferentiated into retinal ganglion cells (RGC) through knocking down RCOR1, Sin3a, HDAC2, KDM1A, PHF21A, BAF53a, G9a, USP14, HuR, BrG1, EZH2, CDYL, HMG20B or overexpressing DPYSL2, BAF53b, BAF45b, SCF, HuB, HuC, HuD, CYP1B1, or BTRC, different AAV combinations are injected subretinally into NMDA-modeled 6-8 week Ai9 (Rosa26-CAG-LSL-tdTomato-WPRE) mice. In the groups of knocking down gene expression, a mixture of AAV-GFAP-CasRx-gRNA (or the control vector AAV-GFAP-CasRx) and AAV-GFAP-EGFP-2A-Cre is injected. AAV-GFAP-EGFP-2A-Cre is used to specifically label Müller glial cells or the neurons (retinal ganglion cells) transformed from glial cells. In the overexpression groups, AAV-GFAP-EGFP-2A-Cre is injected as the control group, and AAV-GFAP-EGFP-2A-Cre+AAV-GFAP-positive regulatory genes are injected (the positive regulatory gene is DPYSL2, BAF45b, SCF, HuB, HuC, HuD, CYP1B1, or BTRC). Since the pseudopodia of MO Her glial cells in the GCL layer significantly interfere with the observation of transdifferentiated RGC cells, and each RGC cell sends an axon to the optic nerve, we adopted an optic nerve imaging analysis method. The optic nerves are harvested for analysis 1-3 months after injection. The results are shown in FIG. 14 and FIG. 15. As shown in FIG. 14A, in the control group, very few optic nerves are found, and an average of less than 5 axons labeled by red fluorescent signal are observed in each optic nerve. As shown from FIG. 14B to FIG. 141, in the HuR knockdown group, a large number of axons labeled with red fluorescent signals are detected, while almost no obvious axon signals labeled with red fluorescent signals are observed in the KDM1A, Sin3a, and HDAC2 group. As shown in FIG. 15, in the groups overexpressing SCF, HuD, and CYP1B1, a large number of axons labeled with red fluorescent signals are observed, and there are the most numbers of axons labeled with red fluorescent signals exist in the SCF group. These results indicate that knocking down the expression of HuR or overexpressing SCF, HuD, and CYP1B1 can promote the trans-differentiation of Müller glial cells to RGCs, while the gene of KDM1A, Sin3a, HDAC2 don't exhibit the differentiated function of Müller glial cells to RGC.

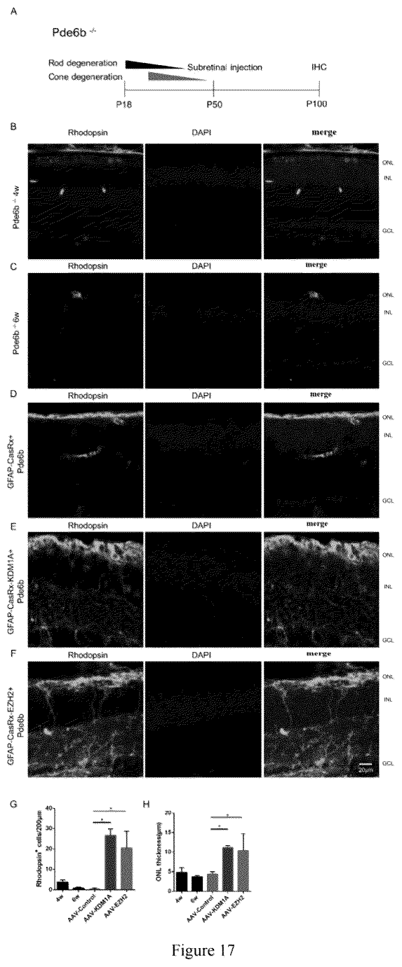

A statistical analysis is conducted for the number of Müller glial cells reprogrammed into retinal ganglion cells in the retina through knocking down the expression of KDM1A, HDAC2, BAF53a, G9a, HuR, BrG1, EZH2, and CDYL, or overexpressing DPYSL2, BAF45b, SCF, HuB, HuD, CYP1B1, and BTRC. FIG. 16 shows a statistical graph of the number of tdTomato-positive axons in the optic nerve.

A greater number of retinal ganglion cells are observed in the group of SCF, HuD, and CYP1B1.

### Example 7: Photoreceptor Cell Regeneration Found in RP Disease Model Mice

Methods: The Pde6b gene is a pathogenic gene related to retinitis pigmentosa (RP). Defects in the Pde6b gene can lead to the death of photoreceptor cells. Currently, mice with Pde6b gene defects have been proven to be animal models of diseases with RP genetic characteristics. In this patent, a Pde6b gene knockout mouse model is constructed based on CRISPR-Cas9 editing. Through immunohistochemical analysis of Pde6b gene knockout mice, it is demonstrated that many rod cells die 4 weeks after birth, and all rod cells completely die of 6 weeks afterbirth (FIG. 17A-C). At 7 weeks of age in this disease model mouse, subretinal injections of AAVs targeting the trans-differentiation targets KDM1A or EZH2 (AAV-U6-gRNA(KDM1A)-GFAP-CasRx and AAV-U6-gRNA(EZH2)-GFAP-CasRx) or the AAV overexpressing Pde6b(AAV-CBH-Pde6b) are administered simultaneously. The control group is simultaneously injected with control AAV (AAV-GFAP-CasRx) and AAV overexpressing Pde6b (AAV-CBH-Pde6b). The injection volume of the AAVs targeting the trans-differentiation targets is 1×10¹⁰ vg, The injection volume of the AAV overexpressing Pde6b is 5×10⁹ vg, the total volume of injected AAV is 1 μl. Retinal tissue is taken for analysis two months after injection, no photoreceptor cells are found in the outer granular layer of the retina in the mice injected with control group AAV (AAV-GFAP-CasRx+AAV-CBH-Pde6b). Staining with the rod cell-specific marker rhodopsin, no rhodopsin-positive cells are found (FIG. 17)). In the groups injected with AAVs targeting the trans-differentiation targets KDM1A or EZH2 (AAV-U6-gRNA(KDM1A)-GFAP-CasRx and AAV-U6-gRNA(EZH2)-GFAP-CasRx), a layer of cells is found in the outer granular layer of the mouse retina. Immunofluorescence staining shows that these cells are rhodopsin-positive photoreceptor cells (FIG. 17E-F). Statistical analysis of each group shows that in the group with KDM1A and EZH2 knockdown, more than 20 rhodopsin-positive photoreceptor cells are generated in 200 μm (FIG. 170). Analyzing the thickness of the outer granular layer, the thickness significantly increases in the outer granular layer of the groups knocking down KDM1A and EZH2. (FIG. 17H).

In the above-mentioned examples (such as Example 3 and Example 4), the applicant described the results of injecting the negative regulator or the positive regulator of the present application into the striatum in detail. According to the technical solution disclosed in this application and the conventional knowledge in the field, those skilled in the art can choose to perform targeted injections in other regions of the brain, such as the ventral tegmental area of the midbrain, spinal cord, hypothalamus, dorsal midbrain, cerebral cortex, hippocampus, cerebellum, or substantia nigra, particularly in the substantia nigra, which would yield the same or similar technical effects.

In the above-mentioned examples (for example, Example 5), the applicant described the results of subretinal injection of the negative regulator or positive regulator of the present application in detail. According to the technical solution disclosed in this application and the conventional knowledge in the field, those skilled in the art can select other locations in the eye for targeted injection, such as the vitreous cavity, which would yield the same or similar technical effects.

It should be understood that the above embodiments are merely illustrative and not specifically limiting. For example, in the above embodiments, the applicant described the results of negative regulation of the RCOR1 gene in detail. According to the technical solution disclosed in this application and the conventional knowledge in the field, those skilled in the art can choose to regulate genes similar to RCOR1 (such as RCOR2 or RCOR3) to achieve the same or similar technical effects. The applicant has described the results of negatively regulating the Sin3a gene in detail, those skilled in the art can choose to regulate genes similar to Sin3a (such as Sin3b) according to the technical solution disclosed in this application and the conventional knowledge in the field and achieve the same or similar technical effects. The applicant has described the results of negatively regulating the HDAC2 gene in detail, those skilled in the art can choose to regulate genes similar to HDAC2 (such as HDAC1) according to the technical solution disclosed in this application and the conventional knowledge in the field, and achieve the same or similar technical effects.

All documents mentioned in this disclosure are hereby cited for reference in this application as if each document is individually cited for reference. In addition, it should be understood that, after reading the teachings provided in this disclosure, those skilled in the art can make various changes or modifications to this disclosure, and these equivalent forms also fall the scope of the claims in this application.

The following is the sequence information of positive regulatory genes and negative regulatory genes involved in this application:

Human RCOR1 protein sequence
(SEQ ID NO: 1)
MPAMVEKGPEVSGKRRGRNNAAASASAAAASAAASAACASPAATAASG
AAASSASAAAASAAAAPNNGQNKSLAAAAPNGNSSSNSWEEGSSGSSSDEEH
GGGGMRVGPQYQAVVPDFDPAKLARRSQERDNLGMLVWSPNQNLSEAKLDE
YIAIAKEKHGYNMEQALGMLFWHKHNIEKSLADLPNFTPFPDEWTVEDKVLF
EQAFSFHGKTFHRIQQMLPDKSIASLVKFYYSWKKTRTKTSVMDRHARKQKR
EREESEDELEEANGNNPIDIEVDQNKESKKEVPPTETVPQVKKEKHSTQAKNR
AKRKPPKGMFLSQEDVEAVSANATAATTVLRQLDMELVSVKRQIQNIKQTNS
ALKEKLDGGIEPYRLPEVIQKCNARWTTEEQLLAVQAIRKYGRDFQAISDVIG
NKSVVQVKNFFVNYRRRFNIDEVLQEWEAEHGKEETNGPSNQKPVKSPDNSI
KMPEEEDEAPVLDVRYASAS
Human RCOR1 coding sequence
(SEQ ID NO: 2)
atgccggccatggtggagaagggccccgaggtctcagggaagcggagagggaggaacaacgcggccgcctc
cgcctccgccgccgccgcctccgccgccgcctcggccgcctgcgcctcgccagccgccactgccgcctcgggcgccg
ccgcctcctcagcctcggccgccgccgcctcagccgccgccgcccccaataatggccagaataaaagtttggcggcggc
ggcgcccaatggcaacagcagcagcaactcctgggaggaaggcagctcgggctcgtccagcgacgaggagcacggt
ggcggtggcatgagggtcggaccccagtaccaggcggtggtgcccgacttcgaccccgccaaactggcaagacgcagt
caagaacgggacaatcttggcatgttggtctggtcacccaatcaaaatctgtcagaagcaaagttggatgaatacattgccat
tgccaaagaaaagcatgggtacaacatggaacaggctcttgggatgctcttctggcataaacataatatcgaaaagtcattg
gctgatttgcccaactttacccctttcccagatgagtggactgtggaagataaagtcttatttgagcaagcctttagttttcatgg
gaaaacttttcatagaatccaacaaatgcttccagataaatctatagcaagtctggtgaaattttactattcttggaagaagacg
aggactaaaactagtgtgatggatcgccatgcccggaaacaaaaacgggagcgggaggagagcgaggatgaactgga
agaggcaaatggaaacaatcccattgacattgaggttgatcaaaacaaggaaagcaaaaaggaggttccccctactgaga
cagttcctcaggtcaaaaaagaaaaacatagcacacaagctaaaaatagagcaaaaaggaaacctccaaaaggaatgttt
ctttctcaagaagatgtggaggctgtttctgccaatgccactgctgctaccacggtgctgagacaactagacatggaattggt
ttcagtcaaacgacagatccagaatattaaacagacaaacagtgctctcaaagaaaaacttgatggtggaatagaaccatat
cgacttccagaggtcattcagaaatgtaatgcacgttggactacagaagagcagcttctcgccgtacaagccatcaggaaa
tatggccgagattttcaggcaatctcagacgtgattgggaacaaatcagtggtacaagtgaaaaacttttttgtaaattatcga
cgccgcttcaacatagatgaagttttacaagaatgggaggcagaacatggtaaagaagagaccaatgggcccagtaacca
gaagcctgtgaagtccccagataattccattaagatgcccgaagaggaagacgaggctcctgttctggatgtcagatatgc
atctgcctcctga
Human RCOR2 protein sequence
(SEQ ID NO: 3)
MPSVMEKPSAGSGILSRSRAKTVPNGGQPHSEDDSSEEEHSHDSMIRVGT
NYQAVIPECKPESPARYSNKELKGMLVWSPNHCVSDAKLDKYIAMAKEKHG
YNTEQALGMLLWHKHDVEKSLADLANFTPFPDEWTVEDKVLFEQAFGFHGK
CFQRIQQMLPDKLIPSLVKYYYSWKKTRSRTSVMDRQARRLGGRKDKEDSDE
LEEGRGGVSEGEPDPADPKREPLPSRPLNARPGPGKKEVQVSQYRHHPLRTRR
RPPKGMYLSPEGLTAVSGSPDLANLTLRGLDSQLISLKRQYQSMKQTNSSLRQ
ALEGGIDPLRPPEANTKFNSRWTTDEQLLAVQAIRRYGKDFGAIAEVIGNKTLT
QVKTFFVSYRRRFNLEEVLQEWEAEQDGAPGAPVPMEEARRGAPLPAPALEE
DDEVQITSVSTSVPRSVPPAPPPPPPPTSLSQPPPLLRPPLPTAPTLLRQPPPLQQ
GRFLQPRLAPNQPPPPLIRPALAAPRHSARPGPQPPPTLTGTPLEPPAPSL
Human RCOR2 coding sequence
(SEQ ID NO: 4)
atgccctcagtgatggagaagccgagcgcgggctctgggatcctgtcccgtagccgggccaagacggtgcccaa
cggcggacagccccactcggaggatgacagcagcgaggaggagcactcgcacgacagcatgatccgcgttggaacca
attaccaggccgtaattccggagtgcaagcctgagagccccgcacgctacagcaacaaggagctgaaggggatgctggt
gtggtcacccaaccactgtgtgtcagatgccaagcttgacaagtacattgcgatggccaaggagaagcatggctacaacat
tgagcaggcgctgggcatgcttctgtggcataagcacgatgtggagaagtcgctggccgacctggccaacttcaccccatt
ccctgacgagtggacagtagaggacaaggtgctgtttgaacaggcctttggcttccatggcaaatgcttccagcggatcca
gcagatgctgcctgacaagttgattcccagcctggtgaaatactactactcttggaagaagacccgcagccgaactagtgtg
atggacagacaggcccggcggctggggggccgcaaggacaaagaagacagtgatgagctcgaagagggtcgaggag
gcgtgagtgagggagagcccgatcctgcagatcccaagagagagcctctaccctctcggcccctgaatgcacgcccagg
ccctgggaaaaaggaggtccaggtgtctcagtaccgccaccatcccttgcgaacccggcgtcgcccacccaagggcatg
tacctgagccctgaaggcctcacggcagtgtcaggaagcccggaccttgccaacctcacgctccgaggtcttgacttctca
gctcatctccctcaagcgccaggtacagagcatgaagcagacgaacagcagcctgcgccaagccctggagggcggtatt
gatccactacgccccccggaggccaacaccaagttcaactcccgctggaccacagatgagcagcttttggctgttcaagc
catccgtaggtatggcaaagactttggggctattgcagaggtgattgggaacaagactctgacccaggtgaagactttctttg
tgagctaccggcgccgcttcaatctggaggaggtgctgcaggaatgggaggctgagcaggatggggcccctggagccc
cagtccccatggaggaggctaggagaggggctccattgccagccccagccctagaggaagatgatgaggtccagattac
atcggtctccacgtccgtgccccgatcagtgccccctgcgccaccaccccctccacctcccacctcgctgtcccagccacc
cccgctgctgaggccacctttgcccacggctcccactctgctccgacagccacccccactgcagcagggccgcttcctcc
agccccggctggcccccaaccagcccccaccgcctctcatccgccccgctctggctgccccccgccacagcgcccgcc
ctggccctcagcccccacccaccctgattggaacccctctggagcccccagcaccctcactctga
Human RCOR3 protein sequence
(SEQ ID NO: 5)
MPGMMEKGPELLGKNRSANGSAKSPAGGGGSGASSTNGGLHYSEPESG
CSSDDEHDVGMRVGAEYQARIPEFDPGATKYTDKDNGGMLVWSPYHSIPDA
KLDEYIAIAKEKHGYNVEQALGMLFWHKHNIEKSLADLPNFTPFPDEWTVED
KVLFEQAFSFHGKSFHRIQQMLPDKTIASLVKYYYSWKKTRSRTSLMDRQAR
KLANRHNQGDSDDDVEETHPMDGNDSDYDPKKEAKKEGNTEQPVQTSKIGL
GRREYQSLQHRHHSQRSKCRPPKGMYLTQEDWAVSCSPNAANTILRQLDME
LISLKRQVQNAKQVNSALKQKMEGGIEEFKPPESNQKINARWTTEEQLLAVQ
GVRKYGKDFQAIADVIGNKTVGQVKNFFVNYRRRFNLEEVLQEWEAEQGTQ
ASNGDASTLGEETKSASNVPSGKSTDEEEEAQTPQAPRTLGPSPPAPSSTPTPT
APIATLNQPPPLLRPTLPAAPALHRQPPPLQQQARFIQPRPTLNQPPPPLIRPANS
MPPRLNPRPVLSTVGGQQPPSLTGIQTDSQSSLH
Human RCOR3 coding sequence
(SEQ ID NO: 6)
atgcccggcatgatggagaaagggcccgagttactggggaagaaccgatcggccaacggcagcgccaagagc
ccggcaggcggcggcggcagcggcgcctcgtccaccaacggcgggctgcactactcagagcccgagagcggctgca
gcagcgacgacgagcacgatgttgggatgagagtcggagccgaataccaagctcggatccctgaatttgatccaggtgct
acaaagtacacagataaagacaatggagggatgcttgtatggtctccatatcacagtatcccagatgccaaattggatgaat
acattgcaattgcaaaggaaaagcatggctacaatgtggaacaggcacttggcatgttgttctggcataaacataacattga
gaagtcccttgctgatctccctaatttcactccctttccggatgagtggacagtggaagataaagtcctatttgaacaagccttt
agttttcatggaaagagctttcacaggattcagcaaatgcttccagataagacaattgcaagccttgtaaaatattactattcttg
gaaaaaaactcgctctaggacaagtttgatggatcgccaggctcgtaaactagctaatagacataatcagggtgacagtgat
gatgatgtagaagaaacacatccaatggatgggaatgatagtgattatgatcccaaaaaagaagccaaaaaagagggtaat
actgaacaacctgtccaaactagcaagattggacttggaagaagagagtatcagagtttacaacatcgccatcattctcagc
gttctaagtgccgtccacctaagggcatgtatttaacccaggaagatgtggtagcagtttcctgtagtcccaatgcagccaac
accatcctgaggcaactggacatggagttgatctctctaaaacgtcaggttcagaatgctaagcaagtaaacagtgcactta
aacagaaaatggaaggtggaattgaagaattcaaacctcctgagtcaaatcagaaaattaatgcccgttggaccacagagg
agcagcttctagcagtgcaaggtgtccgcaaatatggtaaagattttcaagctattgcagatgtaattggcaacaagactgtt
ggccaagtgaagaacttctttgtaaactacaggcgtcggtttaacttagaggaggtattgcaggagtgggaagcagaacaa
ggaacccaggcttctaatggtgatgcttctactttaggggaggagacaaaaagtgcttctaatgtgccatcagggaagagca
ctgatgaagaagaggaggcacagaccccacaggctcctcggacactgggtccatcacctcctgccccatcatccactcca
acaccaacagcccctattgccactctgaaccagcctccaccacttcttcgtccaacactgcctgctgccccggctcttcacc
ggcagcctcctccactccagcagcaggctcggttcatccagccccggccaactttaaatcagcctccaccacctcttattcg
ccctgctaattccatgccaccccgtctaaacccaagaccggtgttgtccacggttggtggtcaacagccaccatcacttattg
gaattcagacagattcacagtcctcactgcactaa
human Sin3a protein sequence
(SEQ ID NO: 7)
MKRRLDDQESPYYAAQQRRIPGSTEAFPHQHRYLAPAPPVYEAYSETMQ
SATGIQYSYTPSYQYSAMPQSSGSHGPAIAAYHSSHHHPTAVQPHGGQYYQSH
AHPAPPVAPVQGQQQFQRLKVEDALSYLDQVKLQFGSQPQYYNDFLDIMKEF
KSQSIDTPGVISRVSQLFKGHPDLIMGFNTFLPPGYKIEVQTNDMVNVTTPGQ
VHQIPTHGIQPQPQPPPQHPSQPSAQSAPAPAQPAPQPPPAKVSKPSQLQAHTPA
SQQTPPLPPYASPRSPPVQPHTPVTISLGTAPSLQNNQPVEFNHAINYVNKIKNR
FQGQPDIYKAFLEILHTYQKEQRNAKEAGGNYTPALTEQEVYAQVARLFKNQ
EDLLSEFGQFLPDANSSYLLSKTTAEKYDSYRNDHGGTVKKPQLNNKPQRPS
QNGCQIRRHPTGTTPPVKKKPKLLNLKDSSMADASKHGGGTESLFFDKVRKA
LRSAEAYENFLRCLVIFNQEVISRAELVQLYSPFLGKFPELFNWFKNFLGYKES
VHLETYPKERATEGIAMEIDYASCKRLGSSYRALPKSYQQPKCTGRTPLCKEV
LNDTWVSFPSWSEDSTFYSSKKTQYEEHIYRCEDERFELDVVLETNLATIRVLE
AIQKKLSRLSAEEQAKFRLDNTLGGTSEYIHRKALQRIYADKAADIIDGLRKN
PSIAVPIVLKRLKMKEEEWREAQRGFNKVWREQNEKYYLKSLDHQGINFKQN
DTKVLRSKSLLNEIESIYDERQEQATEENAGVPVGPHLSLAYEDKQILEDAAA
LIIHHVKRQTGIQKEDKYKIKQIMHHFIPDLLFAQRGDLSDVEEEEEEEMDVDE
ATGAVKKHNGVGGSPPKSKLLFSNTAAQKLRGMDEVYNLFYVNNNWYTFMR
LHQILCLRLLRICSQAERQIEEENREREWEREYLGIKRDKSDSPAIQLRLKEPM
DVDVEDYYPAFLDMVRSLLDGNIDSSQYEDSLREMFTIHAYIAFTMDKLIQSI
VRQLQHIVSDEICVQVTDLYLAENNNGATGGQLNTQNSRSLLESTYQRKAEQ
LMSDENCFKLMFIQSQGQVQLTIELLDTEEENSDDPVEAERWSDYVERYMNS
DTTSPELREHLAQKPVFLPRNLRRIRKCQRGREQQEKEGKEGNSKKTMENVD
SLDKLECRFKLNSYKMVYYIKSEDYMYRRTALLRAHQSHERYSKRLHQRFQA
WVDKWTKEHVPREMAAETSKWLMGEGLEGLYPCTTTCDTETLHFVSINKYR
VKYGTVFKAP
Human Sin3a coding sequence
(SEQ ID NO: 8)
atgaagcggcgtttggatgaccaggagtcaccggtgtatgcagcccagcagcgtcggatccctggcagcacaga
ggcttttcctcaccagcaccgggtgcttgcccctgcccctcctgtgtatgaagcagtgtctgagaccatgcagtcagctacg
ggaattcagtactctgtaacacccagctaccaggtttcagccatgccacagagctccggcagtcatgggcccgctatagca
gcagttcatagcagccatcatcacccaacagcggtgcagccccacggaggccaggtggtccagagtcatgctcatccag
ccccaccagttgcaccagtgcagggacagcagcaatttcagaggctgaaggtggaggatgcgctatcttatcttgaccag
gtgaagctgcagtttggtagtcagcctcaggtctacaatgatttccttgacatcatgaaggaatttaaatctcagagcatcgac
accccaggagtgattagtcgtgtgtcccagctattcaaaggccaccccgatctgataatgggattcaacaccttcttgccccc
tggctacaaaattgaggtgcaaaccaatgacatggtgaatgtgacaactcctggccaggttcatcagattcccacccatggc
atccagccacagcctcaaccaccaccccaacatccttcccagccttcagcccagtcagccccagctcctgcccagccagc
tcctcagcccccacctgccaaagtcagcaagccctcccaactgcaagcacatactccggccagtcagcagactcccccac
ttccaccgtatgcatccccacgttctccgccggtccagcctcacacaccagtgacaatctcgttgggaacggccccatcctt
gcagaacaatcaacctgtggagtttaatcatgccatcaactatgttaataagatcaagaacagatttcagggccaaccagac
atctacaaagcattcctggagattttgcacacatatcagaaagagcagagaaatgccaaggaagctggaggaaactacact
ccagcattgacagagcaggaggtgtatgcccaggttgctcgtctctttaaaaaccaggaagatttgttgtcagagtttggaca
attcctaccagatgccaacagctccgtgcttttaagcaaaacaactgctgagaaggttgattctgtgagaaatgatcatggag
gcactgtcaagaagccccaactgaacaacaagccgcagaggcccagccagaatggctgccagatccgcagacatccta
caggaaccacccctccagttaagaagaaacccaaactgctcaatctgaaggattcttctatggcagatgccagcaaacatg
gtggtggaacagaatcgttattttttgataaggtccgaaaggctcttcggagtgcagaagcctacgaaaatttcctacgctgtc
ttgttatttttaaccaggaggtgatctctcgtgctgagcttgtgcaactagtctctcctttcctggggaaattccctgagttgtttaa
ttggtttaaaaactttctgggctataaggagtctgtacatctggaaacttatccaaaggagcgagccacagagggcattgcta
tggagatagattatgcttcttgtaaacgattgggctccagctatcgagccttaccaaagagttaccagcagcccaagtgtaca
ggacggactcctctctgtaaagaggttttaaatgatacctgggtttccttcccttcgtggtctgaggactctacctttgtgagttc
caagaagactcaatatgaagaacatatttatcgttgtgaagatgaacgctttgagcttgatgtagttttagagaccaatctggc
aacaatccgggttctggaagcaatacagaagaagctttcccgcttgtctgctgaagaacaagccaaatttcgcttggacaac
acccttgggggcacatcagaagtcatccatagaaaagcactccagaggatatatgctgataaagcagctgacatcatttgat
ggtctgagaaagaatccctccattgctgttccaattgtccttaaaaggttgaagatgaaagaggaagaatggcgagaagctc
agagaggctttaacaaagtatggcgagaacaaaatgagaaatactacttgaagtctctggaccaccaggggatcaacttta
aacagaatgacaccaaggtcctgaggtctaagagcttactcaatgagattgagagtatctatgatgagaggcaagagcagg
ctacggaggagaatgctggtgtacctgttggcccacacctctcacttgcgtatgaagacaaacaaatactggaagatgctgc
tgctctgattatccaccatgtgaagaggcagacaggcattcagaaggaggacaaatataagataaaacaaatcatgcatcat
tttattccagatttgctctttgcccaaagaggtgatctctcagatgtggaggaagaggaagaagaagagatggatgtagatga
agccacaggggcagttaagaagcacaatggtgttgggggcagtccccctaagtccaagttactgtttagtaacacagcagc
tcaaaaattaagaggaatggatgaagtatacaacctcttctatgtcaacaacaactggtatatttttatgcgactgcaccagatt
ctctgcctgaggctgctacggatttgttcccaagccgaacggcaaattgaagaagaaaaccgagagagagaatgggaac
gggaagtgctgggcataaagcgagacaagagtgacagccctgccattcagctacgtctcaaagaacctatggatgttgat
gtagaagattattacccagctttcctggacatggtgcggagcctgctggatggcaacatagactcatcacagtatgaagattc
actgagagagatgttcaccattcatgcctacattgcctttaccatggacaaactgatccagagcattgtcagacagctgcagc
atatcgtgagtgatgagatctgtgtgcaggtgactgacctttacctggcagaaaataataatggggccaccggaggccagc
tgaacacacagaactcaaggagcctcctggagtcaacgtatcagcggaaagctgagcagctaatgtcagatgagaattgc
tttaagcttatgtttattcagagccaaggccaggtccagctgactattgagcttctggacacagaagaggagaattcggatga
ccctgtggaagcagagcgctggtcagactacgtggagcgatacatgaattcagatactacctcgcctgagcttcgtgaaca
tctagcacagaaaccagtatttctccccaggaatctacggcggatccggaagtgtcaacgtggtcgagagcagcaggaaa
aggaagggaaggaaggaaacagcaagaagaccatggagaatgtggatagtctggataagctggagtgtagattcaagct
gaattcctacaagatggtgtatgtgatcaaatcagaggactatatgtatcggaggaccgccctgctccgggctcatcagtcc
catgagcgtgtaagcaagcgtctacatcagagattccaggcctgggtagataaatggaccaaggagcatgtgccccgtga
aatggcagcagagaccagcaagtggctcatgggtgaggggctggagggcctggtgccctgtaccaccacctgtgataca
gagaccctgcattttgtgagcattaacaagtatcgtgtcaaatacggcacagtattcaaagccccttaa
human Sin3b protein sequence
(SEQ ID NO: 9)
MAHAGGGSGGSGAGGPAGRGLSGARWGRSGSAGHEKLPVHVEDALTY
LDQVKIRFGSDPATYNGFLETMKEFKSQSIDTPGVIRRVSQLFHEHPDLIVGFNA
FLPLGYRIDIPKNGKLNIQSPLTSQENSHNHGDGAEDFKQQVPYKEDKPQVPL
ESDSVEFNNAISYVNKIKTRFLDHPEIYRSFLEILHTYQKEQLNTRGRPFRGMS
EEEVFTEVANLFRGQEDLLSEFGQFLPEAKRSLFTGNGPCEMHSVQKNEHDKT
PEHSRKRSRPSLLRPVSAPAKKKMKLRGTKDLSIAAVGKYGTLQEFSFFDKVR
RVLKSQEVYENFLRCIALFNQELVSGSELLQLVSPFLGKFPELFAQFKSFLGVK
ELSFAPPMSDRSGDGISREIDYASCKRIGSSYRALPKTYQQPKCSGRTAICKELD
HWTLLQGSWTDDYCMSKFKNTCWTPGYSAGVLNDTWVSFPSWSEDSTFVSS
KKTPYEEQLHRCEDERFELDVVLETNLATIRVLESVQKKLSRMAPEDQEKFRL
DDSLGGTSEVIQRRAIYRIYGDKAPEIIESLKKNPVTAVPVVLKRLKAKEEEWR
EAQQGFNKIWREQYEKAYLKSLDHQAVNFKQNDTKALRSKSLLNEIESVYDE
HQEQHSEGRSAPSSEPHLIFVYEDRQTLEDAAALISYYVKRQPAIQKEDQGTIH
QLLHQFVPSLFFSQQLDLGASEESADEDRDSPQGQTTDPSERKKPAPGPHSSPP
EEKGAFGDAPATEQPPLPPPAPHKPLDDVYSLFFANNNWYFFLRLHQTLCSRL
LKIYRQAQKQLLEYRTEKEREKLLCEGRREKGSDPAMELRLKQPSEVELEEY
YPAFLDMVRSLLEGSIDPTQYEDTLREMFTIHAYVGFTMDKLVQNIARQLHHL
VSDDVCLKVVELYLNEKKRGAAGGNLSSRCVRAARETSYQWKAERCMADE
NCFKVMFLQRKGQVIMTIELLDTEEAQTEDPVEVQHLARYVEQYVGTEGASS
SPTEGFLLKPVFLQRNLKKFRRRWQSEQARALRGEARSSWKRLVGVESACDV
DCRFKLSTHKMVFIVNSEDYMYRRGTLCRAKQVQPLVLLRHHQHFEEWHSR
WLEDNVTVEAASLVQDWLMGEEDEDMVPCKTLCETVHVHGLPVTRYRVQY
SRRPASP
Human Sin3b coding sequence
(SEQ ID NO: 10)
atggcgcacgctggcggtggcagcggtggcagcggtgccggcggccccgcgggccgggggctgagcggcgc
ccgctggggtcgctcgggctccgcaggccacgagaagctgccggtgcacgtagaagacgccctcacctatctggacca
ggtgaagatccgctttggcagcgaccctgccacctacaacggcttcctggagatcatgaaggagttcaaaagccagagca
tcgatactcctggagtcatcagacgtgtctcgcagctcttccacgagcaccctgacctcattgttggattcaacgcttttcttcc
cctcggatatagaatagacattcccaagaatggcaagttaaacatacagtcgcctctgacaagccaggagaattcgcacaa
ccacggggacggtgcagaggacttcaagcagcaggtgccgtataaagaggacaaaccccaggtgcccctggagtccga
ttccgtggaattcaacaacgccatcagctatgtgaataagattaaaacccgcttcctagaccacccagaaatctacaggtcat
tcctggagatcctgcacacgtaccagaaggagcagctgaacacgaggggccggccattccgaggcatgtctgaagagg
aggtgttcaccgaggtggccaacctcttccggggccaggaggacctgctctcagagtttggacagttcctgcccgaagcc
aagcggtctctgttcacaggaaacgggccgtgcgagatgcacagcgtgcagaagaacgagcacgacaagaccccgga
gcacagcaggaagcgctcccggccctcgctcctccgccccgtgtctgcacccgccaagaaaaaaatgaaacttcgtggta
ccaaagacctgtccatcgctgcagtggggaagtacgggactctgcaggaattttctttctttgacaaggtccgccgggtgct
gaagagccaggaggtgtatgaaaacttcctccgctgcatcgcactcttcaaccaggagctggtgtctggctctgagctcctg
cagctcgtcagcccatttctggggaaatttccagaactctttgcacagttcaagtccttcctgggggtaaaagagctgtccttc
gcgccacccatgagcgacagatccggggacgggataagccgggaaattgattatgcatcctgcaagcgcataggatcca
gctaccgggcactccccaaaacctaccagcagcccaagtgcagtgggaggacagccatctgcaaggagcttgaccattg
gacacttctccagggttcgtggacagacgattactgcatgtccaagttcaagaatacctgctggattccaggatatagtgcag
gggtactgaacgacacctgggtctccttcccttcctggtctgaggactccacgttcgtcagctccaagaagacaccgtacga
ggagcagcttcaccgctgtgaggacgagcgcttcgagttagacgttgtcctggagacgaacctggccacaatccgtgtgtt
ggaaagtgtgcagaagaagctgtctcggatggcgccggaagaccaggagaagttccggctggacgactccctgggagg
cacgtcggaggtgatccagcgccgtgccatttatcgcatctatggcgacaaggccccggagatcatcgagagcctcaaga
agaaccctgtcaccgctgtccccgttgtcctgaaaagactgaaggccaaggaagaggagtggcgggaggcccagcagg
gcttcaacaagatctggcgggagcagtatgagaaggcgtacctcaagtcccttgaccaccaggctgtgaacttcaagcag
aacgacaccaaggccctgcgctccaagagcttgctcaacgagatcgagagcgtctacgacgagcaccaggagcagcac
tcggagggccgcagtgccccctctagcgagccgcacctcatctttgtgtacgaggaccggcagatcctggaggacgcag
cagcgctcatcagctactacgtgaagcggcagccggccatccagaaggaggaccagggcaccatccaccagctgctgc
accagttcgtgcccagcctcttcttctctcagcagctggacctgggcgcctccgaggagtcagctgatgaggaccgggac
agcccccaggggcagaccacagaccccagtgagcggaagaagccggcgccaggaccccacagtagccccccagag
gagaagggggccttcggggatgccccggccactgagcagccacccctgccgcccccagccccgcacaagcccctgga
cgatgtctacagcctattttttgccaacaacaactggtacttcttcctgcgcctgcaccagaccctgtgctccaggctgctgaa
gatctaccgccaggcgcagaagcagcttctggagtatcggaccgagaaggagcgggagaagctgctgtgtgagggccg
cagggagaagggcagcgaccccgccatggagctgcggctgaagcagcccagtgaagtggagctggaggagtactacc
cggccttcctggacatggtgcggagcctgctggagggcagcatcgaccccacgcagtacgaggacaccctacgcgaga
tgttcaccatccatgcctacgtgggcttcaccatggacaagctggtgcagaacattgcgcggcagctgcaccacctcgtga
gcgatgacgtctgcctgaaggtggtggagctctacctgaacgagaagaagcggggtgccgctggtgggaacctgtcctc
ccgctgcgtccgcgctgctagggagaccagctaccagtggaaggctgagcgctgcatggccgacgagaactgcttcaag
gtgatgttcctgcagcgcaaagggcaggtgatcatgaccatcgagctcctggacaccgaggaggcccagacggaggac
cctgtggaggtccagcacctggctcggtacgtggagcagtatgtggggaccgagggcgcgtccagctcgcccactgag
ggcttcctcctgaaacctgtgttcctgcagaggaacctcaagaagttccgccgccggtggcagagcgagcaggcgcggg
ccctgcgcggtgaggccaggagctcctggaagcggctggtgggcgtggagagcgcctgcgacgtggactgccgcttca
agctcagcactcacaagatggtgttcatcgtgaactccgaggactacatgtaccgtcgcgggaccctctgccgggccaag
caggtgcagcccctggtcctgctccgccaccaccagcactttgaggagtggcacagccgctggctggaggacaatgtga
cggtggaggcggctagcctggtgcaggactggctgatgggtgaggaggacgaggacatggtaccctgcaagacgctgt
gtgagacagtgcacgtgcacggcctgcccgtgacccgctaccgcgtgcagtacagccgccgcccggcctcgccctga
human HDAC1 protein sequence
(SEQ ID NO: 11)
MAQTQGTRRKVCYYYDGDVGNYYYGQGHPMKPHRIRMTHNLLLNYG
LYRKMEIYRPHKANAEEMTKYHSDDYIKFLRSIRPDNMSEYSKQMQRFNVGE
DCPVFDGLFEFCQLSTGGSVASAVKLNKQQTDIAVNWAGGLHHAKKSEASGF
CYVNDIVLAILELLKYHQRVLYIDIDIHHGDGVEEAFYTTDRVMTVSFHKYGE
YFPGTGDLRDIGAGKGKYYAVNYPLRDGIDDESYEAIFKPVMSKVMEMFQPS
AVVLQCGSDSLSGDRLGCFNLTIKGHAKCVEFVKSFNLPMLMLGGGGYTIRN
YARCWTYETAVALDTEIPNELPYNDYFEYFGPDFKLHISPSNMTNQNTNEYLE
KTKQRLFENLRMLPHAPGVQMQAIPEDATPEESGDEDEDDPDKRISICSSDKRI
ACEEEFSDSEEEGEGGRKNSSNFKKAKRVKTEDEKEKDPEEKKEVTEEEKTK
EEKPEAKGVKEEVKLA
Human HDAC1 coding sequence
(SEQ ID NO: 12)
atggcgcagacgcagggcacccggaggaaagtctgttactactacgacggggatgttggaaattactattattggac
aaggccacccaatgaagcctcaccgaatccgcatgactcataatttgctgctcaactatggtctctaccgaaaaatggaaat
ctatcgccctcacaaagccaatgctgaggagatgaccaagtaccacagcgatgactacattaaattcttgcgctccatccgt
ccagataacatgtcggagtacagcaagcagatgcagagattcaacgttggtgaggactgtccagtattcgatggcctgtttg
agttctgtcagttgtctactggtggttctgtggcaagtgctgtgaaacttaataagcagcagacggacatcgctgtgaattggg
ctgggggcctgcaccatgcaaagaagtccgaggcatctggcttctgttacgtcaatgatatcgtcttggccatcctggaact
gctaaagtatcaccagagggtgctgtacattgacattgatattcaccatggtgacggcgtggaagaggccttctacaccacg
gaccgggtcatgactgtgtcctttcataagtatggagagtacttcccaggaactggggacctacgggatatcggggctggc
aaaggcaagtattatgctgttaactacccgctccgagacgggattgatgacgagtcctatgaggccattttcaagccggtcat
gtccaaagtaatggagatgttccagcctagtgcggtggtcttacagtgtggctcagactccctatctggggatcggttaggtt
gcttcaatctaactatcaaaggacacgccaagtgtgtggaatttgtcaagagctttaacctgcctatgctgatgctgggaggc
ggtggttacaccattcgtaacgttgcccggtgctggacatatgagacagctgtggccctggatacggagatccctaatgag
cttccatacaatgactactttgaatactttggaccagatttcaagctccacatcagtccttccaatatgactaaccagaacacga
atgagtacctggagaagatcaaacagcgactgtttgagaaccttagaatgctgccgcacgcacctggggtccaaatgcag
gcgattcctgaggacgccatccctgaggagagtggcgatgaggacgaagacgaccctgacaagcgcatctcgatctgct
cctctgacaaacgaattgcctgtgaggaagagttctccgattctgaagaggagggagaggggggccgcaagaactcttcc
aacttcaaaaaagccaagagagtcaaaacagaggatgaaaaagagaaagacccagaggagaagaaagaagtcaccga
agaggagaaaaccaaggaggagaagccagaagccaaaggggtcaaggaggaggtcaagttggcctga
human HDAC2 protein sequence
(SEQ ID NO: 13)
MAYSQGGGKKKVCYYYDGDIGNYYYGQGHPMKPHRIRMTHNLLLNYG
LYRKMEIYRPHKATAEEMTKYHSDEYIKFLRSIRPDNMSEYSKQMQRFNVGE
DCPVFDGLFEFCQLSTGGSVAGAVKLNRQQTDMAVNWAGGLHHAKKSEASG
FCYVNDIVLAILELLKYHQRVLYIDIDIHHGDGVEEAFYTTDRVMTVSFHKYG
EYFPGTGDLRDIGAGKGKYYAVNFPMRDGIDDESYGQIFKPIISKVMEMYQPS
AVVLQCGADSLSGDRLGCFNLTVKGHAKCVEVVKTFNLPLLMLGGGGYTIR
NVARCWTYETAVALDCEIPNELPYNDYFEYFGPDFKLIDSPSNMTNQNTPEYM
EKIKQRLFENLRMLPHAPGVQMQAIPEDAVHEDSGDEDGEDPDKRISIRASDK
RIACDEEFSDSEDEGEGGRRNVADHKKGAKKARIEEDKKETEDKKTDVKEED
KSKDNSGEKTDTKGTKSEQLSNP
Human HDAC2 coding sequence
(SEQ ID NO: 14)
atggcgtacagtcaaggaggcggcaaaaaaaaagtctgctactactacgacggtgarattggaaattattattatgga
cagggtcatcccatgaagcctcatagaatccgcatgacccataacttgctgttaaattatggcttatacagaaaaatggaaat
atataggccccataaagccactgccgaagaaatgacaaaatatcacagtgatgagtatatcaaatttctacggtcaataaga
ccagataacatgtctgagtatagtaagcagatgcagagatttaatgttggagaagattgtccagtgtttgatggactctttgagt
tttgtcagctctcaactggcggttcagttgctggagctgtgaagttaaaccgacaacagactgatatggctgttaattgggctg
gaggattacatcatgctaagaaatcagaagcatcaggattctgttacgttaatgatattgtgcttgccatccttgaattactaaa
gtatcatcagagagtcttatatattgatatagatattcatcatggtgatggtgttgaagaagctttttatacaacagatcgtgtaat
gacggtatcattccataaatatggggaatactttcctggcacaggagacttgagggatattggtgctggaaaaggcaaatac
tatgctgtcaattttccaatgagagatggtatagatgatgagtcatatgggcagatatttaagcctattatctcaaaggtgatgg
agatgtatcaacctagtgctgtggtattacagtgtggtgcagactcattatctggtgatagactgggttgtttcaatctaacagtc
aaaggtcatgctaaatgtgtagaagttgtaaaaacttttaacttaccattactgatgcttggaggaggtggctacacaatccgt
aatgttgctcgatgttggacatatgagactgcagttgcccttgattgtgagattcccaatgagttgccatataatgattactttga
gtattttggaccagacttcaaactgcatattagtccttcaaacatgacaaaccagaacactccagaatatatggaaaagataa
aacagcgtttgtugaaaatttgcgcatgttacctcatgcacctggtgtccagatgcaagctattccagaagatgctgttcatga
agacagtggagatgaagatggagaagatccagacaagagaatttctattcgagcatcagacaagcggatagcttgtgatg
aagaattctcagattctgaggatgaaggagaaggaggtcgaagaaatgtggctgatcataagaaaggagcaaagaaagct
agaattgaagaagataagaaagaaacagaggacaaaaaaacagacgttaaggaagaagataaatccaaggacaacagt
ggtgaaaaaacagataccaaaggaaccaaatcagaacagctcagcaacccctga
human Kdm1a protein sequence
(SEQ ID NO: 15)
MLSGKKAAAAAAAAAAAATGTEAGPGTAGGSENGSEVAAQPAGLSGPA
EVGPGAVGERTPRKKEPPRASPPGGLAEPPGSAGPQAGPTVVPGSATPMETGI
AETPEGRRTSRRKRAKVEYREMDESLANLSEDEYYSEEERNAKAEKEKKLPP
PPPQAPPEEENESEPEEPSGQAGGLQDDSSGGYGDGQASGVEGAAFQSRLPH
DRMTSQEAACFPDIISGPQQTQKVFLFIRNRTLQLWLDNPKIQLTFEATLQQLE
APYNSDTVLVHRVHSYLERHGLINFGIYKRIKPLPTKKTGKVITIGSGVSGLAA
ARQLQSFGMDVTLLEARDRVGGRVATFRKGNYVADLGAMVVTGLGGNPMA
VVSKQVNMELAKIKQKCPLYEANGQADTVKVPKEKDEMVEQEFNRLLEATS
YLSHQLDFNVLNNKPVSLGQALEVVIQLQEKHVKDEQIEHWKKIVKTQEELK
ELLNKMVNLKEKIKELHQQYKEASEVKPPRDITAEFLVKSKHRDLTALCKEYD
ELAETQGKLEEKLQELEANPPSDVYLSSRDRQILDWHFANLEFANATPLSTLSL
KHWDQDDDFEFTGSHLTVRNGYSCVPVALAEGLDIKLNTAVRQVRYTASGCE
VIAVNTRSTSQTFIYKCDAVLCTLPLGVLKQQPPAVQFVPPLPEWKTSAVQRM
GFGNLNKVVLCFDRVFWDPSVNLFGHVGSTTASRGELFLFWNLYKAPILLAL
VAGEAAGIMENISDDVTVGRCLAILKGIFGSSAVPQPKETVVSRWRADPWARG
SYSYVAAGSSGNDYDLMAQPITPGPSIPGAPQPIPRLFFAGEHTTRNYPATVHG
ALLSGLREAGRIADQFLGAMYTLPRQATPGVPAQQSPSM
Human Kdm1a coding sequence
(SEQ ID NO: 16)
atgttatctgggaagaaggcggcagccgcggcggcggcggctgcagcggcagcaaccgggacggaggctgg
ccctgggacagcaggcggctccgagaacgggtctgaggtggccgcgcagcccgcgggcctgtcgggcccagccgag
gtcgggccgggggcggtgggggagcgcacaccccgcaagaaagagcctccgcgggcctcgccccccgggggcctg
gcggaaccgccggggtccgcagggcctcaggccggccctactgtcgtgcctgggtctgcgacccccatggaaactgga
atagcagagactccggaggggcgtcggaccagccggcgcaagcgggcgaaggtagagtacagagagatggatgaaa
gcttggccaacctctcagaagatgagtattattcagaagaagagagaaatgccaaagcagagaaggaaaagaagcttccc
ccaccaccccctcaagccccacctgaggaagaaaatgaaagtgagcctgaagaaccatcggggcaagcaggaggactt
caagacgacagttctggagggtatggagacggccaagcatcaggtgtggagggcgcagctttccagagccgacttcctc
atgaccggatgacttctcaagaagcagcctgttttccagatattatcagtggaccacaacagacccagaaggtttttcttttcat
tagaaaccgcacactgcagttgtggttggataatccaaagattcagctgacatttgaggctactctccaacaattagaagcac
cttataacagtgatactgtgcttgtccaccgagttcacagttatttagagcgtcatggtcttatcaacttcggcatctataagagg
ataaaacccctaccaactaaaaagacaggaaaggtaattattataggctctggggtctcaggcttggcagcagctcgacag
ttacaaagttttggaatggatgtcacacttttggaagccagggatcgtgtgggtggacgagttgccacatttcgcaaaggaaa
ctatgtagctgatcttggagccatggtggtaacaggtcttggagggaatcctatggctgtggtcagcaaacaagtaaatatg
gaactggccaagatcaagcaaaaatgcccactttatgaagccaacggacaagctgacactgtcaaggttcctaaagagaa
agatgaaatggtagagcaagagtttaaccggttgctagaagctacatcttaccttagtcatcaactagacttcaatgtcctcaa
taataagcctgtgtcccttggccaggcattggaagttgtcattcagttacaagagaagcatgtcaaagatgagcagattgaac
attggaagaagatagtgaaaactcaggaagaattgaaagaacttcttaataagatggtaaatttgaaagagaaaattaaaga
actccatcagcaatacaaagaagcatctgaagtaaagccacccagagatattactgccgagttcttagtgaaaagcaaaca
cagggatctgaccgccctatgcaaggaatatgatgaattagctgaaacacaaggaaagctagaagaaaaacttcaggagtt
ggaagcgaatcccccaagtgatgtatatctctcatcaagagacagacaaatacttgattggcattttgcaaatcttgaatttgct
aatgccacacctctctcaactctctcccttaagcactgggatcaggatgatgactttgagttcactggcagccacctgacagt
aaggaatggctactcgtgtgtgcctgtggctttagcagaaggcctagacattaaactgaatacagcagtgcgacaggttcgc
tacacggcttcaggatgtgaagtgatagctgtgaatacccgctccacgagtcaaacctttatttataaatgcgacgcagttctc
tgtacccttcccctgggtgtgctgaagcagcagccaccagccgttcagtttgtgccacctctccctgagtggaaaacatctg
cagtccaaaggatgggatttggcaaccttaacaaggtggtgttgtgttttgatcgggtgttctgggatccaagtgtcaatttgtt
cgggcatgttggcagtacgactgccagcaggggtgagctcttcctcttctggaacctctataaagctccaatactgttggcac
tagtggcaggagaagctgctggtatcatggaaaacataagtgacgatgtgattgttggccgatgcctggccattctcaaagg
gatttttggtagcagtgcagtacctcagcccaaagaaactgtggtgtctcgttggcgtgctgatccctgggctcggggctctt
attcctatgttgctgcaggatcatctggaaatgactatgatttaatggctcagccaatcactcctggcccctcgattccaggtgc
cccacagccgattccacgactcttctttgcgggagaacatacgatccgtaactacccagccacagtgcatggtgctctgctg
agtgggctgcgagaagcgggaagaattgcagaccagtttttgggggccatgtatacgctgcctcgccaggccacaccag
gtgttcctgcacagcagtccccaagcatgtga
human PHF21A protein sequence
(SEQ ID NO: 17)
MELQTLQEALKVEIQVHQKLYAQMKQDPQNADLKKQLHELQAKITALS
EKQKRVVEQLRKNLTVKQEQPDKFQIQPLPQSENKLQTAQQQPLQQLQQQQQ
YHHHHAQQSAAASPNLTASQKTVTTASMITTKTLPLVLKAATATMPASVYGQ
RPTIAMVTAINSQKAVLSTDVQNTPVNLQTSSKVTGPGAEAVQIVAKNTVTLV
QATPPQPIKVPQFIPPPRLTPRPNFLPQVRPKPVAQNNIPIAPAPPPMLAAPQLIQ
RPVMLTKFTPTTLPTSQNSIHPVRVVNGQTATIAKTFPMAQLTSIVIATPGTRLA
GPQTVQLSKPSLEKQTVKSHTETDEKQTESRTITPPAAPKPKREENPQKLAFM
VSLGLVTHDHLEEIQSKRQERKRRTTANPVYSGAVFEPERKKSAVTYLNSTMH
PGTRKRGRPPKYNAVLGFGALTPTSPQSSHPDSPENEKTETTFTFPAPVQPVSL
PSPTSTDGDIHEDFCSVCRKSGQLLMCDTCSRVYHLDCLDPPLKTIPKGMWIC
PRCQDQMLKKEEAIPWPGTLAIVHSYIAYKAAKIEEEKQKLLKWSSDLKQERE
QLEQKVKQLSNSISKCMEMKNTILARQKEMHSSLEKVKQLIRLIHGIDLSKPV
DSEATYGAISNGPDCTPPANAATSTPAPSPSSQSCTANCNQGEETK
Human PHF21A coding sequence
(SEQ ID NO: 18)
atggagttgcagactctacaggaggctcttaaagtggaaattcaggttcaccagaaactggttgctcaaatgaagca
ggatccacagaatgctgacttaaagaaacagcttcatgaactccaagccaaaatcacagctttgagtgagaaacagaaaag
agtagttgaacagctacggaagaacctgatagtaaagcaagaacaaccggacaagttccaaatacagccattgccacaat
ctgaaaacaaactacaaacagcacagcagcaaccactacagcaactacaacaacagcagcagtaccaccaccaccacg
cccagcagtcagctgcagcctctcccaacctgactgcttcacagaagactgtaactacagcttctatgattaccacaaagac
actacctctcgtcttgaaagcagcaactgcgaccatgcctgcctctgtggtgggccagagacctaccattgctatggtgacc
gccatcaacagtcagaaggctgtgctcagcactgatgtgcagaacacaccagtcaacctccagacgtctagtaaggtcact
gggcctggggcagaggctgtccaaattgtggcaaaaaacacagtcactctggttcaggcaacacctcctcagcccatcaa
agtaccacagtttatcccccctcctagactcactccacgtccaaactttcttccacaggttcgacccaagcctgtggcccaga
ataacattcctattgccccagcaccacctcccatgctcgcagctcctcagcttatccagaggcccgtcatgctgaccaagttc
acccccacaacccttcccacatcccagaattccatccaccccgtccgtgtcgtcaatgggcagactgcaaccatagccaaa
acgttccccatggcccagcrcaccagcattgtgatagctactccagggaccagactcgctggacctcaaactgtacagctta
gcaagccaagtcttgaaaaacagacagttaaatctcacacagaaacagatgagaaacaaacagagagccgcaccatcac
cccacctgctgcacccaaaccaaaacgggaggagaaccctcagaaacttgccttcatggtgtctctagggttggtaacaca
tgaccatctagaagaaatccaaagcaagaggcaagagcgaaaaagaagaacaacagcaaatccggtctacagtggagc
agtctttgagccagagcgtaagaagagtgcagtgacatacctaaacagcacaatgcaccctgggacccggaagagaggt
cgtcctccaaaatacaatgcagtgctggggtttggagcccttaccccaacatccccccaatccagtcatcctgactcccctg
aaaatgaaaagacagagaccacattcactttccctgcacctgttcagcctgtgtccctgcccagccccacctccacagacg
gtgatattcatgaggatttttgcagcgtttgcagaaaaagtggccagttactgatgtgcgacacatgttcccgtgtatatcatttg
gactgcttagacccccctctgaaaacaattcccaagggcatgtggatctgtcccagatgtcaggaccagatgctgaagaag
gaagaagcaattccatggcctggaactttagcaattgttcattcctatattgcctacaaagcagcaaaagaagaagagaaac
agaagttacttaaatggagttcagatttaaaacaagaacgagaacaactagagcaaaaggtgaaacagctcagcaattcca
taagtaaatgcatggaaatgaagaacaccatcctggcccggcagaaggagatgcacagctccctggagaaggtaaaaca
gctgattcgcctcatccacggcatcgacctctccaaacctgtagactctgaggccactgtgggggccatctccaatggccc
ggactgcaccccccctgccaatgccgccacctccacgccggccccttccccctcctcccagagctgcacagcgaactgta
accagggggaagagactaaataa
human BAF53a protein sequence
(SEQ ID NO: 19)
MSGGVYGGDEVGALVFDIGSYTVRAGYAGEDCPKVDFPTAIGMVVERD
DGSTLMEIDGDKGKQGGPTYYIDTNALRVPRENMEAISPLKNGMVEDWDSF
QAILDHTYKMHYKSEASLHPVLMSEAPWNTRAKREKLTELMFEHYNIPAFFL
CKTAVLTAFANGRSTGLILDSGATHTTAIPVHDGYVLQQGIVKSPLAGDFITMQ
CRELFQEMNIELVPPYMIASKEAVREGSPANWKRKEKLPQVTRSWHNYMCNC
VIQDFQASVLQVSDSTYDEQVAAQMPTVHYEFPNGYNCDFGAERLKIPEGLF
DPSNVKGLSGNTMLGVSHVVTTSVGMCDIDIRPGLYGSVIYAGGNTLIQSFTD
RLNRELSQKTPPSMRLKLIANNTTVERRFSSWIGGSILASLGTFQQMWISKQE
YEEGGKQCVERKCP
Human BAF53a coding sequence
(SEQ ID NO: 20)
atgagcggcggcgtgtacgggggagatgaagttggagcccttgtttttgacattggatcctatactgtgagagctggt
tatgctggtgaggactgccccaaggtggattttcctacagctattggtatggtggtagaaagagatgacggaagcacattaat
ggaaatagatggcgataaaggcaaacaaggcggtcccacctactacatagatactaatgctctgcgtgttccgagggaga
atatggaggccatttcacctctaaaaaatgggatggttgaagactgggatagtttccaagctattttggatcatacctacaaaat
gcatgtcaaatcagaagccagtctccatcctgttctcatgtcagaggcaccgtggaatactagagcaaagagagagaaact
gacagagttaatgtttgaacactacaacatccctgccttcttcctttgcaaaactgcagttttgacagcatttgctaatggtcgtt
ctactgggctgattttggacagtggagccactcataccactgcaattccagtccacgatggctatgtccttcaacaaggcatt
gtgaaatcccctcttgctggagactttattactatgcagtgcagagaactcttccaagaaatgaatattgaattggttcctccata
tatgattgcatcaaaagaagctgttcgtgaaggatctccagcaaactggaaaagaaaagagaagttgcctcaggttacgag
gtcttggcacaattatatgtgtaattgtgttatccaggattttcaagcttcggtacttcaagtgtcagattcaacttatgatgaaca
agtggctgcacagatgccaactgttcattatgaattccccaatggctacaattgtgattttggtgcagagcggctaaagattcc
agaaggattatttgacccttccaatgtaaaggggttatcaggaaacacaatgttaggagtcagtcatgttgtcaccacaagtgt
tgggatgtgtgatattgacatcagaccaggtctctatggcagtgtaatagtggcaggaggaaacacactaatacagagtttta
ctgacaggttgaatagagagctgtctcagaaaactcctccaagtatgcggttgaaattgattgcaaataatacaacagtggaa
cggaggtttagctcatggattggcggctccattctagcctctttgggtacctttcaacagatgtggatttccaagcaagaatat
gaagaaggagggaagcagtgtgtagaaagaaaatgcccttga
human G9a protein sequence
(SEQ ID NO: 21)
MAAAAGAAAAAAAEGEAPAEMGALLLEKETRGATERVHGSLGDTPRSE
ETLPKATPDSLEPAGPSSPASVTVTVGDEGADTPVGATPLIGDESENLEGDGDL
RGGRILLGHATKSFPSSPSKGGSCPSRAKMSMTGAGKSPPSVQSLAMRLLSMP
GAQGAAAAGSEPPPAITSPEGQPKVHRARKTMSKPGNGQPPVPEKRPPEIQHF
RMSDDVHSLGKVTSDLAKRRKLNSGGGLSEELGSARRSGEVTLTKGDPGSLE
EWETYYGDDFSLYYDSYSYDERYDSDSKSEYEALTEQLSEEEEEEEEEEEEEE
EEEEEEEEEEDEESGNQSDRSGSSGRRKAKKKWRKDSPWVKPSRKRRKREPP
RAKEPRGVNGVGSSGPSEYMEVPLGSLELPSEGTLSPNHAGVSNDTSSLETER
GFEELPLCSCRMEAPKIDRISERAGHKCMATESVDGELSGCNAAILKRETMRP
SSRVALMVLCETHRARMVKHHCCPGCGYFCTAGTFLECHPDFRVAHRFHKAC
VSQLNGMVFCPHCGEDASEAQEVTIPRGDGVTPPAGTAAPAPPPLSQDVPGRA
DTSQPSARMRGHGEPRRPPCDPLADTIDSSGPSLTLPNGGCLSAVGLPLGPGRE
ALEKALVIQESERRKKLRFHPRQLYLSVKQGELQKVILMLLDNLDPNFQSDQQ
SKRTPLHAAAQKGSVEICHVLLQAGANINAVDKQQRTPLMEAVVNNHLEVAR
YMVQRGGCVYSKEEDGSTCLHHAAKIGNLEMVSLLLSTGQVDVNAQDSGG
WTPIIWAAEHKHIEVIRMLLTRGADYTLTDNEENICLHWASFTGSAAIAEVLLN
ARCDLHAVNYHGDTPLHIAARESYHDCVLLFLSRGANPELRNKEGDTAWDLT
PERSDVWFALQLNRKLRLGVGNRAIRTEKIICRDVARGYENVPIPCVNGVDGE
PCPEDYKYISENCETSTMNIDRNITHLQHCTCVDDCSSSNCLCGQLSIRCWYD
KDGRLLQEFNKIEPPLIFECNQACSCWRNCKNRVVQSGIKVRLQLYRTAKMG
WGVRALQTIPQGTFICEYVGELISDAEADVREDDSYLFDLDNKDGEVYCIDA
RYYGNISRFINHLCDPNIIPVRVFMLHQDLRFPRIAFFSSRDIRTGEELGFDYGD
RFWDIKSKYFTCQCGSEKCKHSAEAIALEQSRLARLDPHPELLPELGSLPPVNT
Human G9a coding sequence
(SEQ ID NO: 22)
atggcggcggcggcgggagctgcagcggcggcggccgccgagggggaggcccccgctgagatgggggcgc
tgctgctggagaaggaaaccagaggagccaccgagagagttcatggctctttgggggacacccctcgtagtgaagaaac
cctgcccaaggccacccccgactccctggagcctgctggcccctcatctccagcctctgtcactgtcactgttggtgatgag
ggggctgacacccctgtaggggctacaccactcattggggatgaatctgagaatcttgagggagatggggacctccgtgg
gggccggatcctgctgggccatgccacaaagtcattcccctcttcccccagcaaggggggttcctgtcctagccgggcca
agatgtcaatgacaggggcgggaaaatcacctccatctgtccagagtttggctatgaggctactgagtatgccaggagccc
agggagctgcagcagcagggtctgaaccccctccagccaccacgagcccagagggacagcccaaggtccaccgagc
ccgcaaaaccatgtccaaaccaggaaatggacagcccccggtccctgagaagcggccccctgaaatacagcatttccgc
atgagtgatgatgtccactcactgggaaaggtgacctcagatctggccaaaaggaggaagctgaactcaggaggtggcct
gtcagaggagttaggttctgcccggcgttcaggagaagtgaccctgacgaaaggggaccccgggtccctggaggagtg
ggagacggtggtgggtgatgacttcagtctctactatgattcctactctgtggatgagcgcgtggactccgacagcaagtct
gaagttgaagctctaactgaacaactaagtgaagaggaggaggaggaagaggaggaagaagaagaagaggaagagg
aggaggaagaggaagaagaagaggaagatgaggagtcagggaatcagtcagataggagtggttccagtggccggcgc
aaggccaagaagaaatggcgaaaagacagcccatgggtgaagccgtctcggaaacggcgcaagcgggagcctccgc
gggccaaggagccacgaggagtgaatggtgtgggctcctcaggccccagtgagtacatggaggtccctctggggtccct
ggagctgcccagcgaggggaccctctcccccaaccacgctggggtgtccaatgacacatcttcgctggagacagagcga
gggtttgaggagttgcccctgtgcagctgccgcatggaggcacccaagattgaccgcatcagcgagagggcggggcac
aagtgcatggccactgagagtgtggacggagagctgtcaggctgcaatgccgccatcctcaagcgggagaccatgagg
ccatccagccgtgtggccctgatggtgctctgtgagacccaccgcgcccgcatggtcaaacaccactgctgcccgggctg
cggctacttctgcacggcgggcaccttcctggagtgccaccctgacttccgtgtggcccaccgcttccacaaggcctgtgt
gtctcagctgaatgggatggtcttctgtccccactgtggggaggatgcttctgaagctcaagaggtgaccatcccccggggt
gacggggtgaccccaccggccggcactgcagctcctgcacccccacccctgtcccaggatgtccccgggagagcagac
acttctcagcccagtgcccggatgcgagggcatggggaaccccggcgcccgccctgcgatcccctggctgacaccattg
acagctcagggccctccctgaccctgcccaatgggggctgcctttcagccgtggggctgccactggggccaggccggg
aggccctggaaaaggccctggtcatccaggagtcagagaggcggaagaagctccgtttccaccctcggcagttgtacctg
tccgtgaagcagggcgagctgcagaaggtgatcctgatgctgttggacaacctggaccccaacttccagagcgaccagc
agagcaagcgcacgcccctgcatgcagccgcccagaagggctccgtggagatctgccatgtgctgctgcaggctggag
ccaacataaatgcagtggacaaacagcagcggacgccactgatggaggccgtggtgaacaaccacctggaggtagccc
gttacatggtgcagcgtggtggctgtgtctatagcaaggaggaggacggttccacctgcctccaccacgcagccaaaatc
gggaacttggagatggtcagcctgctgctgagcacaggacaggtggacgtcaacgcccaggacagtggggggtggac
gcccatcatctgggctgcagagcacaagcacatcgaggtgatccgcatgctactgacgcggggcgccgacgtcaccctc
actgacaacgaggagaacatctgcctgcactgggcctccttcacgggcagcgccgccatcgccgaagtccttctgaatgc
gcgctgtgacctccatgctgtcaactaccatggggacacccccctgcacatcgcagctcgggagagctaccatgactgcg
tgctgttattcctgtcacgtggggccaaccctgagctgcggaacaaagagggggacacagcatgggacctgactcccga
gcgctccgacgtgtggtttgcgcttcaactcaaccgcaagctccgacttggggtgggaaatcgggccatccgcacagaga
agatcatctgccgggacgtggctcggggctatgagaacgtgcccattccctgtgtcaacggtgtggatggggagccctgc
cctgaggattacaagtacatctcagagaactgcgagacgtccaccatgaacatcgatcgcaacatcacccacctgcagca
ctgcacgtgtgtggacgactgctctagctccaactgcctgtgcggccagctcagcatccggtgctggtatgacaaggatgg
gcgattgctccaggaatttaacaagattgagcctccgctgattttcgagtgtaaccaggcgtgctcatgctggagaaactgca
agaaccgggtcgtacagagtggcatcaaggtgcggctacagctctaccgaacagccaagatgggctggggggtccgcg
ccctgcagaccatcccacaggggaccttcatctgcgagtatgtcggggagctgatctctgatgctgaggctgatgtgagag
aggatgattcttacctcttcgacttagacaacaaggatggagaggtgtactgcatagatgcccgttactatggcaacatcagc
cgcttcatcaaccacctgtgtgaccccaacatcattcccgtccgggtcttcatgctgcaccaagacctgcgatttccacgcat
cgccttcttcagttcccgagacatccggactggggaggagctagggtttgactatggcgaccgcttctgggacatcaaaag
caaatatttcacctgccaatgtggctctgagaagtgcaagcactcagccgaagccattgccctggagcagagccgtctggc
ccgcctggacccacaccctgagctgctgcccgagctcggctccctgccccctgtcaacacatga
human USP14 protein sequence
(SEQ ID NO: 23)
MPLYSVTVKWGKEKFEGYELNTDEPPMVFKAQLFALTGVQPARQKVMV
KGGTLKDDDWGNIKIKNGMTLLMMGSADALPEEPSAKTVFVEDMTEEQLAS
AMELPCGLTNLGNTCYMNATYQCIRSYPELKDALKRYAGALRASGEMASAQ
YITAALRDLFDSMDKTSSSIPPIILLQFLHMAFPQFAEKGEQGQYLQQDANEC
WIQMMRVLQQKLEAIEDDSVKETDSSSASAATPSKKKSLIDQFFGVEFETTMK
CTESEEEEVTKGKENQLQLSCFINQEVKYLFTGLKLRLQEEITKQSPTLQRNAL
YIKSSKISRLPAYLTIQMVRFFYKEKESVNAKYLKDVKFPLMLDMYELCTPEL
QEKMVSFRSKFKDLEDKKVNQQPNTSDKKSSPQKEVKYEPFSFADDIGSNNC
GYYDLQAVLTHQGRSSSSGHYVSWVKRKQDEWTKFDDDKVSIVTPEDILRLS
GGGDWHIAYVLLYGPRRVEIMEEESEQ
Human USP14 coding sequence
(SEQ ID NO: 24)
atgccgctctactccgttactgtaaaatggggaaaggagaaatttgaaggtgtagaattgaatacagatgaacctcca
atggtattcaaggctcagctgtttgcgttgactggagtccagcctgccagacagaaagttatggtgaaaggaggaacgcta
aaggatgatgattggggaaacatcaaaataaaaaatggaatgactctactaatgatggggtcagcagatgctcttccagaag
aaccctcagccaaaactgtcttcgtagaagacatgacagaagaacagttagcatctgctatggagttaccatgtggattgac
aaaccttggtaacacttgttacatgaatgccacagttcagtgtattcgttctgtgcctgaactcaaagatgcccttaaaaggtat
gcaggtgccttgagagcttcaggggaaatggcttcagcgcagtatattactgcagcccttagagatttgtttgattccatggat
aaaacttcttccagtattccacctattattctactgcagtttttgcacatggctttcccacagtttgccgagaaaggtgaacaagg
acagtatcttcaacaggatgctaatgaatgttggatacaaatgatgcgagtattgcaacagaaattggaagcaatagaggat
gattctgttaaagagacagactcctcatctgcatcggcagcgacaccttctaaaaagaaaagtttaatcgatcagttcttcggt
gttgagtttgaaactaccatgaaatgtacagaatctgaagaagaagaagtcaccaaaggaaaggaaaatcaacttcagctta
gctgttttatcaatcaggaagtcaagtatctttttacaggacttaaattgcgacttcaggaagaaatcaccaaacagtctccaac
gttgcaaagaaatgccttgtatatcaaatcttccaagatcagccggctgcctgcttacttgaccattcagatggttcgattttttta
taaagagaaggaatctgtgaatgccaaagttcttaaggatgttaaatttcctcttatgttggatatgtatgaactgtgtacaccag
aacttcaagagaaaatggtgtcttttcgatccaaattcaaggatctagaagataaaaaagtgaatcagcagccaaatacaagt
gacaaaaagagtagtccccagaaagaagttaagtatgaacccttttcttttgctgatgatattggctccaataattgtggatact
atgacttacaagcagtactaacacaccagggaaggtctagttcttcaggtcattatgtatcatgggtgaaaaggaaacaagat
gaatggattaagtttgatgatgacaaagtcagcatcgtaacaccagaagatatcttacggctttctggtggtggagactggca
tatcgcttacgttctactctatgggcctcgcagagttgaaataatggaagaggaaagtgaacagtaa
human HuR protein sequence
(SEQ ID NO: 25)
MSNGYEDHMAEDCRGDIGRTNLIVNYLPQNMTQDELRSLFSSIGEVESA
KLIRDKVAGHSLGYGFVNYVTAKDAERAINTLNGLRLQSKTIKVSYARPSSEV
IKDANLYISGLPRTMTQKDVEDMFSRFGRHNSRVLVDQTTGLSRGVAFIRFDK
RSEAEEAITSFNGHKPPGSSEPITVKFAANPNQNKNVALLSQLYHSPARRFGGP
VHHQAQRFRFSPMGVDHMSGLSGVNVPGNASSGWCIFIYNLGQDADEGILW
QMFGPFGAVTNVKVIRDFNTNKCKGFGFVTMTNYEEAAMAIASLNGYRLGD
KILQVSFKTNKSHK
Human HuR coding sequence
(SEQ ID NO: 26)
atgtctaatggttatgaagaccacatggccgaagactgcaggggtgacatcgggagaacgaatttgatcgtcaacta
cctccctcagaacatgacccaggatgagttacgaagcctgttcagcagcattggtgaagttgaatctgcaaaacttattcgg
gataaagtagcaggacacagcttgggctatggctttgtgaactacgtgaccgcgaaggatgcagagagagcgatcaacac
gctgaacggcttgaggctccagtcaaaaaccattaaggtgtcgtatgctcgcccgagctcagaggtgatcaaagacgcca
acttgtacatcagcgggctcccgcggaccatgacccagaaggacgtagaagacatgttctctcggtttgggcggatcatca
actcgcgggtcctcgtggatcagactacaggtttgtccagaggggttgcgtttatccggtttgacaaacggtcggaggcag
aagaggcaattaccagtttcaatggtcataaacccccaggttcctctgagcccatcacagtgaagtttgcagccaaccccaa
ccagaacaaaaacgtggcactcctctcgcagctgtaccactcgccagcgcgacggttcggaggccccgttcaccaccag
gcgcagagattcaggttctcccccatgggcgtcgatcacatgagcgggctctctggcgtcaacgtgccaggaaacgcctc
ctccggctggtgcattttcatctacaacctggggcaggatgccgacgaggggatcctctggcagatgtttgggccgtttggt
gccgtcaccaatgtgaaagtgatccgcgacttcaacaccaacaagtgcaaagggtttggctttgtgaccatgacaaactatg
aagaagccgcgatggccatagccagcctgaacggctaccgcctgggggacaaaatcttacaggtttccttcaaaaccaac
aagtcccacaaataa
human BrG1 protein sequence
(SEQ ID NO: 27)
MSTPDPPLGGTPRPGPSPGPGPSPGAMLGPSPGPSPGSAHSMMGPSPGPPS
AGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPGGA
PLDGADPQALGQQNRGPTPFNQNQLHQLRAQIMAYKMLARGQPLPDHLQM
AVQGKRPMPGMQQQMPTLPPPSVSATGPGPGPGPGPGPGPGPAPPNYSRPHG
MGGPNMPPPGPSGVPPGMPGQPPGGPPKPWPEGPMANAAAPTSTPQKLIPPQ
PTGRPSPAPPAVPPAASPVMPPQTQSPGQPAQPAPMVPLHQKQSRITPIQKPRGL
DPVEILQEREYRLQARIAHRIQELENLPGSLAGDLRTKATIELKALRLLNFQRQ
LRQEVVVCMRRDTALETALNAKAYKRSKRQSLREARITEKLEKQQKIEQERK
RRQKHQEYLNSILQHAKDFKEYHRSVTGKIQKLTKAVATYHANTEREQKKEN
ERIEKERMRRLMAEDEEGYRKLIDQKKDKRLAYLLQQTDEYVANLTELVRQH
KAAQYAKEKKKKKKKKKAENAEGQTPAIGPDGEPLDETSQMSDLPYKVIHV
ESGKILTGTDAPKAGQLEAWLEMNPGYEVAPRSDSEESGSEEEEEEEEEEQPQ
AAQPPTLPVEEKKKIPDPDSDDYSEYDARHIIENAKQDYDDEYGYSQALARG
LQSYYAVAHAVTERVDKQSALMVNGVLKQYQIKGLEWLVSLYNNNLNGILA
DEMGLGKTIQTLALITYLMEHKRINGPFLIIVPLSTLSNWAYEFDKWAPSVVKV
SYKGSPAARRAFVPQLRSGKFNVLLTTYEYIIKDKHILAKIRWKYMIVDEGHR
MKNHHCKLTQVLNTHYVAPRRLLLTGTPLQNKLPELWALLNFLLPTIFKSCST
FEQWFNAPFAMTGEKVDLNEEETILIIRRLHKVLRPFLLRRLKKEVEAQLPEK
VEYVIKCDMSALQRYLYRHMQAKGVLLTDGSEKDKKGKGGTKTLMNTIMQ
LRKICNHPYMFQHIEESFSEHLGFTGGIVQGLDLYRASGKFELLDRILPKLRAT
NHKYLLFCQMTSLMTIMEDYFAYRGFKYLRLDGTTKAEDRGMLLKTFNEPG
SEYFIFLLSTRAGGLGLNLQSADTYIIFDSDWNPHQDLQAQDRAHRIGQQNEV
RYLRLCTYNSVEKTLAAAKYKLNYDQKVIQAGMFDQKSSSHERRAFLQAIL
EHEEQDESRHCSTGSGSASFAHTAPPPAGVNPDLEEPPLKEEDEVPDDETYNQ
MIARHEEEFDLFMRMDLDRRREEARNPKRKPRLMEEDELPSWIIKDDAEVER
LTCEEEEEKMFGRGSRHRKEVDYSDSLTEKQWLKKITGKDIHDTASSYARGLQ
FQRGLQFCTRASKAIEEGTLEEIEEEVRQKKSSRKRKRDSDAGSSTPTTSTRSR
DKDDESKKQKKRGRPPAEKLSPNPPNLTKKMKKIVDAYIKYKDSSSGRQLSEY
FIQLPSRKELPEYYELIRKPVDFKKIKERIRNHKYRSLNDLEKDVMLLCQNAQ
TFNLEGSLIYEDSIVLQSVFTSVRQKIEKEDDSEGEESEEEEEGEEEGSESESRS
VKVKIKLGRKEKAQDRLKGGRRRPSRGSRAKPVVSDDDSEEEQEEDRSGSGS
EED
Human BrG1 coding sequence
(SEQ ID NO: 28)
atgtccactccagacccacccctgggcggaactcctcggccaggtccttccccgggccctggcccttcccctgga
gccatgctgggccctagcccgggtccctcgccgggctccgcccacagcatgatggggcccagcccagggccgccctca
gcaggacaccccatccccacccaggggcctggagggtaccctcaggacaacatgcaccagatgcacaagcccatgga
gtccatgcatgagaagggcatgtcggacgacccgcgctacaaccagatgaaaggaatggggatgcggtcagggggcca
tgctgggatggggcccccgcccagccccatggaccagcactcccaaggttacccctcgcccctgggtggctctgagcat
gcctctagtccagttccagccagtggcccgtcttcggggccccagatgtcttccgggccaggaggtgccccgctggatgg
tgctgacccccaggccttggggcagcagaaccggggcccaaccccatttaaccagaaccagctgcaccagctcagagct
cagatcatggcctacaagatgctggccagggggcagcccctccccgaccacctgcagatggcggtgcagggcaagcg
gccgatgcccgggatgcagcagcagatgccaacgctacctccaccctcggtgtccgcaacaggacccggccctggccc
tggccctggccccggcccgggtcccggcccggcacctccaaattacagcaggcctcatggtatgggagggcccaacat
gcctcccccaggaccctcgggcgtgccccccgggatgccaggccagcctcctggagggcctcccaagccctggcctga
aggacccatggcgaatgctgctgcccccacgagcacccctcagaagctgattcccccgcagccaacgggccgcccttcc
cccgcgccccctgccgtcccacccgccgcctcgcccgtgatgccaccgcagacccagtcccccgggcagccggccca
gcccgcgcccatggtgccactgcaccagaagcagagccgcatcacccccatccagaagccgcggggcctcgaccctgt
ggagatcctgcaggagcgcgagtacaggctgcaggctcgcatcgcacaccgaattcaggaacttgaaaaccttcccggg
tccctggccggggatttgcgaaccaaagcgaccattgagctcaaggccctcaggctgctgaacttccagaggcagctgcg
ccaggaggtggtggtgtgcatgcggagggacacagcgctggagacagccctcaatgctaaggcctacaagcgcagcaa
gcgccagtccctgcgcgaggcccgcatcactgagaagctggagaagcagcagaagatcgagcaggagcgcaagcgc
cggcagaagcaccaggaatacctcaatagcattctccagcatgccaaggatttcaaggaatatcacagatccgtcacaggc
aaaatccagaagctgaccaaggcagtggccacgtaccatgccaacacggagcgggagcagaagaaagagaacgagc
ggatcgagaaggagcgcatgcggaggctcatggctgaagatgaggaggggtaccgcaagctcatcgaccagaagaag
gacaagcgcctggcctacctcttgcagcagacagacgagtacgtggctaacctcacggagctggtgcggcagcacaag
gctgcccaggtcgccaaggagaaaaagaagaaaaagaaaaagaagaaggcagaaaatgcagaaggacagacgcctg
ccattgggccggatggcgagcctctggacgagaccagccagatgagcgacctcccggtgaaggtgatccacgtggaga
gtgggaagatcctcacaggcacagatgcccccaaagccgggcagctggaggcctggctcgagatgaacccggggtatg
aagtagctccgaggtctgatagtgaagaaagtggctcagaagaagaggaagaggaggaggaggaagagcagccgcag
gcagcacagcctcccaccctgcccgtggaggagaagaagaagattccagatccagacagcgatgacgtctctgaggtgg
acgcgcggcacatcattgagaatgccaagcaagatgtcgatgatgaatatggcgtgtcccaggcccttgcacgtggcctg
cagtcctactatgccgtggcccatgctgtcactgagagagtggacaagcagtcagcgcttatggtcaatggtgtcctcaaac
agtaccagatcaaaggtttggagtggctggtgtccctgtacaacaacaacctgaacggcatcctggccgacgagatgggc
ctggggaagaccatccagaccatcgcgctcatcacgtacctcatggagcacaaacgcatcaatgggcccttcctcatcatc
gtgcctctctcaacgctgtccaactgggcgtacgagtttgacaagtgggccccctccgtggtgaaggtgtcttacaagggat
ccccagcagcaagacgggcctttgtcccccagctccggagtgggaagttcaacgtcttgctgacgacgtacgagtacatc
atcaaagacaagcacatcctcgccaagatccgttggaagtacatgattgtggacgaaggtcaccgcatgaagaaccacca
ctgcaagctgacgcaggtgctcaacacgcactatgtggcaccccgccgcctgctgctgacgggcacaccgctgcagaac
aagcttcccgagctctgggcgctgctcaacttcctgctgcccaccatcttcaagagctgcagcaccttcgagcagtggtttaa
cgcaccctttgccatgaccggggaaaaggtggacctgaatgaggaggaaaccattctcatcatccggcgtctccacaaag
tgctgcggcccttcttgctccgacgactcaagaaggaagtcgaggcccagttgcccgaaaaggtggagtacgtcatcaag
tgcgacatgtctgcgctgcagcgagtgctctaccgccacatgcaggccaagggcgtgctgctgactgatggctccgagaa
ggacaagaagggcaaaggcggcaccaagaccctgatgaacaccatcatgcagctgcggaagatctgcaaccaccccta
catgttccagcacatcgaggagtccttttccgagcacttggggttcactggcggcattgtccaagggctggacctgtaccga
gcctcgggtaaatttgagcttcttgatagaattcttcccaaactccgagcaaccaaccacaaagtgctgctgttctgccaaatg
acctccctcatgaccatcatggaagattactttgcgtatcgcggctttaaatacctcaggcttgatggaaccacgaaggcgga
ggaccggggcatgctgctgaaaaccttcaacgagcccggctctgagtacttcatcttcctgctcagcacccgggctgggg
ggctcggcctgaacctccagtcggcagacactgtgatcatttttgacagcgactggaatcctcaccaggacctgcaagcgc
aggaccgagcccaccgcatcgggcagcagaacgaggtgcgtgtgctccgcctctgcaccgtcaacagcgtggaggag
aagatcctagctgcagccaagtacaagctcaacgtggaccagaaggtgatccaggccggcatgttcgaccagaagtcctc
cagccatgagcggcgcgccttcctgcaggccatcctggagcacgaggagcaggatgagagcagacactgcagcacgg
gcagcggcagtgccagcttcgcccacactgcccctccgccagcgggcgtcaaccccgacttggaggagccacctctaaa
ggaggaagacgaggtgcccgacgacgagaccgtcaaccagatgatcgcccggcacgaggaggagtttgatctgttcat
gcgcatggacctggaccgcaggcgcgaggaggcccgcaaccccaagcggaagccgcgcctcatggaggaggacga
gctcccctcgtggatcatcaaggacgacgcggaggtggagcggctgacctgtgaggaggaggaggagaagatgttcgg
ccgtggctcccgccaccgcaaggaggtggactacagcgactcactgacggagaagcagtggctcaagaaaattacagg
aaaagatatccatgacacagccagcagtgtggcacgtgggctacaattccagcgtggccttcagttctgcacacgtgcgtc
aaaggccatcgaggagggcacgctggaggagatcgaagaggaggtccggcagaagaaatcatcacggaagcgcaag
cgagacagcgacgccggctcctccaccccgaccaccagcacccgcagccgcgacaaggacgacgagagcaagaagc
agaagaagcgcgggcggccgcctgccgagaaactctcccctaacccacccaacctcaccaagaagatgaagaagattg
tggatgccgtgatcaagtacaaggacagcagcagtggacgtcagctcagcgaggtcttcatccagctgccctcgcgaaag
gagctgcccgagtactacgagctcatccgcaagcccgtggacttcaagaagataaaggagcgcattcgcaaccacaagt
accgcagcctcaacgacctagagaaggacgtcatgctcctgtgccagaacgcacagaccttcaacctggagggctccct
gatctatgaagactccatcgtcttgcagtcggtcttcaccagcgtgcggcagaaaatcgagaaggaggatgacagtgaag
gcgaggagagtgaggaggaggaagagggcgaggaggaaggctccgaatccgaatctcggtccgtcaaagtgaagatc
aagcttggccggaaggagaaggcacaggaccggctgaagggcggccggcggcggccgagccgagggtcccgagcc
aagccggtcgtgagtgacgatgacagtgaggaggaacaagaggaggaccgctcaggaagtggcagcgaagaagactg
a
human EZH2 protein sequence
(SEQ ID NO: 29)
MGQTGKKSEKGPVCWRKRVKSEYMRLRQLKRFRRADEVKSMFSSNRQ
KILERTEILNQEWKQRRIQPVHILTSVSSLRGTRECSVTSDLDFPTQVIPLKTLN
AVASVPIMYSWSPLQQNFMVEDETVLHNIPYMGDEVLDQDGTFIEELIKNYD
GKVHGDRECGFINDEIFVELVNALGQYNDDDDDDDGDDPEEREEKQKDLED
HRDDKESRPPRKFPSDKIFEAISSMFPDKGTAEELKEKYKELTEQQLPGALPPE
CTPNTDGPNAKSVQREQSLHSFHTLFCRRCFKYDCFLHRKCNYSFHATPNTYK
RKNTETALDNKPCGPQCYQHLEGAKEFAAALTAERIKTPPKRPGGRRRGRLPN
NSSRPSTPTINVLESKDTDSDREAGTETGGENNDKEEEEKKDETSSSSEANSRC
QTPIKMKPNIEPPENVEWSGAEASMFRVLIGTYYDNFCAIARLIGTKTCRQVY
EFRVKESSIIAPAPAEDVDTPPRKKKRKHRLWAAHCRKIQLKKDGSSNHVYNY
QPCDHPRQPCDSSCPCVIAQNFCEKFCQCSSECQNRFPGCRCKAQCNTKQCPC
YLAVRECDPDLCLTCGAADHWDSKNVSCKNCSIQRGSKKHLLLAPSDVAGW
GIFIKDPVQKNEFISEYCGETISQDEADRRGKVYDKYMCSFLFNLNNDFVVDA
TRKGNKIRFANHSVNPNCYAKVMMVNGDHRIGIFAKRAIQTGEELFFDYRYS
QADALKYVGIEREMEIP
Human EZH2 coding sequence
(SEQ ID NO: 30)
atgggccagactgggaagaaatctgagaagggaccagtttgttggcggaagcgtgtaaaatcagagtacatgcga
ctgagacagctcaagaggttcagacgagctgatgaagtaaagagtatgtttagttccaatcgtcagaaaattttggaaagaa
cggaaatcttaaaccaagaatggaaacagcgaaggatacagcctgtgcacatcctgacttctgtgagctcattgcgcggga
ctagggagtgttcggtgaccagtgacttggattttccaacacaagtcatcccattaaagactctgaatgcagttgcttcagtac
ccataatgtattcttggtctcccctacagcagaattttatggtggaagatgaaactgttttacataacattccttatatgggagatg
aagttttagatcaggatggtactttcattgaagaactaataaaaaattatgatgggaaagtacacggggatagagaatgtggg
tttataaatgatgaaatttttgtggagttggtgaatgcccttggtcaatataatgatgatgacgatgatgatgatggagacgatc
ctgaagaaagagaagaaaagcagaaagatctggaggatcaccgagatgataaagaaagccgcccacctcggaaatttcc
ttctgataaaatttttgaagccatttcctcaatgtttccagataagggcacagcagaagaactaaaggaaaaatataaagaact
caccgaacagcagctcccaggcgcacttcctcctgaatgtacccccaacatagatggaccaaatgctaaatctgttcagag
agagcaaagcttacactcctttcatacgcttttctgtaggcgatgttttaaatatgactgcttcctacatcgtaagtgcaattattct
tttcatgcaacacccaacacttataagcggaagaacacagaaacagctctagacaacaaaccttgtggaccacagtgttac
cagcatttggagggagcaaaggagtttgctgctgctctcaccgctgagcggataaagaccccaccaaaacgtccaggag
gccgcagaagaggacggcttcccaataacagtagcaggcccagcacccccaccattaatgtgctggaatcaaaggatac
agacagtgatagggaagcagggactgaaacggggggagagaacaatgataaagaagaagaagagaagaaagatgaa
acttcgagctcctctgaagcaaattctcggtgtcaaacaccaataaagatgaagccaaatattgaacctcctgagaatgtgga
gtggagtggtgctgaagcctcaatgtttagagtcctcattggcacttactatgacaatttctgtgccattgctaggttaattggg
accaaaacatgtagacaggtgtatgagtttagagtcaaagaatctagcatcatagctccagctcccgctgaggatgtggata
ctcctccaaggaaaaagaagaggaaacaccggttgtgggctgcacactgcagaaagatacagctgaaaaaggacggct
cctctaaccatgtttacaactatcaaccctgtgatcatccacggcagccttgtgacagttcgtgcccttgtgtgatagcacaaa
atttttgtgaaaagttttgtcaatgtagttcagagtgtcaaaaccgctttccgggatgccgctgcaaagcacagtgcaacacca
agcagtgcccgtgctacctggctgtccgagagtgtgaccctgacctctgtcttacttgtggagccgctgaccattgggacag
taaaaatgtgtcctgcaagaactgcagtattcagcggggctccaaaaagcatctattgctggcaccatctgacgtggcaggc
tgggggatttttatcaaagatcctgtgcagaaaaatgaattcatctcagaatactgtggagagattatttctcaagatgaagctg
acagaagagggaaagtgtatgataaatacatgtgcagctttctgttcaacttgaacaatgattttgtggtggatgcaacccgc
aagggtaacaaaattcgttttgcaaatcattcggtaaatccaaactgctatgcaaaagttatgatggttaacggtgatcacagg
ataggtatttttgccaagagagccatccagactggcgaagagctgttttttgattacagatacagccaggctgatgccctgaa
gtatgtcggcatcgaaagagaaatggaaatcccttga
human CDYL protein sequence
(SEQ ID NO: 31)
MASEELYEVERIVDKRKNKKGKTEYLVRWKGYDSEDDTWEPEQHLVNC
EEYIHDFNRRHTEKQKESTLTRTNRTSPNNARKQISRSTNSNFSKTSPKALVIG
KDHESKNSQLFAASQKFRKNTAPSLSSRKNMDLAKSGIKILYPKSPYKSRTAVD
GFQSESPEKLDPVEQGQEDTVAPEVAAEKPVGALLGPGAERARMGSRPRIHPL
VPQVPGPVTAAMATGLAVNGKGTSPFMDALTANGTTNIQTSVTGVTASKRKFI
DDRRDQPFDKRLRFSVRQTESAYRYRDNVRKQDGFTHILLSTKSSENNSLNP
EVMREVQSALSTAAADDSKLVLLSAVGSYFCCGLDFIYFIRRLTDDRKRESTK
MAEAIRNFVNTFIQFKKPIIVAVNGPAIGLGASILPLCDVVWANEKAWFQTPYT
TFGQSPDGCSTVMFPKIMGGASANEMLLSGRKLTAQEACGKGLVSQVFWPGT
FTQEVMVRIKELASCNPVVLEESKALVRCNMKMELEQANERECEVLKKIWGS
AQGMDSMLKYLQRKIDEF
Human CDYL coding sequence
(SEQ ID NO: 32)
atggcttccgaggagctgtacgaggttgaaaggattgttgacaaaaggaaaaataaaaaagggaagacagagtatt
tggttcggtggaaaggctatgacagcgaggacgacacttgggagccggaacagcacctcgtgaactgtgaggaatacat
ccacgacttcaacagacgccacacggagaagcagaaggagagcacattgaccagaacaaacaggacctctcccaacaa
tgctaggaaacaaatctccagatccaccaacagcaacttttctaagacctctcctaaggcactcgtgattgggaaagaccac
gaatccaaaaacagccagctgtttgctgccagccagaagttcaggaagaacacagctccatctctctccagccggaagaa
catggacctagcgaagtcaggtatcaagatcctcgtgcctaaaagccccgttaagagcaggaccgcagtggacggctttc
agagcgagagccctgagaaactggaccccgtcgagcagggtcaggaggacacagtggcacccgaagtggcagcgga
aaagccggtcggagctttattgggccccggtgccgagagggccaggatggggagcaggcccaggatacacccactagt
gcctcaggtgcccggccctgtgactgcagccatggccacaggcttagctgttaacgggaaaggtacatctccgttcatgga
tgcattaacagccaatgggacaaccaacatacagacatctgttacaggagtgactgccagcaaaaggaaatttattgacga
cagaagagaccagccttttgacaagcgattgcgtttcagcgtgaggcaaacagaaagtgcctacagatacagagatattgt
ggtcaggaagcaggatggcttcacccacatcttgttatccacaaagtcctcagagaataactcactaaatccagaggtaatg
agagaagtccagagtgctctgagcacggccgctgccgatgacagcaagctggtactgctcagcgccgttggcagcgtctt
ctgttgtggacttgactttatttattttatacgacgtctgacagatgacaggaaaagagaaagcactaaaatggcagaagctat
cagaaacttcgtgaatactttcattcaatttaagaagcccattattgtagcagtcaatggcccagccattggtctaggagcatct
atattgcctctttgcgatgtggtttgggctaatgaaaaggcttggtttcaaacaccctataccaccttcggacagagtccagat
ggctgttctaccgttatgtttcccaagataatgggaggagcatctgcaaacgagatgctgctcagtggacggaagctgaca
gcgcaggaggcgtgtggcaagggcctggtctcccaggtgttttggcccgggacgttcactcaggaagtgatggttcgcatt
aaggagcttgcctcgtgcaatccagttgtgcttgaggaatccaaagccctcgtgcgctgcaacatgaagatggagctggag
caggccaacgagagggagtgtgaggtgctgaagaaaatctggggctcggcccaggggatggactccatgttaaagtact
tgcagaggaagatcgatgagttctga
human HMG20B protein sequence
(SEQ ID NO: 33)
MSHGPKQPGAAAAPAGGKAPGQHGGFVVTVKQERGEGPRAGEKGSHE
EEPVKKRGWPKGKKRKKILPNGPKAPVTGYVRFLNERREQIRTRHPDLPFPET
TKMLGAEWSKLQPTEKQRYLDEAEREKQQYMKELRAYQQSEAYKMCTEKIQ
EKKIKKEDSSSGLMNTLLNGHKGGDCDGFSTFDVPIFTEEFLDQNKAREAELR
RLRKMNVAFEEQNAVLQRHTQSMSSARERLEQELALEERRTLALQQQLQAVR
QALTASFASLPVPGTGETPTLGTLDFYMARLHGAIERDPAQHEKLNRIKEILA
QVASEHL
Human HMG20B coding sequence
(SEQ ID NO: 34)
atgtcccacggccccaagcagcccggcgcggccgccgcgccggcgggcggcaaggctccgggccagcatgg
gggcttcgtggtgactgtcaagcaagagcgcggcgagggtccacgcgcgggcgagaaggggtcccacgaggaggag
ccggtgaagaaacgcggctggcccaagggcaagaagcggaagaagattctgccgaatgggcccaaggcaccggtcac
gggctacgtgcgcttcctgaacgagcggcgcgagcagatccgcacgcgccacccggatctgccctttcccgagatcacc
aagatgctgggcgccgagtggagcaagctgcagccaacggaaaagcagcggtacctggatgaggccgagagagaga
agcagcagtacatgaaggagctgcgggcgtaccagcagtctgaagcctataagatgtgcacggagaagatccaggaga
agaagatcaagaaagaagactcgagctctgggctcatgaacactctcctgaatggacacaagggtggggactgcgatgg
cttctccaccttcgatgttcccatcttcactgaagagttcttggaccaaaacaaagcgcgtgaggcggagcttcggcgcttgc
ggaagatgaatgtggccttcgaggagcagaacgcggtactgcagaggcacacgcagagcatgagcagcgcgcgcgag
cgtctggagcaggagctggcgctggaggagcggaggacgctggcgctgcagcagcagctccaggccgtgcgccagg
cgctcaccgccagcttcgcctcactgccggtgccgggcacgggcgaaacgcccacgctgggcactctggacttctacat
ggcccggcttcacggagccatcgagcgcgaccccgcccagcacgagaagctcatcgtccgcatcaaggaaatcctggc
ccaggtcgccagcgagcacctgtga
human DPYSL2 protein sequence
(SEQ ID NO: 35)
MAERKQSGKAAEDEEVPAFFKNLGSGSPKPRQKFCGMFCPVEGSSENKT
IDFDSLSVGRGSGQVVAQQRDVAHLGPDPQPPYSRQGRRAGGEPSVESGRKV
EIRRASGKEALQNINDQSDRLLIKGGKIVNDDQSFYADIYMEDGLIKQIGENLI
VPGGVKTIEAHSRMVIPGGIDVHTRFQMPDQGMTSADDFFQGTKAALAGGTT
MIIDHVVPEPGTSLLAAFDQWREWADSKSCCDYSLHVDISEWHKGIQEEMEA
LVKDHGVNSFLVYMAFKDRFQLTDCQIYEVLSVIRDIGAIAQVHAENGDIIAEE
QQRILDLGITGPEGHVLSRPEEVEAEAVNRAITIANQTNCPLYITKVMSKSSAE
VIAQARKKGTVVYGEPITASLGTDGSHYWSKNWAKAAAFVTSPPLSPDPTTP
DFLNSLLSCGDLQVTGSAHCTFNTAQKAVGKDNFTLIPEGTNGTEERMSVIW
DKAVVTGKMDENQFVAVTSTNAAKVFNLYPRKGRIAVGSDADLVIWDPDSVK
TISAKTHNSSLEYNIFEGMECRGSPLVVISQGKIVLEDGTLHVTEGSGRYIPRKP
FPDFVYKRIKARSRLAELRGVPRGLYDGPVCEVSVTPKTVTPASSAKTSPAKQ
QAPPVRNLHQSGFSLSGAQIDDNIPRRTTQRIVAPPGGRANITSLG
Human DPYSL2 coding sequence
(SEQ ID NO: 36)
atggccgagagaaagcaatccgggaaggcggcagaggacgaagaggtccctgctttttttaaaaacctgggctcc
ggcagccccaagccccggcagaaattctgtggcatgttctgcccggtggaagggtcctcggagaacaagaccatcgactt
cgactcgctgtcggtgggccggggctcggggcaggtggtggctcagcagcgggacgtcgcccacttgggcccggacc
cgcagccgccgtactcgcggcagggccggcgcgccggcggagagccatctgttgaatcgggccggaaggtggagatc
cggagggcctcgggcaaagaagccctgcagaacatcaacgaccagagcgatcgtcttctgatcaaaggaggtaaaattg
ttaatgatgaccagtcgttctatgcagacatatacatggaagatgggttgatcaagcaaataggagaaaatctgattgtgcca
ggaggagtgaagaccatcgaggcccactcccggatggtgatccccggaggaattgacgtccacactcgtttccagatgcc
tgatcagggaatgacgtctgctgatgatttcttccaaggaaccaaggcggccctggctgggggaaccactatgatcattgac
cacgttgttcctgagcctgggacaagcctgctcgctgcctttgaccagtggagggaatgggccgacagcaagtcctgctgt
gactactctctgcatgtggacatcagtgagtggcataagggcatccaggaggagatggaagcgcttgtgaaggatcacgg
ggtaaattccttcctcgtgtacatggctttcaaagatcgcttccagctaacggattgccagatttatgaagtactgagtgtgatc
cgggatattggcgccatagcccaagtccacgcagaaaatggcgacatcattgcagaggagcagcagaggatcctggatc
tgggcatcacgggccccgagggacatgtgctgagccgacctgaggaggtcgaggccgaagccgtgaatcgtgccatca
ccatcgccaaccagaccaactgcccgctgtatatcaccaaggtgatgagcaaaagctctgctgaggtcatcgcccaggca
cggaagaagggaactgtggtgtatggcgagcccatcactgccagcttgggaacggacggctcccattactggagcaaga
actgggccaaggctgctgcctttgtcacctccccacccttgagccctgatccaaccactccagactttctcaactccttgctgt
cctgtggagacctccaggtcacgggcagtgcccattgcacgtttaacactgcccagaaggctgtaggaaaggacaacttc
accctgattccggagggcaccaatggcactgaggagcggatgtccgtcatctgggacaaggctgtggtcactgggaagat
ggatgagaaccagtttgtggctgtgaccagcaccaatgcagccaaagtcttcaacctttacccccggaaaggccgcattgc
tgtgggatccgatgccgacctggtcatctgggaccccgacagcgttaaaaccatctctgccaagacacacaacagctctct
cgagtacaacatctttgaaggcatggagtgccgcggctccccactggtggtcatcagccaggggaagattgtcctggagg
acggcaccctgcatgtcaccgaaggctctggacgctacattccccggaagcccttccctgattttgtttacaagcgtatcaag
gcaaggagcaggctggctgagctgagaggggttcctcgtggcctgtatgacggacctgtgtgtgaagtgtctgtgacgcc
caagacagtcactccagcctcctcggccaagacgtctcctgccaagcagcaggccccacctgtccggaacctgcaccag
tctggattcagtttgtctggtgctcagattgatgacaacattccccgccgcaccacccagcgtatcgtggcgccccccggtg
gccgtgccaacatcaccagcctgggctag
human BAF45b protein sequence
(SEQ ID NO: 37)
MATVIPGPLSLGEDFYREAIEHCRSYNARLCAERSLRLPFLDSQTGVAQN
NCYIWMEKTHRGPGLAPGQIYTYPARCWRKKRRLNILEDPRLRPCEYKIDCE
APLKKEGGLPEGPVLEALLCAETGEKKIELKEEETIMDCQKQQLLEFPHDLEV
EDLEDDIPRRKNRAKGKAYGIGGLRKRQDTASLEDRDKPYYCDICGKRYKNR
PGLSYHYTHTHLAEEEGEENAERHALPFHRKNNHKQFYKELAWVPEAQRKH
TAKKAPDGTVIPNGYCDFCLGGSKKTGCPEDLISCADCGRSGHPSCLQFTVN
MTAAVRTYRWQCIECKSCSLCGTSENDDQLLFCDDCDRGYHMYCLSPPMAEP
PEGSWSCHLCLRHLKEKASAYITLT
Human BAF45b coding sequence
(SEQ ID NO: 38)
atggccactgtcatccctggccccctgagcctaggcgaggacttctaccgcgaggccatcgagcactgccgcagtt
acaacgcgcgcctgtgcgccgagcgcagcctgcgactgcccttcctcgactcgcagaccggcgtggcccagaacaact
gctacatctggatggagaagacccaccgcgggccgggtttggccccgggacagatttacacgtaccccgcccgctgttg
gaggaagaaacggagactcaacatcctggaggaccccagactcaggccctgcgagtacaagatcgactgtgaagcacc
cctgaagaaggagggtggcctcccggaagggccggtcctcgaggctctactgtgtgcagagacgggggagaagaagat
tgagctgaaggaggaggagaccattatggactgtcagaaacagcagttgctggagtttccgcatgacctcgaggtggaag
acttggaggatgacattcccaggaggaagaacagggccaaaggaaaggcatatggcatcgggggtctccggaaacgcc
aggacaccgcttccctggaggaccgagacaagccgtatgtctgtgatatctgtgggaaacggtataagaaccggccggg
gctcagctaccactacacccacacccacctggccgaggaggagggggaggagaacgccgaacgccacgccctgccct
tccaccggaaaaacaaccataaacagttttacaaagaattggcctgggtccctgaggcacaaaggaaacacacagccaag
aaggcgcccgacggcactgtcatccccaacggctactgtgacttctgcctggggggctccaagaagacggggtgtcccg
aggacctcatctcctgtgcggactgtgggcgatcaggacacccctcgtgtttacaattcacggtgaacatgacggcagccg
tgcggacctaccgctggcagtgcatcgagtgcaaatcctgcagcctgtgcggaacctccgagaacgacgaccagctgct
gttttgtgatgactgcgatcggggttaccacatgtactgcctgagtccccccatggcggagcccccggaagggagctgga
gctgtcacctctgtctccggcacctgaaggaaaaggcttctgcttacatcaccctcacctag
human SCF protein sequence
(SEQ ID NO: 39)
MKKTQTWILTCIYLQLLLFNPLVKTEGICRNRVTNNVKDVTKLVANLPKD
YMITLKYVPGMDVLPSHCWISEMWQLSDSLTDLLDKFSNISEGLSNYSIIDKL
VNIVDDLVECVKENSSKDLKKSFKSPEPRLFTPEEFFRIFNRSIDAFKDFWASE
TSDCVVSSTLSPEKDSRVSVTKPFMLPPVAASSLRNDSSSSNRKAKNPPGDSSL
HWAAMALPALFSLIIGFAFGALYWKKRQPSLTRAVENIQINEEDNEISMLQEKE
REFQEV
Human SCF coding sequence
(SEQ ID NO: 40)
atgaagaagacacaaacttggattctcacttgcatttatcttcagctgctcctatttaatcctctcgtcaaaactgaaggg
atctgcaggaatcgtgtgactaataatgtaaaagacgtcactaaattggtggcaaatcttccaaaagactacatgataaccct
caaatatgtccccgggatggatgttttgccaagtcattgttggataagcgagatggtagtacaattgtcagacagcttgactga
tcttctggacaagttttcaaatatttctgaaggcttgagtaattattccatcatagacaaacttgtgaatatagtggatgaccttgt
ggagtgcgtgaaagaaaactcatctaaggatctaaaaaaatcattcaagagcccagaacccaggctctttactcctgaaga
attctttagaatttttaatagatccattgatgccttcaaggactttgtagtggcatctgaaactagtgattgtgtggtttcttcaacat
taagtcctgagaaagattccagagtcagtgtcacaaaaccatttatgttaccccctgttgcagccagctcccttaggaatgac
agcagtagcagtaataggaaggccaaaaatccccctggagactccagcctacactgggcagccatggcattgccagcatt
gttttctcttataattggctttgcttttggagccttatactggaagaagagacagccaagtcttacaagggcagttgaaaatatac
aaattaatgaagaggataatgagataagtatgttgcaagagaaagagagagagtttcaagaagtgtaa
human HuB protein sequence
(SEQ ID NO: 41)
METQLSNGPTCNNTANGPTTINNNCSSPVDSGNTEDSKTNLIVNYLPQN
MTQEELKSLFGSIGEIESCKLVRDKITGQSLGYGFVNYIDPKDAEKAINTLNGL
RLQTKTIKYSYARPSSASLRDANLYVSGLPKTMTQKELEQLFSQYGRIITSRILV
DQVTGISRGVGFIRFDKRIEAEEAIKGLNGQKPPGATEPITVKFANNPSQKTNQ
AILSQLYQSPNRRYPGPLAQQAQRFRLDNLLNMAYGYKRFSPMTIDGMTSLA
GINIPGHPGTGWCIFVYNLAPDADESILWQMFGPFGAVTNVKVIRDFNTNKCK
GFGFVTMTNYDEAAMAIASLNGYRLGDRVLQVSFKTNKTHKA
Human HuB coding sequence
(SEQ ID NO: 42)
atggaaacacaactgtctaatgggccaacttgcaataacacagccaatggtccaaccaccataaacaacaactgttc
gtcaccagttgactctgggaacacagaagacagcaagaccaacttaatagtcaactaccttcctcagaacatgacacagga
ggaactaaagagtctctttgggagcattggtgaaatagagtcctgtaagcttgtaagagacaaaataacagggcagagcttg
ggatatggctttgtgaactacattgaccccaaggatgcagagaaagctatcaacaccctgaatggattgagacttcaaacca
aaacaataaaagtttcctatgctcgcccaagttcagcttctatcagagatgcaaatttatatgtcagcggacttccaaaaacaat
gacccagaaggagttggaacagcttttttcacaatatggacgcattattacttctcgtattcttgtcgaccaggtcactggcata
tcaaggggtgtagggtttattcgatttgacaagcgaattgaggcagaagaagctatcaaaggcctaaatggccagaaacct
cccggtgccacggagccaatcactgtaaagtttgctaataacccaagccaaaaaaccaatcaggccatcctttcccagctgt
accagtctccaaacagaaggtatccaggaccgctagctcagcaggcacagcgttttaggttggacaatctgctcaatatgg
cttatggagtaaagaggttttctccaatgaccattgacggaatgaccagtttggctggaattaatatccctgggcaccctgga
acagggtggtgtatatttgtgtacaacctggctcctgacgcagatgagagtatcctgtggcaaatgtttgggccttttggagct
gtcaccaatgtgaaggtcatccgtgactttaacaccaataaatgcaaaggttttggatttgtgactatgacaaactatgatgag
gctgccatggcgatagctagcctcaatggataccgtctgggagacagagtactgcaggtctcctttaagacaaacaaaacg
cacaaagcctaa
human HuC protein sequence
(SEQ ID NO: 43)
MVTQILGAMESQVGGGPAGPALPNGPLLGTNGATDDSKTNLIYNYLPQN
MTQDEFKSLFGSIGDIESCKLYRDKITGQSLGYGFVNYSDPNDADKAINTLNG
LKLQTKTIKVSYARPSSASIRDANLYYSGLPKTMSQKEMEQLFSQYGRIITSRIL
VDQVTGVSRGVGFIRFDKRIEAEEAIKGLNGQKPLGAAEPITYKFANNPSQKT
GQALLTHLYQSSARRYAGPLHHQTQRFRLDNLLNMAYGVKSPLSLIARFSPIAI
DGMSGLAGYGLSGGAAGAGWCIFYYNLSPEADESVLWQLFGPFGAVTNYKV
IRDFTTNKCKGFGFVTMTNYDEAAMAIASLNGYRLGERVLQVSFKTSKQHKA
Human HuC coding sequence
(SEQ ID NO: 44)
atggtcactcagatactgggggccatggagtctcaggtgggggggggcccggccggcccggccctgcccaacg
ggccactccttggtacaaatggagccactgacgacagcaagaccaacctcatcgtcaactacctgccccagaacatgacc
caggatgagttcaagagtctcttcggcagcattggcgacatcgagtcctgcaagttggttcgggacaagatcacagggcag
agccttggctacgggtttgtgaactattctgaccccaatgatgcagacaaagccatcaacaccctcaacggcctcaaattac
agacgaagaccatcaaggtgtcctatgccagacccagttcagcatccatccgggatgctaacctgtacgtcagcgggctcc
ccaagaccatgagccagaaagagatggagcagctcttctcccagtacggccgcatcatcacgtcccgcatcctggtggac
caggtcacaggtgtctctcggggtgtgggattcatccgctttgacaagaggattgaggccgaagaggctatcaaaggactg
aatgggcagaagccgctgggcgcagctgagcccatcacagtcaagttcgcgaacaacccaagtcagaagacggggca
ggcgctgctcacccacctctaccagtcatccgcccggcgctacgcaggccccctacaccatcagacccagcgtttccggc
tggacaatttgctcaacatggcctacggcgtcaagagtcccctgtcgctcatcgccaggttctcgccgatcgccatcgatgg
tatgagcggcctggcgggcgtgggcctgtcggggggcgcggcgggcgccggctggtgcatcttcgtgtacaacctgtca
ccggaggcagacgagagcgtgctgtggcagctgttcgggccttttggggcagtcaccaacgtcaaggtcatccgtgatttc
accaccaacaagtgcaagggtttcggcttcgtgaccatgaccaactatgacgaggcggccatggccatcgccagcctgaa
cggctatcgcctgggcgagcgcgtgctgcaggtctccttcaagaccagcaaacagcacaaggcgtga
human HuD protein sequenc
(SEQ ID NO: 45)
MVMIISTMEPQVSNGPTSNTSNGPSSNNRNCPSPMQTGATTDDSKTNLIV
NYLPQNMTQEEFRSLFGSIGEIESCKLVRDKITGQSLGYGFVNYIDPKDAEKAI
NTLNGLRLQTKTIKVSYARPSSASIRDANLYYSGLPKTMTQKELEQLFSQYGRI
ITSRILVDQVTGYSRGYGFIRFDKRIEAEEAIKGLNGQKPSGATEPITVKFANNP
SQKSSQALLSQLYQSPNRRYPGPLHHQAQRFRLDNLLNMAYGVKRLMSGPVP
PSACPPRFSPITIDGMTSLYGMNIPGHTGTGWCIFVYNLSPDSDESVLWQLFGP
FGAVNNVKVIRDFNTNKCKGFGFVTMTNYDEAAMAIASLNGYRLGDRVLQV
SFKTNKAHKS
Human HuD coding sequence
(SEQ ID NO: 46)
atggttatgataattagcaccatggagcctcaggtgtcaaatggtccgacatccaatacaagcaatggaccctccag
caacaacagaaactgtccttctcccatgcaaacaggggcaaccacagatgacagcaaaaccaacctcatcgtcaactattt
accccagaatatgacccaagaagaattcaggagtctcttcgggagcattggtgaaatagaatcctgcaaacttgtgagaga
caaaattacaggacagagtttagggtatggatttgttaactatattgatccaaaggatgcagagaaagccatcaacactttaaa
tggactcagactccagaccaaaaccataaaggtctcatatgcccgtccgagctctgcctcaatcagggatgctaacctctat
gttagcggccttcccaaaaccatgacccagaaggaactggagcaacttttctcgcaatacggccgtatcatcacctcacga
atcctggttgatcaagtcacaggagtgtccagaggggtgggattcatccgctttgataagaggattgaggcagaagaagcc
atcaaagggctgaatggccagaagcccagcggtgctacggaaccgattactgtgaagtttgccaacaaccccagccaga
agtccagccaggccctgctctcccagctctaccagtcccccaaccggcgctacccaggtccacttcaccaccaggctcag
aggttcaggctggacaatttgcttaatatggcctatggcgtaaagagactgatgtctggaccagtccccccttctgcttgtccc
cccaggttctccccaattaccattgatggaatgacaagccttgtgggaatgaacatccctggtcacacaggaactgggtggt
gcatctttgtctacaacctgtcccccgattccgatgagagtgtcctctggcagctctttggcccctttggagcagtgaacaacg
taaaggtgattcgtgacttcaacaccaacaagtgcaagggattcggctttgtcaccatgaccaactatgatgaggcggccat
ggccatcgccagcctcaacgggtaccgcctgggagacagagtgttgcaagtttcctttaaaaccaacaaagcccacaagt
cctga
human CYP1B1 protein sequence
(SEQ ID NO: 47)
MGTSLSPNDPWPLNPLSIQQTTLLLLLSVLATVHVGQRLLRQRRRQLRSA
PPGPFAWPLIGNAAAVGQAAHLSFARLARRYGDVFQIRLGSCPIVVLNGERAIH
QALVQQGSAFADRPAFASFRVYSGGRSMAFGHYSEHWKVQRRAAHSMMRNF
FTRQPRSRQVLEGHVLSEARELVALLVRGSADGAFLDPRPLTVVAVANVMSAV
CFGCRYSHDDPEFRELLSHNEEFGRTVGAGSLVDVMPWLQYFPNPVRTVFRE
FEQLNRNFSNFILDKFLRHCESLRPGAAPRDMMDAFILSAEKKAAGDSHGGG
ARLDLENVPATITDIFGASQDTLSTALQWLLLLFTRYPDVQTRVQAELDQVYG
RDRLPCMGDQPNLPYVLAFLYEAMRFSSFVPVTIPHATTANTSVLGYHIPKDT
VVFVNQWSVNHDPLKWPNPENFDPARFLDKDGLINKDLTSRVMIFSVGKRRC
IGEELSKMQLFLFISILAHQCDFRANPNEPAKMNFSYGLTIKPKSFKVNVTLRE
SMELLDSAVQNLQAKETCQ
Human CYP1B1 coding sequence
(SEQ ID NO: 48)
atgggcaccagcctcagcccgaacgacccttggccgctaaacccgctgtccatccagcagaccacgctcctgcta
ctcctgtcggtgctggccactgtgcatgtgggccagcggctgctgaggcaacggaggcggcagctccggtccgcgcccc
cgggcccgtttgcgtggccactgatcggaaacgcggcggcggtgggccaggcggctcacctctcgttcgctcgcctggc
gcggcgctacggcgacgttttccagatccgcctgggcagctgccccatagtggtgctgaatggcgagcgcgccatccac
caggccctggtgcagcagggctcggccttcgccgaccggccggccttcgcctccttccgtgtggtgtccggcggccgca
gcatggctttcggccactactcggagcactggaaggtgcagcggcgcgcagcccacagcatgatgcgcaacttcttcacg
cgccagccgcgcagccgccaagtcctcgagggccacgtgctgagcgaggcgcgcgagctggtggcgctgctggtgcg
cggcagcgcggacggcgccttcctcgacccgaggccgctgaccgtcgtggccgtggccaacgtcatgagtgccgtgtgt
ttcggctgccgctacagccacgacgaccccgagttccgtgagctgctcagccacaacgaagagttcgggcgcacggtgg
gcgcgggcagcctggtggacgtgatgccctggctgcagtacttccccaacccggtgcgcaccgttttccgcgaattcgag
cagctcaaccgcaacttcagcaacttcatcctggacaagttcttgaggcactgcgaaagccttcggcccggggccgcccc
ccgcgacatgatggacgcctttatcctctctgcggaaaagaaggcggccggggactcgcacggtggtggcgcgcggctg
gatttggagaacgtaccggccactatcactgacatcttcggcgccagccaggacaccctgtccaccgcgctgcagtggct
gctcctcctcttcaccaggtatcctgatgtgcagactcgagtgcaggcagaattggatcaggtcgtggggagggaccgtct
gccttgtatgggtgaccagcccaacctgccctatgtcctggccttcctttatgaagccatgcgcttctccagctttgtgcctgtc
actattcctcatgccaccactgccaacacctctgtcttgggctaccacattcccaaggacactgtggtttttgtcaaccagtggt
ctgtgaatcatgacccactgaagtggcctaacccggagaactttgatccagctcgattcttggacaaggatggcctcatcaa
caaggacctgaccagcagagtgatgattttttcagtgggcaaaaggcggtgcattggcgaagaactttctaagatgcagctt
tttctcttcatctccatcctggctcaccagtgcgatttcagggccaacccaaatgagcctgcgaaaatgaatttcagttatggtc
taaccattaaacccaagtcatttaaagtcaatgtcactctcagagagtccatggagctccttgatagtgctgtccaaaatttaca
agccaaggaaacttgccaataa
human BTRC protein sequence
(SEQ ID NO: 49)
MDPAEAVLQEKALKFMCSMPRSLWLGCSSLADSMPSLRCLYNPGTGALT
AFQNSSEREDCNNGEPPRKIIPEKNSLRQTYNSCARLCLNQETVCLASTAMKT
ENCYAKTKLANGTSSMIYPKQRKLSASYEKEKELCYKYFEQWSESDQVEFVE
HLISQMCHYQHGHINSYLKPMLQRDFITALPARGLDHIAENILSYLDAKSLCA
AELVCKEWYRVTSDGMLWKKLIERMVRTDSLWRGLAERRGWGQYLFKNKP
PDGNAPPNSFYRALYPKIIQDIETIESNWRCGRHSLQRIHCRSETSKGVYCLQY
DDQKIVSGLRDNTIKIWDKNTLECKRILTGHTGSVLCLQYDERVIITGSSDSTV
RVWDVNTGEMLNTLIHHCEAVLHLRFNNGMMVTCSKDRSIAVWDMASPTDI
TLRRVLVGHRAAVNVVDFDDKYTVSASGDRTIKVWNTSTCEFVRTLNGHKRG
IACLQYRDRLVVSGSSDNTIRLWDIECGACLRVLEGHEELVRCIRFDNKRIVSG
AYDGKIKVWDLVAALDPRAPAGTLCLRTLVEHSGRVFRLQFDEFQTYSSSHDD
TILIWDFLNDPAAQAEPPRSPSRTYTYISR
Human BTRC coding sequence
(SEQ ID NO: 50)
atggacccggccgaggcggtgctgcaagagaaggcactcaagtttatgtgctctatgcccaggtctctgtggctgg
gctgctccagcctggcggacagcatgccttcgctgcgatgcctgtataacccagggactggcgcactcacagctttccaga
attcctcagagagagaagactgtaataatggcgaaccccctaggaagataataccagagaagaattcacttagacagacat
acaacagctgtgccagactctgcttaaaccaagaaacagtatgtttagcaagcactgctatgaagactgagaattgtgtggc
caaaacaaaacttgccaatggcacttccagtatgattgtgcccaagcaacggaaactctcagcaagctatgaaaaggaaaa
ggaactgtgtgtcaaatactttgagcagtggtcagagtcagatcaagtggaatttgtggaacatcttatatcccaaatgtgtcat
taccaacatgggcacataaactcgtatcttaaacctatgttgcagagagatttcataactgctctgccagctcggggattggat
catattgctgagaacattctgtcatacctggatgccaaatcactatgtgctgctgaacttgtgtgcaaggaatggtaccgagtg
acctctgatggcatgctgtggaagaagcttatcgagagaatggtcaggacagattctctgtggagaggcctggcagaacg
aagaggatggggacagtatttattcaaaaacaaacctcctgacgggaatgctcctcccaactctttttatagagcactttatcct
aaaattatacaagacattgagacaatagaatctaattggagatgtggaagacatagtttacagagaattcactgccgaagtga
aacaagcaaaggagtttactgtttacagtatgatgatcagaaaatagtaagcggccttcgagacaacacaatcaagatctgg
gataaaaacacattggaatgcaagcgaattctcacaggccatacaggttcagtcctctgtctccagtatgatgagagagtgat
cataacaggatcatcggattccacggtcagagtgtgggatgtaaatacaggtgaaatgctaaacacgttgattcaccattgtg
aagcagttctgcacttgcgtttcaataatggcatgatggtgacctgctccaaagatcgttccattgctgtatgggatatggcctc
cccaactgacattaccctccggagggtgctggtcggacaccgagctgctgtcaatgttgtagactttgatgacaagtacattg
tttctgcatctggggatagaactataaaggtatggaacacaagtacttgtgaatttgtaaggaccttaaatggacacaaacga
ggcattgcctgtttgcagtacagggacaggctggtagtgagtggctcatctgacaacactatcagattatgggacatagaat
gtggtgcatgtttacgagtgrtagaaggccatgaggaattggtgcgttgtattcgatttgataacaagaggatagtcagtggg
gcctatgatggaaaaattaaagtgtgggatcttgtggctgctttggacccccgtgctcctgcagggacactctgtctacggac
ccttgtggagcattccggaagagtttttcgactacagtttgatgaattccagattgtcagtagttcacatgatgacacaatcctc
atctgggacttcctaaatgatccagctgcccaagctgaacccccccgttccccttctcgaacatacacctacatctccagata
a

1. A method of transdifferentiating mammalian non-neuronal cells into neurons, which comprises:
provide a negative regulator that can reduce the expression of negative regulatory gene, wherein the negative regulatory gene includes RCOR1, RCOR2, RCOR3, Sin3a, Sin3b, HDAC1, HDAC2, KDM1A, PHF21A, BAF53a, G9a, USP14, HuR, BrG1, EZH42, CDYL, or HMG20B, and/or
provide a positive regulator that can increase the expression of positive regulatory gene, wherein the positive regulatory gene includes DPYSL2, BAF45b, SCF, HuB, HuC, HuD, CYP1B1, or BTRC,
an effective amount of the negative regulator or the positive regulator is brought to contact with the non-neuronal cells to induce trans-differentiation of the non-neuronal animal cells into neurons;
preferably, the non-neuronal cells are derived from human.

2. (canceled)

3. The method of claim 1, wherein, the non-neuronal cells are stem cells, progenitor cells, or terminally differentiated cells; preferably, are glial cells; more preferably, the glial cells are astrocytes, oligodendrocytes, microglia, NG2 cells, Müller glial cells, glioma cells, or spiral ganglion glial cells; even more preferably, the glial cells are astrocytes or Müller glial cells, or
the non-neuronal cells are from brain, preferably, the non-neuronal cells are from cerebrum, midbrain, cerebellum, brainstem, or spinal cord; more preferably, from the striatum or substantia nigra in brain; or
the non-neuronal cells are astrocytes from the brain, preferably astrocytes are from the striatum or the substantia nigra; or
the non-neuronal cells are from the eye, preferably, the non-neuronal cells are Müller glial cells from the eye.

4-6. (canceled)

7. The method according to claim 1, wherein, the neurons are dopaminergic neurons, retinal ganglion cells, photoreceptor cells, 5-HT neurons, NE neurons, ChAT neurons, motor neurons, GABA neurons, glutamatergic neurons, spinal cord neurons, spinal motor neurons, spinal sensory neurons, bipolar cells, amacrine cells, cochlear nerve cells, pyramidal neurons, interneurons, medium spiny neurons, Purkinje cells, granule cells, olfactory receptor neurons, or periglomerular cells, or combinations thereof.

8. The method according to claim 1, wherein, the negative regulatory gene is RCOR1, RCOR2, RCOR3, Sin3a, Sin3b, KDM1A, BAF53a, G9a, HuR, BrG1, or EZH2, and the non-neuronal animal cells are from brain or eyes.

9. The method of claim 8, wherein the negative regulatory gene is RCOR1, RCOR2, RCOR3 or G9a, and the non-neuronal animal cells are from the brain; preferably, the non-neuronal cells are astrocytes from brain; more preferably, the non-neuronal cells are astrocytes from the striatum or substantia nigra; or
wherein, the negative regulatory gene is Sin3a, Sin3b, KDM1A, BAF53a, G9a, HuR, BrG1, CDYL, or EZH2, and the non-neuronal cells are from eyes; preferably, the non-neuronal cells are Müller glial cells from eyes; or
wherein the negative regulatory gene is HuR, the neuron is retinal ganglion cell; or
wherein the negative regulatory gene is Sin3a, KDM1A, BAF53a, G9a, HuR, BrG1, CDYL, or EZH2, the neuron is photoreceptor cell.

10-12. (canceled)

13. The method according to claim 1, wherein, the positive regulatory gene is DPYSL2, BAF45b, SCF, HuB, HuC, HuD, CYP1B1, or BTRC, and the non-neuronal cells are from brain; preferably, the non-neuronal cells are from the striatum, substantia nigra, ventral tegmental area of the midbrain, spinal cord, hypothalamus, dorsal midbrain, cerebral cortex, hippocampus, or cerebellum; more preferably, from the striatum.

14. The method of claim 13, wherein, the positive regulatory gene is SCF or HuB; or
the positive regulatory gene is HuB or BTRC, and an effective amount of the positive regulator contacts with the non-neuronal cell to induce non-neuronal cell transdifferentiating into dopaminergic neurons.

15. (canceled)

16. The method according to claim 13, wherein, the non-neuronal cells are astrocytes; more preferably, the non-neuronal cells are astrocytes from the striatum or the substantia nigra.

17. The method according to claim 1, wherein, the positive regulatory gene is DPYSL2, BAF45b, SCF, HuC, HuD, or CYP1B1, and the non-neuronal cells are from eyes; preferably, the non-neuronal cells are Müller glial cells from eyes.

18. The method of claim 17, wherein, the positive regulatory gene is SCF, HuD, or CYP1B1, and the neuron is retinal ganglion cell; or
the positive regulatory gene is DPYSL2 or BAF45b, and the neuron is photoreceptor cell.

19. (canceled)

20. (canceled)

21. The method of claim 1, wherein, the negative regulator selects from gene editing tools or epigenetic regulatory tools for reducing the expression of negative regulatory gene; inhibitors of negative regulatory gene, activity inhibitors of negative regulator gene, or degradation activators of the protein encoded by a negative regulatory gene;
preferably, the inhibitor is: an inhibitory antibody of the negative regulatory gene; or a small molecule inhibitor of the negative regulatory gene; or an inhibitory mRNA, microRNA, siRNA, shRNA, antisense oligonucleotide, binding protein or protein domain, polypeptide, nucleic acid aptamer, or PROTAC of the negative regulatory gene; or inhibitory binding protein or ligand of the negative regulatory gene.

22. (canceled)

23. The method of claim 21, wherein, the negative regulator contains the gRNA as shown in any one of SEQ ID NO: 51-67 and gene editing protein.

24. The method of claim 1, wherein, the positive regulator selects from epigenetic regulatory tools capable of increasing expression of positive regulatory gene, expression activators of positive regulatory gene, degradation inhibitors of proteins encoded by positive regulatory gene, mRNA stabilizers of positive regulatory gene, or exogenous positive regulatory gene or functional fragment of positive regulatory gene,
preferably the activator is: an agonistic antibody of positive regulatory gene; or a small molecule agonist of positive regulatory gene; or an agonistic binding protein or ligand of positive regulatory gene; or an inhibitor of competitive genes of positive regulatory gene.

25. (canceled)

26. The method of claim 1, wherein, the positive regulator contains a nucleic acid sequence shown in SEQ ID NO: 36, 38, 40, 42, 44, 46, 48, or 50, or contains a functional fragment of the nucleic acid sequence shown in SEQ ID NO: 36, 38, 40, 42, 44, 46, 48, or 50.

27. The method of claim 21, wherein, the gene editing tool includes a gene editing system or its expression vector, the gene editing system selects from: CRISPR system (including CRISPR/Cas system), ZFN system, TALEN system, or combination thereof,
preferably a CRISPR system is used to reduce the expression or activity of negative regulatory gene; preferably, the CRISPR system contains a nucleic acid encoding a Cas enzyme or a nucleic acid encoding the functional domain of Cas enzyme, as well as gRNA targeting the cellular transdifferentiation factor; more preferably, the Cas enzyme is Cas13; more preferably, the Cas enzyme is Cas13d, Cas13X, Cas13a, Cas13b, Cas13c, or Cas13Y; even more preferably, the Cas enzyme is CasRx.

28. (canceled)

29. The method of claim 1, wherein, the negative regulator or positive regulator is carried by a carrier; preferably, the carrier is viral vector, lipid nanoparticle (LNP), lipidosome, cationic polymer (such as PEI), nanoparticle, exosome, or virus-like particle: more preferably, the carrier is AAV vector or lipid nanoparticle.

30. The method of claim 1, wherein the effective amount of the negative regulator or the positive regulator contacts with the non-neuronal cells in vitro, to induce non-neuronal cells transdifferentiating into neurons in vitro; or
the effective amount of the negative regulator or the positive regulator contacts the non-neuronal cells in vivo, to induce the non-neuronal cells transdifferentiating into neurons in vivo.

31-36. (canceled)

37. A method for preventing or treating diseases related to neuronal injury or neuronal death, comprising administering an effective amount of the negative regulator or positive regulator of claim 1 to a subject in need;
wherein, preferably the disease related to neuronal injury or neuronal death selects from Parkinson's disease, visual system diseases related to function loss or death of RGC or photoreceptor cell, Alzheimer's disease, brain injury, Huntington's disease, epilepsy, depression, sleep disorders, cerebral ischemia, motor neuron disease, amyotrophic lateral sclerosis, spinal muscular atrophy, ataxia, PloyQ disease, schizophrenia, addiction, Pick's disease, blindness, deafness; preferably selected from Parkinson's disease and visual system diseases related to function loss or death of RGC or photoreceptor cell;
more preferably the visual system disease related to function loss or death of RGC preferably selects from: vision impairment due to the death of RGC cell, glaucoma, age-related RGC pathology, optic nerve injury, age-related macular degeneration (AMD), diabetes-related retinopathy, retinal ischemia or hemorrhage, Leber's hereditary optic neuropathy, or combinations thereof; the visual system disease related to the function loss or death of photoreceptor cell preferably selects from: photoreceptor cell degeneration or death caused by injury or degenerative disease, macular degeneration, retinitis pigmentosa, diabetes-related blindness, nyctalopia, color blindness, hereditary blindness, congenital amaurosis, or combinations thereof.

38. (canceled)

39. (canceled)

40. The method of claim 37, wherein, the neurons are dopaminergic neurons, 5-HT neurons, NE neurons, ChAT neurons, GABA neurons, glutamatergic neurons, motor neurons, photoreceptor cells (such as rods and cones), retinal ganglion cells (RGC), cochlear nerve cells (such as cochlear spiral ganglion cells and vestibular neurons), or medium spiny neurons (MSN) or combinations thereof, preferably, are dopaminergic neurons, retinal ganglion cells or photoreceptor cells.
